(19) United States
(12) Patent Application Publication
(10) Pub. No.: US 2008/0305493 A1
(43) Pub. Date: Dec. 11, 2008

(54) Determining Cancer-Linked Genes and Therapeutic Targets Using Molecular Cytogenetic Methods

(75) Inventors: Jeffrey W. Strovel, Laurel, MD (US); Colyn B. Cain, Bethesda, MD (US); Stephen K. Horrigan, Poolesville, MD (US); Meena Augustus, Gaithersburg, MD (US)

Correspondence Address: CARELLA, BYRNE, BAIN, GILFILLAN, CECCHI,;STEWART & OLSTEIN, 5 BECKER FARM ROAD, ROSELAND, NJ, 07068, US

(73) Assignee: AVALON PHARMACEUTICALS, Germantown, MD (US)

(21) Appl. No.: 10/591,049

(22) PCT Filed: Mar. 8, 2005
(86) PCT No.: PCT/US2005/007748

§ 371 Date: May 5, 2008

Related U.S. Application Data
(60) Provisional application No. 60/550,304, filed on Mar. 8, 2004.

(51) Int. Cl. G01N 33/53; C12Q 1/68; C12Q 1/02; C12N 5/06; G01N 33/566

(52) U.S. Cl. 435/7.1; 435/6; 436/501; 435/29; 435/375

(57) ABSTRACT

Methods for identifying antineoplastic agents by using their ability to modify expression of specific genes or the biological activity of polypeptides encoded by such genes, wherein said genes are located in specific chromosomal regions, called amplicons, or regions of interest, and the presence of such amplified regions within a cancerous cell, are disclosed. Also described are methods for diagnosing cancerous, or potentially cancerous, conditions using these methods. Also encompassed are methods involving determining the modulated expression of the genes in these regions of interest (ROIs), or amplicons, as pharmacodynamic/pharmacogenetic/surrogate markers and/or for patient profiling prior to accrual for clinical trials/treatments based on the identification of these genes as validated gene/drug targets in various cancer tissue types.

This application claims priority of U.S. Provisional Application Ser. No. 60/550,304, filed 8 Mar. 2004, the disclosure of which is hereby incorporated by reference in its entirety.

### FIELD OF THE INVENTION

The present invention relates to Identification of amplifications/gains of genomic segments of DNA within human chromosomes in diseased states, such as cancer, that are demarcated and limited within specific chromosomal bands and defined herein as “amplicons” and whose disruption and/or change in expression is useful to distinguish cancerous from non-cancerous tissue and serve as potential therapeutic targets, pharmacodynamic/pharmacogenetic/surrogate and prognostic and diagnostic markers.

### BACKGROUND OF THE INVENTION

Malignant tumors are a leading cause of death in the United States and one in four Americans is likely to die of cancer. This disease is often characterized by an increase in the number of abnormal, neoplastic cells that are ultimately derived from a normal tissue after which the cells proliferate to form a tumor, which can then metastasize (spreading into adjacent tissues or traveling elsewhere in the body via the bloodstream or lymphatic system).

The genomes of various well-studied tumors carry several different independently altered genes, including activated oncogenes and inactivated tumor suppressor genes. Chromosomal abnormalities have been identified in most cancer cells. Conventional chromosome banding techniques allow for the detection of specific chromosomal defects in tumor cells but interpretation of the banding pattern is sometimes difficult, particularly when complex chromosomal rearrangements or subtle abnormalities are present. In recent years, new techniques, such as CGH and SKY, based on fluorescent in situ hybridization (FISH) (Pinkel et al., Proc Nat Acad Sci USA 85:9138-42 (1988)) have been developed to overcome the limitations of conventional chromosome banding. CGH measures intensities of fluorescently labeled tumor DNA and normal DNA following hybridization to normal chromosomes (Kallioniemi et al., Science 258:818-21 (1992)). Gain or loss of copy number of a particular chromosome or chromosome region in the tumor DNA is determined by the relative intensity of a fluorescence ratio. SKY utilizes a cocktail of chromosome probes, fluorescently labeled to specify each chromosome, which is hybridized to tumor chromosomes in an effort to identify numerical and structural abnormalities in the tumor cell (Schröck et al., Science 273:494-7 (1996)). CGH and SKY have been used to identify chromosomal regions that harbor genes significant to the process of tumor initiation or progression.

The identification of amplifications of genomic DNA within well defined and demarcated limits on human chromosomes is done at a resolution of human chromosome banding limited to 400-550 bands by the technique of Comparative Genomic Hybridization (CGH). The present invention applies custom protocols to obtain human template chromosomes that are resolved to 850 to 1000 band resolution of human chromosomes (ISCN, 1985), to perform CGH on a large number of cell lines/tissue samples/tumor cells. This allows the identification of regions of genomic DNA amplifications ranging from 2-5 Mbp at the highest limits of resolution of human chromosomes, detected by fluorescent intensity evaluations performed at the microscope. Amplicons, or regions of interest, from 10-20 Mb and more are also defined by these methods. These amplicons contain a gene, or genes, that are amplified (meaning copy number gains), and/or differentially expressed in the tissue/cells of origin. Genes identified as being amplified and/or over-expressed provide targets for intervention with a small molecular therapeutic, antibodies, anti-sense or other therapeutic modalities. A gene or genes within these regions could also be used for diagnostic or prognostic molecular pathology characterization and useful as pharmacodynamic biomarkers for drug response profiling and patient sub-set selection and stratification.

### BRIEF SUMMARY OF THE INVENTION

In one aspect the present invention relates to a set of genes that have been localized within human chromosomal regions of interest (ROI) that have been identified by molecular cytogenetic techniques. In particular, the present invention relates to chromosomal regions of interest, or amplicons, that are summarized in Table 1 and containing genes corresponding to cDNA sequences shown in the sequence listing described herein.

In another aspect, the present invention relates to a method for diagnosing the presence of a cancerous condition, or diagnosing a predisposition to developing a cancerous condition, in an animal, especially a human being, by determining the amplification and/or over-expression, of one or more genes corresponding to SEQ ID NO: 1-3049 in a cell, or tissue sample, obtained from an animal. The animal may be afflicted with, or at risk of developing, such a cancerous condition, or otherwise predisposed to develop such a condition.

In a further aspect, the present invention relates to a method for the treatment of a cancerous condition, especially one involving breast, colon, lung, cervix, kidney, pancreas and prostate tissues, utilizing selected chemical agents having anti-tumor activity as identified using one of the assays disclosed herein.

Thus, in one aspect the present invention relates to a method for identifying an antineoplastic agent, comprising:

(a) contacting a test compound with a cell that expresses at least one gene corresponding to a polynucleotide comprising a nucleotide sequence of SEQ ID NO: 1-3049 and under conditions promoting expression of said gene; and

(b) determining a change in expression of said gene as a result of said contacting

wherein a change in expression indicates gene modulation thereby identifying said test compound as a gene modulating agent. In a preferred embodiment thereof, the change in expression is a decrease in expression.

In a further aspect, the present invention relates to a method for identifying a compound as an anti-neoplastic agent, comprising:

(a) contacting a test compound with a polypeptide encoded by a gene selected from SEQ ID NO: 1-3049,

(b) determining a change in a biological activity of said polypeptide due to said contacting,

wherein a change in activity indicates anti-neoplastic activity and thereby identifies such test compound as an agent having antineoplastic activity.

Preferably, the change in biological activity is a decrease in biological activity. Also preferred is where the biological activity is an enzyme activity, most preferably involving an enzyme selected from kinase, protease, peptidase, phosphodiesterase, phosphatase, dehydrogenase, reductase, carboxylase, transferase, deacetylase and polymerase. Also preferred is a biological activity that is a membrane transport activity, an integrin, a Cytochrome P450 enzyme, a nuclear hormone receptor, or a receptor activity, such as a G-protein-coupled receptor. In other preferred embodiments, the polypeptide is contained in a cell.

The present invention also relates to a method for treating cancer comprising contacting a cancerous cell with an agent first identified as having gene modulating activity using any of the methods of the invention and in an amount effective to cause a reduction in cancerous activity of said cell. In a preferred embodiment, said cancerous cell is contacted in vivo, as where the agent is administered to a mammal, especially a human being, afflicted with cancer and in an amount sufficient to ameliorate the cancer.

The present invention further relates to a method for treating cancer comprising contacting a cancerous cell with an agent having affinity for an expression product of a gene corresponding to a polynucleotide comprising a nucleotide sequence of SEQ ID NO: 1-3049 and in an amount effective to cause a reduction in cancerous activity of said cell. Preferably, the expression product is a polypeptide and the agent is an antibody.

The present invention also relates to a method for monitoring the progress of cancer therapy in a patient comprising monitoring in a patient undergoing cancer therapy the expression of a gene corresponding to a polypeptide having a sequence selected from SEQ ID NO: 1-3049, preferably wherein the gene comprises a sequence of SEQ ID NO: 1-3049, such as where the cancer therapy is chemotherapy.

In a further embodiment, the present invention relates to a method for determining the likelihood of success of cancer therapy in a patient, comprising monitoring in a patient undergoing cancer therapy the expression of a gene corresponding to a polynucleotide having a sequence of one or SEQ ID NO: 1-3049 wherein a decrease in said expression prior to completion of said cancer therapy is indicative of a likelihood of success of said cancer therapy, preferably wherein the gene comprises a sequence of SEQ ID NO: 1-3049 and wherein the cancer therapy is chemotherapy.

The present invention still further relates to a method for determining the progress of a treatment for cancer in a patient afflicted therewith, following commencement of a cancer treatment on said patient, comprising:

(a) determining in said patient a change in expression of one or more genes corresponding to a polynucleotide comprising a nucleotide sequence of SEQ ID NO: 1-3049; and

(b) determining a change in expression of said gene compared to expression of said one or more determined genes prior to commencement of said cancer treatment;

wherein said change in expression indicates progress of said treatment thereby determining the progress of said treatment. Preferred embodiments include where the change in expression is a decrease in expression and said decrease indicates success of said treatment.

### DEFINITIONS

As used herein, the following terms have the indicated definition unless expressly stated otherwise.

The term “amplicon” refers to regions of interest, i.e., genomic segments of DNA within human chromosomes in diseased states like cancer that are demarcated and limited within specific chromosomal bands. Since these amplicons contain sequences of a gene/or genes that are amplified (copy number gains), and/or differentially expressed in the tissue/cells of origin, a listing of these genes within the amplicons detected are listed in Table 3. Genes identified as being amplified and/or over-expressed within the amplicons provide a useful target for intervention with small/large molecule/protein/antibody therapeutics, anti-sense or other therapeutic modalities. A gene or genes within these regions is also useful for diagnostic or prognostic molecular pathology characterization/companion diagnostics, and useful as pharmacodynamic biomarkers for drug response profiling and patient sub-set selection and stratification.

The term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:

Percent Identity=100 [1−(C/R)]

wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. When used in relation to a polynucleotide, such terms refer to the products produced by treatment of said polynucleotides with any of the common endonucleases, or any stretch of polynucleotides that could be synthetically synthesized.

As used herein, the term “DNA segment” or “DNA sequence” refers to a DNA polymer, in the form of a separate fragment or as a component of a larger DNA construct, which has been derived from DNA, and may include both single stranded and duplex sequences. Such segments are provided in the form of an open reading frame uninterrupted by internal non-translated sequences, or introns, which are typically present in eukaryotic genes.

The term “coding region” refers to that portion of a gene which either naturally or normally codes for the expression product of that gene in its natural genomic environment, i.e., the region coding in vivo for the native expression product of the gene.

The term “nucleotide sequence” refers to a heteropolymer of deoxyribonucleotides. Generally, DNA segments encoding the proteins provided by this invention are assembled from cDNA fragments and short oligonucleotide linkers, or from a series of oligonucleotides, to provide a synthetic gene which is capable of being expressed in a recombinant transcriptional unit comprising regulatory elements derived from a microbial or viral operon.

The term “expression product” means that polypeptide or protein that is the natural translation product of the gene and any nucleic acid sequence coding equivalents resulting from genetic code degeneracy and thus coding for the same amino acid(s).

The term “fragment,” when referring to a coding sequence, means a portion of DNA comprising less than the complete coding region whose expression product retains essentially the same biological function or activity as the expression product of the complete coding region.

### DETAILED SUMMARY OF THE INVENTION

The present invention relates to a set of genes that are amplified and/or over-expressed genes in cancer cell lines and have been localized to various chromosomal regions of interest. These genes have been identified through a combination of CGH, SKY, expression analysis and Reverse Transcriptase-Polymerase Chain Reaction (RT-PCR). Such genes are both markers and potential therapeutic targets for cancer, in particular breast; colon, lung and prostate malignancies. In addition, the amplified nature of such genes provides a means of diagnosing a cancerous condition, or predisposition to a cancerous conditions, by determining the amplification of one or more of such genes in a patient afflicted with, or predisposed toward, or otherwise at risk of developing, cancer.

In one aspect the present invention relates to a set of genes that have been localized within human chromosomal regions of interest (ROI) that have been identified by molecular cytogenetic techniques. In particular, the present invention relates to chromosomal regions of interest, or amplicons, that are summarized in Table 1. Table 2 lists tissues where the amplicons are found, cell lines expressing them, the amplification ratios found in those tissues for cancer versus normal cells, amplicon size and the chromosomal locations of the amplicons. Table 3 lists the chromosomal locations and accession number identifications of these regions of interest and which serve to correlate amplicons with the cDNA sequences of SEQ ID NO: 1-3049.

TABLE 1
List of Amplicons

| AMPLICON | CHR | BPSTART | BPEND | BPLENGTH |
|---|---|---|---|---|
| A1 | 8 | 122000000 | 127500000 | 5500000 |
| A2 | 13 | 96500000 | 100000000 | 3500000 |
| A3 | 5 | 175000000 | 181500000 | 6500000 |
| A4 | 13 | 26500000 | 34000000 | 7500000 |
| A5 | 7 | 101000000 | 106000000 | 5000000 |
| A6 | 10 | 73500000 | 82500000 | 9000000 |
| A7 | 7 | 71000000 | 77500000 | 6500000 |
| A8 | 1 | 116500000 | 120000000 | 3500000 |
| A9 | 6 | 36000000 | 41000000 | 5000000 |
| A10 | 18 | 70500000 | 76500000 | 6000000 |
| A11 | 9 | 9000000 | 18500000 | 9500000 |

For Table 1, CHR means chromosome number, BPLENGTH represents the number of nucleotides in the amplicon. BPSTART refers to “base pair start point” and BPEND refers to “base pair end point” along the chromosome based on the July 2003 human reference sequence UCSC version hg16 (NCBI Build 34).

TABLE 2
Amplicon Locations

| | | | | | band | | amplicon |
|---|---|---|---|---|---|---|---|
| cell line | Amp # | tissue | chrom | band start | stop | Ratio | size_MB |
| HCC1954 | A1 | Breast | 8 | q24.13 | q24.13 | 14 | 5.3 |
| NCI_H446 | A1 | scLung | 8 | q24.13 | q24.21 | 8 | 8.3 |
| NCI_H827 | A1 | scLung | 8 | q24.13 | q24.21 | 6 | 8.3 |
| HCC202 | A1 | Breast | 8 | q24.13 | q24.21 | 6 | 8.3 |
| NCI_H82 | A1 | scLung | 8 | q24.13 | q24.13 | 7 | 5.3 |
| NCI_H23 | A1 | nscLung | 8 | q24.13 | q24.13 | 7 | 5.3 |
| MDA_MB436 | A2 | Breast | 13 | q32.2 | q32.3 | 6 | 5.3 |
| NCI_H1963 | A2 | scLung | 13 | q32.3 | q32.3 | 6 | 3.3 |
| EFM192A | A2 | Breast | 13 | q32.3 | q34 | 8 | 18.8 |
| MDA_MB157 | A2 | Breast | 13 | q32.3 | q34 | 5 | 18.8 |
| HCC1937 | A2 | Breast | 13 | q32.3 | q32.3 | 4 | 3.3 |
| SKBR3 | A2 | Breast | 13 | q32.3 | q32.3 | 4 | 18.8 |
| NCI_H1963 | A2 | nscLung | 13 | q32.3 | q32.3 | 6 | 3.3 |
| HCC1954 | A3 | Breast | 5 | q35.3 | q35.3 | 4 | 4.3 |
| MDA_MB436 | A3 | Breast | 5 | q35.1 | q35.3 | 7 | 14 |
| BT20 | A4 | Breast | 5 | q35.1 | q35.3 | 4 | 14 |
| KPL1 | A5 | Breast | 5 | q35.1 | q35.3 | 4 | 14 |
| HCC3153 | A6 | Breast | 5 | q35.3 | q35.3 | 3 | 4.3 |
| HT29 | A4 | Colon | 13 | q12.3 | q13.2 | 5 | 9 |
| SW403 | A4 | Colon | 13 | q21.1 | q21.2 | 15 | 6 |
| BT20 | A4 | Breast | 13 | q12.3 | q13.2 | 4 | 9 |
| CPDR9 | A4 | Prostate | 13 | q12.2 | q12.3 | 2 | 7.1 |
| SW480 | A5 | Colon | 7 | q22.2 | q22.2 | 9 | 1 |
| X71 | A5 | Colon | 7 | q22.1 | q22.2 | 5 | 7.2 |
| X72 | A5 | Colon | 7 | q22.3 | q22.3 | 6 | 3.3 |
| Lovo | A6 | Colon | 7 | q22.1 | q22.2 | 5 | 7.2 |
| X1819_1 | A7 | Colon | 7 | q22.1 | q22.2 | 5 | 7.2 |
| EFM19 | A6 | Breast | 10 | q22.1 | q22.3 | 6 | 15.3 |
| PC3 | A6 | Prostate | 10 | q22.2 | q22.3 | 7 | 8.3 |
| MDA_MB436 | A6 | Breast | 10 | q22.1 | q22.2 | 3 | 10.7 |
| SKBR3 | A6 | Breast | 10 | q22.2 | q22.3 | 4 | 8.3 |
| SW48 | A6 | Colon | 10 | q22.1 | q22.3 | 4 | 15.3 |
| X71 | A6 | Colon | 10 | q22.2 | q22.3 | 2 | 8.3 |
| SKBR3 | A7 | Breast | 7 | q11.23 | q11.23 | 5 | 4 |
| X72 | A7 | Colon | 7 | q11.23 | q11.23 | 7 | 4 |
| X71 | A7 | Colon | 7 | q11.23 | q11.23 | 5 | 4 |
| X1819_1 | A7 | Colon | 7 | q11.23 | q11.23 | 4 | 4 |
| NCI_H69 | A7 | scLung | 7 | q11.23 | q11.23 | 4 | 4 |
| BT20 | A8 | Breast | 1 | p12.2 | p13.2 | 10 | 9 |
| CAMA-1 | A8 | Breast | 1 | p12 | p12 | 6 | 6.7 |
| KPL-1 | A8 | Breast | 1 | p11.2 | p13.3 | 11 | 14.7 |
| Colo205 | A9 | Colon | 6 | p21.2 | p21.2 | 8 | 3.4 |
| MDA_MB231 | A9 | Breast | 6 | p21.1 | p21.2 | 7 | 9.8 |
| NCI_{——}H522 | A9 | nscLung | 6 | p21.2 | p21.31 | 6 | 9.1 |
| PANC-1 | A10 | Pancreas | 18 | q23 | q23 | 7 | 5.2 |
| NCI_H1607 | A11 | scLung | 9 | p22.2 | p23 | 10 | 14.5 |
| NCI_H446 | A11 | scLung | 9 | p22.3 | p22.3 | 8 | 2.9 |
| HCC1954 | A11 | Breast | 9 | p22.2 | p23 | 10 | 14.5 |

In addition, SEQ ID NO: 1-3049 represents the nucleotide sequences for cDNA sequences corresponding to genes located in these regions of interest. Such regions contain genes found to be amplified and over-expressed in cancerous tissues, especially of breast, colon, lung, cervix, kidney, pancreas and prostate.

Each amplicon may contain about 75 genes, at least one of which will be amplified in a cancerous condition. Genes that show amplification and/or over-expression can be indicative of the cancerous status of a given cell.

Briefly, the procedures used to identify the genes disclosed herein may be summarized as follows:

For CGH analysis, based on detailed molecular cytogenetic characterizations, the following data sets are generated, which may include regions reported in the public domain as well as unique regions not previously known.
1. A map of chromosomal regions involved in consistent, recurrent and high level genomic gains (i.e., amplifications) for a representative cancer cell line or tumor type (e.g. colon, prostate, breast and lung) that can be recognized as a pattern/signature for a given tumor type.
2. A map of chromosomal regions containing genomic losses (i.e., deletions) in each tumor type and individual cell line to be examined.
3. Levels of intensities of gains and losses categorized for entry into a database.
4. A comparison of the patterns of gains and losses between the clinical samples (e.g. colon xenografts) and cell lines (e.g., colon) of matched Stages and Grades.
5. A comparison of the patterns of gains and losses between primary prostate tumor cell lines (e.g., CPDR lines) and metastatic prostate tumor cell lines (e.g., DU 145, PC3 and LNCaP).

In accordance with the present invention, for SKY analysis, data sets were generated according to the following steps:
1. Identification and development of a database of novel chromosomal rearrangements in epithelial cancer cell lines.
2. Identification of novel translocations involving specific chromosomes or chromosomal regions
3. Reconciliation of SKY and CGH analysis on the same cell line as a verification of the combined findings.

Combining genomic DNA analysis of gains and losses in the tumor cell lines/clinical samples with cDNA expression analysis from matched tumor types displayed ordered on the assembled Human genome sequence:
1. A pattern of gene expression on a Affymetrix chip set (U95 and U133) was used to generate differential gene expression profiles between samples sets containing normal and malignant tissues from colon, prostate, lung, breast and various cell lines.
2. A Spoffire™ visualization tool was developed that allowed the generation of a list of all the genes that are present in the Human genome sequence within the defined regions of gains/losses for each cell type/tumor type to identify genes to include in the HITS platform and for identification of cancer associated genes
3. The following algorithm was employed:
   i) Match chromosomal regions of amplification/gains defined by CGH with the location of genes/ESTs on an Affymetrix chip as mapped to a Human genome template.
   ii) Identify genes/ESTs over-expressed in tumor tissue compared to normal tissue in said chromosomal regions using.
   iii) Compile data on cell lines of a particular tumor type and different tumor types showing clusters of genomic gains and losses at certain chromosomal regions.
   iv) Pick BACs that span the chromosomal regions consistently gained and containing over-expressed genes in an effort to positionally clone novel cancer genes (oncogenes and genes in relevant pathways)
   v) Validate the identified genes by
      A) Picking STS markers that identify the gene sequence and quantify the relative copy number in genomic DNA and RNA across a panel of tumor cell lines.
      B) Develop probes for FISH on chromosomes from tumor cell lines and primary tumor tissue micro-arrays.
4. The expression data from tumor cell lines that have undergone SKY/CGH analysis was used to pick candidate genes to validate as individual targets in functional genomic assays and in-vivo assays and for use in the transcriptional assay platform.

In accordance with the present invention, over-expression of cellular genes is conveniently monitored in model cellular systems using cell lines (such as is used in the example below), primary cells, or tissue samples maintained in growth media. For different purposes, these may be treated with compounds at one or more different concentrations to assay for modulating agents. Thus, cellular RNAs are isolated from the cells or cultures as an indicator of selected gene expression. The cellular RNAs are then divided and subjected to analysis to determine the presence and/or quantity of specific RNA transcripts, which transcripts are then amplified for detection purposes using standard methodologies, such as reverse transcriptase polymerase chain reaction (RT-PCR). The levels of specific RNA transcripts, including their presence or absence, are determined. When used for identification of modulating agents, such as anti-neoplastic agents, a metric is derived for the type and degree of response of the treated sample compared to control samples.

In accordance with the foregoing, the amplicons identified as being amplified and/or over-expressed, which can include increased copy number thereof, in cancerous cells are localized in chromosomal regions of interest as identified in Tables 2 and 3.

The genes localized in these amplicons may be utilized to characterize, the cancerous, or non-cancerous, status of cells, or tissues. The methods of the invention may be used with a variety of cell lines or with primary samples from tumors maintained in vitro under suitable culture conditions for varying periods of time, or in situ in suitable animal models.

The amplicons disclosed herein are expressed at levels in cancer cells that are different from the expression levels in non-cancer cells. Expression in cancer versus non-cancer cells of the same tissue type is a key identifier.

In accordance with the forgoing, the present invention also relates to a method for identifying a gene modulating agent, such as an anti-neoplastic agent, comprising:

(a) contacting a test compound, a compound whose gene-modulating and/or anti-neoplastic activity is to be determined, with one or more cells expressing one or more genes mapped to the chromosomal region of interest, or amplicon, for genes as identified in Table 3, and

(b) determining a change in expression of said one or more genes compared to when said contacting has not occurred,

wherein a change in expression of said gene is indicative of gene modulating activity, thereby identifying said test compound as a gene modulating agent.

In accordance with the foregoing, the present invention relates to a method for identifying an antineoplastic agent, comprising:

(a) contacting a test compound with a cell that expresses one or more amplicons of Table 2 having an amplification ratio of at least 2.0; and

(b) determining a change in said amplification ratio due to said contacting;

wherein a change in said amplification ratio due to said contacting indicates that said test compound has gene modulating activity

thereby identifying said test compound as a gene modulating agent.

The present invention also contemplates a method for identifying an antineoplastic agent, comprising:

(a) contacting a test compound with a cell that expresses at least one gene corresponding to a polynucleotide comprising a nucleotide sequence of SEQ ID NO: 1-3049 and under conditions promoting expression of said gene; and

(b) determining a change in expression of said gene as a result of said contacting

wherein a change in expression indicates gene modulation thereby identifying said test compound as a gene modulating agent.

In preferred embodiments of these methods, the change in expression is a decrease in expression and/or the decrease in expression is a decrease in copy number of the gene and/or the gene comprises a nucleotide sequence of one of SEQ ID NO: 1-3049 and/or the cell was genetically engineered to express said gene.

Because the genes disclosed herein are over-expressed and relate to the cancerous condition of a cell, successful anti-neoplastic activity will commonly be exhibited by agents that reduce the expression of said genes In one embodiment thereof, the change in expression is a decrease in copy number of the gene or genes under study. In accordance therewith, said change in gene copy number is conveniently determined by detecting a change in expression of messenger RNA encoded by said gene sequence. In another preferred embodiment, expression is determined for more than one such gene, such as 2, 5, 10 or more of the genes.

Thus, the present invention also encompasses a method for detecting the cancerous status of a cell, comprising detecting elevated expression in said cell of at least one gene corresponding to a polynucleotide comprising a nucleotide sequence of SEQ ID NO: 1-3049 whereby such elevated expression is indicative of cancerous status of the cell. In preferred embodiments thereof, the elevated expression is an elevated copy number of the gene.

Other methods useful in measuring a change in expression of the genes disclosed herein include measuring a change in the amount or rate of synthesis of a polypeptide encoded by said gene, preferably a decrease in synthesis of said polypeptide. Most preferably, the polypeptide comprises an amino acid sequence highly homologous to a sequence encoded by a gene mapping to an amplicon disclosed herein and whose expression is elevated in cancer.

The methods of the invention can thus be utilized to identify anti-neoplastic agents useful in treatment of cancerous conditions. Such activity can be further modified by first identifying such an agent using an assay as already described and further contacting such agent with a cancerous cell, followed by monitoring of the status of said cell, or cells. A change in status indicative of successful anti-neoplastic activity may include a decrease in the rate of replication of the cancerous cell(s), a decrease in the total number of progeny cells that can be produced by said cancerous cell(s), or a decrease in the number of times said cancerous cell(s) can replicate, or the death of said cancerous cell(s).

Anti-neoplastic agents may also be identified using recombinant cells suitably engineered to contain and express the cancer-related genes disclosed herein. In one such embodiment, a recombinant cell is formed using standard technology and then utilized in the assays disclosed herein. Methods of forming such recombinant cells are well known in the literature. See, for example, Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), and Recombinant Gene Expression Protocols, in Methods in Molecular Biology, Vol. 62, (Tuan, ed., Humana Press, Totowa, N.J., 1997), the disclosures of which are hereby incorporated by reference.

The present invention also relates to a method for detecting the cancerous status of a cell, comprising detecting elevated copy number and/or expression in said cell of at least one gene that maps to a chromosomal region of interest, or amplicon, as identified in Table 3. Such elevated expression may be readily monitored by comparison to that of otherwise normal cells having the same genes. Elevated expression of such genes is indicative of the cancerous state. Such elevated expression, including increased copy number, may be the expression of more than one such gene.

The present invention also relates to a method for detecting a cancer-linked gene comprising the steps of contacting a test compound, identified as having gene modulating activity for a gene mapping to one of the amplicons disclosed herein, with a cell expressing a test gene and detecting modulation, such as decreased activity, of such test gene relative to when said compound is not present thereby identifying said test gene as a cancer-related gene. In preferred embodiments, the gene determined by said method is an oncogene, or cancer facilitating gene.

In another embodiment, there is provided a method for treating cancer comprising contacting a cancerous cell with an agent first identified as having gene modulating activity using any of the assay methods disclosed according to the invention and in an amount effective to reduce the cancerous activity of said cell. In a preferred embodiment, the cancerous cell is contacted in vivo. In other preferred embodiments, said reduction in cancerous activity is a decrease in the rate of proliferation of said cancerous cell, or said reduction in cancerous activity is the death of said cancerous cell.

The present invention further relates to a method for treating cancer comprising contacting a cancerous cell with an agent having activity against an expression product encoded by a gene mapping to an amplicon as disclosed herein, preferably where the expression product is a polypeptide. In a preferred embodiment, said cancerous cell is contacted in vivo. In another preferred embodiment, the agent is an antibody.

Nucleotide sequences mapping to the amplicons disclosed herein may be genomic in nature and thus represent the sequence of an actual gene, such as a human gene, or may be a cDNA sequence derived from a messenger RNA (mRNA) and thus represent contiguous exonic sequences derived from a corresponding genomic sequence or they may be wholly synthetic in origin for purposes of testing. Such cDNA sequences, mapping to the amplicons disclosed herein are identified as SEQ ID NO: 1-3049.

As described in the Example below, the expression of cancer-related genes may be determined from the relative expression levels of the RNA complement of a cancerous cell relative to a normal (i.e., non-cancerous) cell. Because of the processing that may take place in transforming the initial RNA transcript into the final mRNA, the sequences disclosed herein may represent less than the full genomic sequence. They may also represent sequences derived from ribosomal and transfer RNAs. Consequently, the genes present in the cell (and representing the genomic sequences) and the sequences disclosed in SEQ ID NO: 1-3049, which are mostly cDNA sequences, may be identical or may be such that the cDNAs contain less than the full genomic sequence. Such genes and cDNA sequences are still considered corresponding sequences because they both encode similar RNA sequences. Thus, by way of non-limiting example only, a gene that encodes an RNA transcript, which is then processed into a shorter mRNA, is deemed to encode both such RNAs and therefore encodes an RNA complementary to (using the usual Watson-Crick complementarity rules), or that would otherwise be encoded by, a cDNA (for example, a sequence as disclosed herein). Thus, the sequences disclosed herein correspond to genes contained in the cancerous or normal cells used to determine relative levels of expression because they represent the same sequences or are complementary to RNAs encoded by these genes. Such genes also include different alleles and splice variants that may occur in the cells used in the methods of the invention.

In addition, sequences encoding the same proteins as any of these genes, regardless of the percent identity of such sequences, are also specifically contemplated by any of the methods of the present invention that rely on any or all of said sequences, regardless of how they are otherwise described or limited. Thus, any such sequences are available for use in carrying out any of the methods disclosed according to the invention. Such sequences also include any open reading frames, as defined herein, present within any genes mapping to the amplicons of the invention.

The present invention also finds use as a means of diagnosing the presence of cancer in a patient, as where a sample of cancerous tissue or cells, or tissues or cells suspected of being cancerous, are examined for elevated expression, such as at least 2 fold expression, of a gene in one of the amplicons disclosed herein, such as an increased expression of a cDNA sequence, or polypeptide encoded by said cDNA sequence, disclosed in Table 3 and being one of the sequences of SEQ ID NC): 1-3049.

For such purposes, and in accordance with the disclosure elsewhere herein, such diagnosis is based on the detection of elevated expression or amplification, such as elevated copy number, of one or more of the genes identified according to the invention. Such elevated expression can be determined by any of the means described herein.

In one such embodiment, the elevated expression, as compared to normal cells and/or tissues of the same organ, is determined by measuring the relative rates of transcription of RNA, such as by production of corresponding cDNAs and then analyzing the resulting DNA using probes developed from genes mapping to the amplicons of the invention. Thus, the levels of cDNA produced by use of reverse transcriptase with the full RNA complement of a cell suspected of being cancerous produces a corresponding amount of cDNA that can then be amplified using polymerase chain reaction, or some other means, such as rolling circle amplification, to determine the relative levels of resulting cDNA and, thereby, the relative levels of gene expression.

For RNA analysis, the latter may be isolated from samples in a variety of ways, including lysis and denaturation with a phenolic solution containing a chaotropic agent (e.g., triazol) followed by isopropanol precipitation, ethanol wash, and resuspension in aqueous solution; or lysis and denaturation followed by isolation on solid support, such as a Qiagen resin and reconstitution in aqueous solution; or lysis and denaturation in non-phenolic, aqueous solutions followed by enzymatic conversion of RNA to DNA template copies. Steady state RNA levels for a given type of cell or tissue may have to be ascertained prior to employment of the methods of the invention but such is well within the skill of those in the art and will not be further described in detail herein.

Alternatively, increased expression, such as increased copy number, may be determined for the genes present in a cancerous cell, or a cell suspected of being cancerous, by determining elevated expression within the regions of interest, or amplicons, disclosed herein. Thus, the DNA of such cells may be extracted and probed for increased gene expression within the area disclosed herein as amplified in different cancer types and tissues.

In employing the methods of the invention, it should be borne in mind that gene expression indicative of a cancerous state need not be characteristic of every cell found to be cancerous. Thus, the methods disclosed herein are useful for detecting the presence of a cancerous condition within a tissue where less than all cells exhibit the complete pattern of over-expression. For example, a set of selected genes, which are found within the regions of interest disclosed herein, may be found, using appropriate probes, either DNA or RNA, to be present in as little as 60% of cells derived from a sample of tumorous, or malignant, tissue while being absent from as much as 60% of cells derived from corresponding non-cancerous, or otherwise normal, tissue (and thus being present in as much as 40% of such normal tissue cells). In a preferred embodiment, such gene pattern is found to be present in at least 70% of cells drawn from a cancerous tissue and absent from at least 70% of a corresponding normal, non-cancerous, tissue sample. In an especially preferred embodiment, such gene pattern is found to be present in at least 80% of cells drawn from a cancerous tissue and absent from at least 80% of a corresponding normal, non-cancerous, tissue sample. In a most preferred embodiment, such gene pattern is found to be present in at least 90% of cells drawn from a cancerous tissue and absent from at least 90% of a corresponding normal, non-cancerous, tissue sample. In an additional embodiment, such gene pattern is found to be present in at least 100% of cells drawn from a cancerous tissue and absent from at least 100% of a corresponding normal, non-cancerous, tissue sample, although the latter embodiment may represent a rare occurrence.

Because changes in expression of these genes (up-regulation) are linked to the disease state (i.e. cancer), the change in expression may contribute to the initiation or progression of the disease. For example, if a gene that is up-regulated is an oncogene such a gene provides for a means of screening for small molecule therapeutics beyond screens based upon expression output alone. For example, genes that display up-regulation in cancer and whose elevated expression contributes to initiation or progression of disease represent targets in screens for small molecules that inhibit or block their function. Examples include, but are not be limited to, kinase inhibition, cellular proliferation, substrate analogs that block the active site of protein targets, etc.

It should be noted that there are a variety of different contexts in which genes have been evaluated as being involved in the cancerous process. Thus, some genes may be oncogenes and encode proteins that are directly involved in the cancerous process and thereby promote the occurrence of cancer in an animal. Other genes may simply be involved either directly or indirectly in the cancerous process or condition and may serve in an ancillary capacity with respect to the cancerous state. All such types of genes are deemed with those to be determined in accordance with the invention as disclosed herein. Thus, the gene determined by said method of the invention may be an oncogene, or the gene determined by said method may be a cancer facilitating gene, the latter including a gene that directly or indirectly affects the cancerous process, either in the promotion of a cancerous condition or in facilitating the progress of cancerous growth or otherwise modulating the growth of cancer cells, either in vivo or ex vivo. Such genes may work indirectly where their expression alters the activity of some other gene or gene expression product that is itself directly involved in initiating or facilitating the progress of a cancerous condition. For example, a gene that encodes a polypeptide, either wild or mutant in type, which polypeptide acts to suppress of tumor suppressor gene, or its expression product, will thereby act indirectly to promote tumor growth.

Many cancerous genes appear to have their effect by encoding an aberrant protein that functions in a cell in a manner different from that of normal cells, or else said protein is overproduced or underproduced as a result of some mutation in the coding sequence, or promoter or enhancer sequences, of a particular gene, such as one of Genes 1-3049 disclosed herein and expressed by the amplicons of the invention.

In accordance with the present invention, there are provided methods for measuring the activity, such as a biological activity, of such a polypeptide. Such biological activity may include any measurable activity, such as chemical reactivity, catalytic ability, binding to specific structures and receptors, acting as a receptor, or just being present in a membrane of a cell and therefore available as a target site for antibodies or other agents. Any such polypeptides may thus provide a target for a chemotherapeutic agent, especially an antineoplastic agent.

As is well known in the art, polypeptide activities can be measured in different ways so as to enable screening procedures for agents, such as test compounds, that inhibit the activity of the polypeptide and thereby work against the function of that polypeptide, such as where the polypeptide is some type of cancer-related protein, such as that produced by expression of an oncogene, or where the polypeptide is overproduced as part of the cancer initiating or facilitating process. As non-limiting examples, such screening methods for antineoplastic agents can include the measurement of compounds that bind to proteins (or that bind to a gene or a transcript of a gene), compounds that inhibit expression (including processing and/or maturation) of a protein, or the detection of downstream reaction product, most often with specific antibodies using enzyme-linked immunosorbent assay (ELISA) procedures well known in the art, or compounds that inhibit activity, such as enzyme activity or some other function, or compounds that interact with upstream or downstream proteins (such as with transcription factors or other binding proteins that may serve to regulate gene expression).

In accordance with the foregoing, the present invention relates to a method for identifying a compound as an anti-neoplastic agent, comprising:

(a) contacting a test compound with a polypeptide encoded by a gene selected from SEQ ID NO: 1-3049,

(b) determining a change in a biological activity of said polypeptide due to said contacting,

wherein a change in activity indicates anti-neoplastic activity and thereby identifies such test compound as an agent having antineoplastic activity.

In a preferred embodiment, the change in biological activity is a decrease in biological activity.

In another preferred embodiment, the biological activity is an enzyme activity, such as where the enzyme is one selected from the group kinase, protease, peptidase, phosphodiesterase, phosphatase, dehydrogenase, reductase, carboxylase, transferase, deacetylase and polymerase.

Assays for these enzymes are available, such as for phosphodiesterases (the most pharmacologically relevant phosphodiesterases are those that hydrolyze cyclic nucleotides (see, for example, cAMP and cGMP assays available from Perkin-Elmer, as well as Estrade et al., Eur. J. Pharmacol. 352:2-3, 157-163 (1998)).

Protein phosphatases remove phosphate residues from proteins. Most tests of their activity use the same assays as for protein kinases. A non-radioactive phosphatase assay system is available from Promega Biotech.

The therapeutically most relevant dehydrogenases oxidize or reduce small molecular weight metabolites, esp. steroid hormones, or that generally use or generate NAD or NADP (see: Haeseleer et al., J. Biol. Chem., 273:21790-21799 (1998)). A commercial assay is available from Cayman Chemical (at www.caymanchem.com).

Gamma-carboxylases are important enzymes in the blood coagulation process. The main assay protocols use synthetic peptides (see: Ulrich et al., J. Biol. Chem., 263:9697-9702 (1988); Begley et al., J. Biol. Chem., 275:36245-36249 (2000)).

In highly preferred embodiments, the kinase is one of a protein kinase, a serine or threonine kinase, or a receptor tyrosine protein kinase. Where the polypeptide encoded by a gene of the invention is a protein kinase, especially involving tyrosine kinase, various assays for activity are available. Protein kinases add phosphate groups to serine, threonine or tyrosine residues on proteins, most commonly measured with phospho-serine, threonine, or tyrosine-specific antibodies, or generation of radiolabeled substrate, or consumption of ATP, or phosphorylation of (synthetic) small peptides, or measuring downstream enzyme activity and gene transcription. Such assays are commercially available. (See, for example, the tyrosine kinase assay from Roche Molecular Biochemicals). Assays for serine/threonine kinases are also available at Chromagen.com, Upstate Biotechnology, Inc. (Lake Placid, N.Y., and at upstatebiotech.com) and from Applied BioSystems (Foster City, Calif. (home.appliedbiosystems.com)).

In other specific embodiments, the protease is a serine protease, cysteine protease or aspartic acid protease, or the transferase is a methyltransferase, preferably a cytosine methyltransferase or an adenine methyltransferase, or the deacetylase is a histone deacetylase, or the carboxylase is a γ-carboxylase, or the peptidase is a zinc peptidase, or the polymerase is a DNA polymerase or an RNA polymerase.

Proteases degrade proteins, un-specifically or at specific sites. Almost all pharmacologically relevant ones have very narrowly defined specific substrates, and their activity is most often measured by directly measuring cleavage product or generation of (fluorescent) light after cleavage of synthetic substrates. Assays are available for serine proteases (Calbiochem, Palo Alto, Calif., and see Berdichevsky et al., J. Virol. Methods, 107:245-255 (2003), for systeine proteases (See: Schulz et al., Mol. Pathol., 51:222-24 (1998) and Seizer et al., PNAS, 96:11015-11022 (1999)), for aspartic acid proteases (Geno Tech, Inc. at www.genotech.com) and for zinc peptidases (see Evans et al., J. Biol. Chem., 278:23180-23186 (2003)).

Both (regulatory) DNA-methylases and (biosynthetic) protein methylases that are drug targets. (See: Jonassen and Clarke, J. Biol. Chem., 275:12381-12387 (2000); Jackson et al., Nature, 416:556-560 (2002)).

Most HDAC (histone deacetylase) assays use colorimetric or fluorometric (synthetic) substrates. Standard assays are for binding, especially molecular size changes, blocking a specific site, and effects on transcription or downstream reactions (if DNA or RNA is the direct target of a drug). A commercial assay is available from Vinci Biochem (at www.vincibiochem.it).

In another specific embodiment, the biological activity is a membrane transport activity, preferably wherein the polypeptide is a cation channel protein, an anion channel protein, a gated-ion channel protein or an ABC transporter protein. Drug effects on the activity of transporter and channel proteins are screened by measuring increase or decrease of the ((radio-)labeled) transported entity inside or outside the cell, in cell-based assays, ATP consumption (in the case of ATPases), or changes in cell membrane potential. Assays employing such proteins are available, such as for ABC transporter (see: Marcil et al., Lancet, 354:1341-46 (1999) and for ion channels (from Evotec OAI, at www.evotecoai.com and from PharmaLinks, at www.pharmalinks.org/research/cellsignalling).

In one embodiment, the polypeptide is an integrin (the signal transduction pathways elicited by the integrins are slow and not very well characterized, hence most assays are either just binding assays or measure downstream biological phenomena (such as migration, invasion, etc.) (See: Ganta et al., Endocrinology, 138:3606-3612 (1997); Sim et al., J. Biomed. Mater. Research, 68A:352-359 (2004); and Weinreb et al., Anal. Biochem., 306:305-313 (2002)), or a Cytochrome P450 enzyme (almost all cytochrome assays require knowledge of what the substrate is and measure conversion of substrate (free or (radio-)labeled) or generation of product; useful C¹⁴-labeled substrates are available from Amersham Biosciences at www1.amershambiosciences.com), or a nuclear hormone receptor (Assays available from Discoverx, Fremont, Calif., such as an estrogen assay; also see Rosen et al., Curr. Opin. Drug. Discov. Devel., 6:224-30 (2003)).

In one preferred embodiment, the biological activity is a receptor activity, preferably where the receptor is a G-protein-coupled receptor (GPCR).

GPCRs are transmembrane proteins that wind 7 times back and forth through a cell's plasma membrane with a ligand binding site located on the outside of the membrane surface of the cell and the effector site being present inside the cell. These receptors bind GDP and GTP. In response to ligand binding, GPCRs activate signal transduction pathways which induce a number of assayable physiological changes, e.g., an increase in intracellular calcium levels, cyclic-AMP, inositol phosphate turnover, and downstream gene transcription (directly or via reporter-assays) along with other translocation assays available for measuring GPCR activation when the polypeptide encoded by a gene of the invention is a GPCR. Thus, such proteins work through a second messenger. The result is activation of CREB, a transcription factor that stimulates the production of gene products. One useful assay is the so-called BRET2/arrestin assay, useful in screening for compounds that interact with GPCRs. (See: Bertrand et al, J. Recept. Signal Transduct Res., 22:533-41 (February-November 2002)). In addition, numerous assays are commercially available, such as the Transfluor Assay, available from Norak Biosciences, Inc. (www.norakbio.com) or ArrayScan and KineticScan, both from Cellomics, or assays from CyBio (Jena, Germany).

Assays useful with the invention are usually set up to screen for agonists or antagonists after adding ligand, but effects on most of these parameters can be measured whether or not the ligand for the receptor is known. Such assays may involve radioligand-binding assays. Activation of the majority of GPCRs by agonists leads to the interaction of beta-arrestin (a protein that is involved in receptor desensitization and sequestration) with the receptor, which is measurable by fluorescence energy transfer

The disclosure of all journal articles, or other publications, referred to herein are hereby incorporated by reference in their entirety.

In one embodiment, the polypeptide is in a solution or suspension and contact with the test compound is by direct contact between the test compound and the protein molecule. Alternatively, the polypeptide may be in a cell and the test compound may have to diffuse into the cell in order to contact the polypeptide. In an alternative embodiment, the test compound may be contacted with a cell that contains or expresses the polypeptide but the test compound may have no direct contact with the polypeptide. In stead, the test compound may act to induce production and/or activity of a different compound, such as an intracellular second messenger that serves to contact the polypeptide and modulate or change the biological activity of this polypeptide.

In accordance with the foregoing, the method of the present invention includes cancer modulating agents that are themselves either polypeptides, or small chemical entities, that affect the cancerous process, including initiation, suppression or facilitation of tumor growth, either in vivo or ex vivo. Such agents may also be antibodies that react with one or more polypeptides encoded by genes present in the amplicons of the invention.

In keeping with the disclosure herein, the present invention also relates to a method for treating cancer comprising contacting a cancerous cell with an agent having activity against an expression product encoded by a gene mapping within regions of chromosomal interest.

The method of the present invention includes embodiments of the above-recited method wherein said cancer cell is contacted in vivo as well as ex vivo, preferably wherein said agent comprises a portion, or is part of an overall molecular structure, having affinity for said expression product. In one such embodiment, said portion having affinity for said expression product is an antibody.

In one embodiment of the present invention, a chemical agent, such as a protein or other polypeptide, is joined to an agent, such as an antibody, having affinity for an expression product of a cancerous cell, such as a polypeptide or protein encoded by a gene related to the cancerous process, especially a gene mapping to an amplicon as disclosed herein In a specific embodiment, said expression product acts as a therapeutic target for the affinity portion of said anticancer agent and where, after binding of the affinity portion of such agent to the expression product, the anti-cancer portion of said agent acts against said expression product so as to neutralize its effects in initiating, facilitating or promoting tumor formation and/or growth. In a separate embodiment of the present invention, binding of the agent to said expression product may, without more, have the effect of deterring cancer promotion, facilitation or growth, especially where the presence of said expression product is related, either intimately or only in an ancillary manner, to the development and growth of a tumor. Thus, where the presence of said expression product is essential to tumor initiation and/or growth, binding of said agent to said expression product will have the effect of negating said tumor promoting activity. In one such embodiment, said agent is an apoptosis-inducing agent that induces cell suicide, thereby killing the cancer cell and halting tumor growth.

Many cancers contain chromosomal rearrangements, which typically represent translocations, amplifications, or deletions of specific regions of genomic DNA. A recurrent chromosomal rearrangement that is associated with a specific stage and type of cancer always affects a gene (or possibly genes) that play a direct and critical role in the initiation or progression of the disease. Many of the known oncogenes or tumor suppressor genes that play direct roles in cancer have either been initially identified based upon their positional cloning from a recurrent chromosomal rearrangement or have been demonstrated to fall within a rearrangement subsequent to their cloning by other methods. In all cases, such genes display amplification at both the level of DNA copy number and at the level of transcriptional expression at the mRNA level.

In accordance with the present invention, said functionally related genes are genes modulating the same metabolic pathway or said genes are genes encoding functionally related polypeptides. In one such embodiment, said genes are genes whose expression is modulated by the same transcriptional activator or enhancer sequence, especially where said transcriptional activator or enhancer increases, or otherwise modulates, the activity of a gene mapping to one of the amplicons of the invention.

The present invention also relates to a process that comprises a method for producing a product, such as test data, comprising identifying an agent according to one of the disclosed methods for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

In accordance with the foregoing, the present invention relates to a method for producing test data with respect to the anti-neoplastic activity of a compound, such as a test compound as defined herein, comprising:

(a) identifying a test compound as having anti-neoplastic activity using a method of the invention, such as measuring the biological activity of a polypeptide encoded by a gene of Table 3 (SEQ ID NO: 1-3049), and

(b) producing test data with respect to the anti-neoplastic activity of said test compound sufficient to identify the chemical structure of said test compound.

In another embodiment, the present invention provides a method for monitoring the progress of a cancer treatment, such as where the methods of the invention permit a determination that a given course of cancer therapy is or is not proving effective because of an increased or decreased expression of a gene, or genes, mapping to an amplicon as disclosed herein. For example, where there is an increased copy number of one or more of said genes monitoring of such genes can predict success or failure of a course of therapy, such as chemotherapy, or predict the likelihood of a relapse based on elevated activity or expression of one or more of these genes following such course of therapy.

In accordance with the foregoing, the present invention contemplates a method for determining the progress of a treatment for cancer in a patient afflicted with cancer, following commencement of a cancer treatment on said patient, comprising determining in said patient a change in expression of one or more genes, preferably more than one, corresponding to a gene of Table 3 or encoding a polypeptide or transcript of such a gene, or genes compared to expression of said one or more determined genes prior to commencement of said cancer treatment, wherein a change in expression, especially a decrease in expression, indicates positive effects of such treatment, thereby determining the progress of said treatment.

In a preferred embodiment, the detected change in expression is a decrease in expression. In another preferred embodiment, the cancer treatment is treatment with a chemotherapeutic agent, especially an agent that modulates, preferably decreases, expression of a gene identified herein, such as where said agent was first identified as having anti-neoplastic activity using a method of the invention. Thus, in accordance with this aspect of the present invention, a patient, or even a research animal, such as a mouse, rat, rabbit or primate, afflicted with cancer, including a cancer induced for research purposes, is introduced to a cancer treatment regimen, such as administration of an anti-cancer agent, including one first identified as having anti-neoplastic activity by one or more of the screening methods disclosed herein. The progress and success or failure of such treatment is subsequently ascertained by determining the subsequent expression of one or more, preferably at least 3, or 5, or 10, of genes mapping to one or more of the amplicons disclosed herein, preferably to the same amplicon, or that encodes a transcript or polypeptide of such a gene following said treatment. In a preferred embodiment, a treatment that reduces said expression is deemed advantageous and may then be the basis for continuing said treatment. The methods of the invention thereby provide a means of continually monitoring the success of the treatment and evaluating both the need, and desirability, of continuing said treatment. In addition, more than one said treatment may be administered simultaneously without diminishing the value of the methods of the invention in determining the overall success of such combined treatment. Thus, more than one said anti-neoplastic agent may be administered to the same patient and overall effectiveness ascertained by the recited methods.

In accordance with the foregoing, the present invention also contemplates a method for determining the likelihood of survival of a patient afflicted with cancer, following commencement of a cancer treatment on said patient, comprising determining in said patient a change in expression of one or more genes, preferably more than one, corresponding to a gene of Table 3 or encoding a polypeptide or transcript of such a gene, or genes, compared to expression of said one or more determined genes prior to commencement of said cancer treatment, wherein a change in expression, especially a decrease in expression, indicates positive and life-extending effects of such treatment, thereby determining the likelihood of survival of said treatment.

In a preferred embodiment, the detected change in expression is a decrease in expression and said determined gene, or genes, may include 2, 3, 5, 10 or more of the genes described herein. Thus, the methods of the invention may be utilized as a means for compiling cancer survival statistics following one or more, possibly combined, treatments for cancer as in keeping with the other methods disclosed herein.

The genes of the amplicons, or regions of interest, identified herein also offer themselves as pharmacodynamic markers (or as pharmacogenetic and/or surrogate markers), such as for patient profiling prior to clinical trials and/or targeted therapies, including combination treatments, resulting from the identification of these genes as valid gene targets for chemotherapy based on the screening procedures of the invention. In one embodiment thereof, the likelihood of success of a cancer treatment with a selected chemotherapeutic agent may be based on the fact that such agent has been determined to have expression modulating activity with one or more genes identified herein, especially where said genes have been identified as showing elevated expression levels in samples from a prospective patient afflicted with cancer. Methods described elsewhere herein for determining cancerous status of a cell may find ready use in such evaluations.

It should be cautioned that, in carrying out the procedures of the present invention as disclosed herein, any reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention will now be further described by way of the following non-limiting example. In applying the disclosure of the example, it should be kept clearly in mind that other and different embodiments of the methods disclosed according to the present invention will no doubt suggest themselves to those of skill in the relevant art.

### EXAMPLE

Cancerous cells that over-express one or more genes mapping to the amplicons disclosed herein, are grown to a density of 10⁵ cells/cm² in Leibovitz's L-15 medium supplemented with 2 mM L-glutamine (90%) and 10% fetal bovine serum. The cells are collected after treatment with 0.25% trypsin, 0.02% EDTA at 37° C. for 2 to 5 minutes. The trypsinized cells are then diluted with 30 ml growth medium and plated at a density of 50,000 cells per well in a 96 well plate (200 μl/well). The following day, cells are treated with either compound buffer alone, or compound buffer containing a chemical agent to be tested, for 24 hours. The media is then removed, the cells lysed and the RNA recovered using the RNAeasy reagents and protocol obtained from Qiagen. RNA is quantitated and 10 ng of sample in 1 μl are added to 24 μl of Taqman reaction mix containing 1×PCR buffer, RNAsin, reverse transcriptase, nucleoside triphosphates, amplitaq gold, tween 20, glycerol, bovine serum albumin (BSA) and specific PCR primers and probes for a reference gene (18S RNA) and a test gene (Gene X). Reverse transcription is then carried out at 48° C. for 30 minutes. The sample is then applied to a Perlin Elmer 7700 sequence detector and heat denatured for 10 minutes at 95° C. Amplification is performed through 40 cycles using 15 seconds annealing at 60° C. followed by a 60 second extension at 72° C. and 30 second denaturation at 95° C. Data files are then captured and the data analyzed with the appropriate baseline windows and thresholds.

The quantitative difference between the target and reference genes is then calculated and a relative expression value determined for all of the samples used. This procedure is then repeated for each of the target genes in a given signature, or characteristic, set and the relative expression ratios for each pair of genes is determined (i.e., a ratio of expression is determined for each target gene versus each of the other genes for which expression is measured, where each gene's absolute expression is determined relative to the reference gene for each compound, or chemical agent, to be screened). The samples are then scored and ranked according to the degree of alteration of the expression profile in the treated samples relative to the control. The overall expression of the set of genes relative to the controls, as modulated by one chemical agent relative to another, is also ascertained. Chemical agents having the most effect on a given gene, or set of genes, are considered the most anti-neoplastic.

### Sequence Listing on CD-ROM Only

The sequences disclosed herein as SEQ ID NO: 1-3049 in the sequence listing are contained on compact disc (CD-ROM) only (denoted as Filename: Avalon 237 (5,279 kB), 4 copies of which appear on discs denoted Copy 1, Copy 2, Copy 3 and CRF, and which discs were generated on 7 Mar. 2005), which accompanies this application and the contents of said CD-ROMs are hereby incorporated by reference in their entirety. These sequence numbers correspond to cDNA sequences derived from the genes identified in Table 3.

TABLE 3
Amplicon Identification

| | | | Chro- | | |
|---|---|---|---|---|---|
| Amplicon | Transcript Id | Name | mosome | bpstart | bpend |
| A1 | ENST00000303924 | HAS2 | 8 | 122582937 | 122598168 |
| A1 | ENSESTT00000046662 | t | 8 | 122585931 | 122609941 |
| A1 | ENSESTT00000046660 | | 8 | 122608522 | 122610061 |
| A1 | ENSESTT00000046661 | | 8 | 122640599 | 122653390 |
| A1 | ENST00000328524 | | 8 | 123738119 | 123738958 |
| A1 | ENSESTT00000047108 | | 8 | 123750171 | 123920219 |
| A1 | ENST00000314393 | NM_014943 | 8 | 123750577 | 123943336 |
| A1 | ENSESTT00000047109 | | 8 | 123789727 | 123790358 |
| A1 | ENSESTT00000047110 | | 8 | 123921884 | 123943336 |
| A1 | ENSESTT00000047116 | | 8 | 123983935 | 123988137 |
| A1 | ENSESTT00000047111 | | 8 | 123983935 | 124011208 |
| A1 | ENSESTT00000047112 | | 8 | 123983935 | 124011208 |
| A1 | ENST00000259512 | NM_024295 | 8 | 123984034 | 124011088 |
| A1 | ENSESTT00000047115 | | 8 | 123984041 | 123990329 |
| A1 | ENSESTT00000047114 | | 8 | 123987689 | 123999547 |
| A1 | ENSESTT00000047113 | | 8 | 123991511 | 124011208 |
| A1 | ENSESTT00000065616 | | 8 | 124041564 | 124062478 |
| A1 | ENST00000287380 | NM_145647 | 8 | 124041598 | 124120767 |
| A1 | ENSESTT00000065617 | | 8 | 124042077 | 124066211 |
| A1 | ENST00000309336 | Q8TAK7 | 8 | 124046061 | 124098030 |
| A1 | ENST00000327098 | Q8TAK7 | 8 | 124062489 | 124073028 |
| A1 | ENSESTT00000065618 | | 8 | 124074181 | 124088995 |
| A1 | ENSESTT00000065619 | | 8 | 124094878 | 124098018 |
| A1 | ENSESTT00000065620 | | 8 | 124099026 | 124120981 |
| A1 | ENSESTT00000065621 | | 8 | 124109587 | 124120981 |
| A1 | ENSESTT00000065622 | | 8 | 124109691 | 124120981 |
| A1 | ENST00000318462 | Q86UY5 | 8 | 124147875 | 124177793 |
| A1 | ENSESTT00000065624 | | 8 | 124151533 | 124162903 |
| A1 | ENSESTT00000065623 | | 8 | 124151533 | 124177809 |
| A1 | ENST00000276699 | NM_032899 | 8 | 124151685 | 124176255 |
| A1 | ENSESTT00000065671 | | 8 | 124188795 | 124210200 |
| A1 | ENST00000276704 | NM_032847 | 8 | 124188931 | 124210174 |
| A1 | ENSESTT00000065672 | | 8 | 124189023 | 124210138 |
| A1 | ENSESTT00000065673 | | 8 | 124195324 | 124210138 |
| A1 | ENSESTT00000065668 | | 8 | 124218685 | 124236123 |
| A1 | ENST00000297857 | ZHX1 | 8 | 124222153 | 124224774 |
| A1 | ENSESTT00000065670 | | 8 | 124224524 | 124243082 |
| A1 | ENSESTT00000065669 | | 8 | 124224572 | 124243107 |
| A1 | ENST00000309019 | | 8 | 124284858 | 124285232 |
| A1 | ENST00000287394 | NM_014109 | 8 | 124289962 | 124365185 |
| A1 | ENSESTT00000065666 | | 8 | 124294833 | 124315051 |
| A1 | ENSESTT00000065667 | | 8 | 124305201 | 124313890 |
| A1 | ENSESTT00000065665 | | 8 | 124315497 | 124328452 |
| A1 | ENST00000329771 | | 8 | 124369449 | 124370499 |
| A1 | NST00000287387 | M_018024 | 8 | 124385553 | 124410848 |
| A1 | NSESTT00000065625 | | 8 | 124385553 | 124410849 |
| A1 | ENSESTT00000065626 | | 8 | 124385601 | 124410849 |
| A1 | ENSESTT00000065627 | | 8 | 124385602 | 124410849 |
| A1 | ENSESTT00000065628 | | 8 | 124385602 | 124410849 |
| A1 | ENST00000287396 | FBXO32 | 8 | 124471947 | 124510034 |
| A1 | ENSESTT00000065664 | | 8 | 124472004 | 124500973 |
| A1 | ENSESTT00000065662 | | 8 | 124472004 | 124510034 |
| A1 | ENSESTT00000065663 | | 8 | 124483080 | 124510034 |
| A1 | ENST00000325995 | | 8 | 124614600 | 124621754 |
| A1 | ENST00000330051 | | 8 | 124614651 | 124621727 |
| A1 | ENST00000329589 | | 8 | 124614654 | 124621727 |
| A1 | ENSESTT00000065661 | | 8 | 124649681 | 124657768 |
| A1 | ENST00000262219 | ANXA13 | 8 | 124650068 | 124681581 |
| A1 | ENSESTT00000065659 | | 8 | 124662584 | 124706220 |
| A1 | ENSESTT00000065660 | | 8 | 124667216 | 124706214 |
| A1 | ENST00000334705 | Q8N6F3 | 8 | 124737531 | 124744075 |
| A1 | ENST00000325963 | NM_144963 | 8 | 124747485 | 124784276 |
| A1 | ENSESTT00000065657 | | 8 | 124749308 | 124767066 |
| A1 | ENSESTT00000065656 | | 8 | 124753384 | 124767066 |
| A1 | ENSESTT00000065658 | | 8 | 124777101 | 124779458 |
| A1 | ENST00000297628 | | 8 | 124934987 | 124955009 |
| A1 | ENST00000321393 | NM_73684 | 8 | 124982464 | 124984850 |
| A1 | ENSESTT00000049471 | | 8 | 125004837 | 125009297 |
| A1 | ENST00000308614 | NM_182525 | 8 | 125014902 | 125029621 |
| A1 | ENST00000330102 | | 8 | 125045027 | 125088508 |
| A1 | ENST00000327482 | | 8 | 125120753 | 125121402 |
| A1 | ENST00000297632 | Q8WVK5 | 8 | 125280819 | 125341514 |
| A1 | ENSESTT00000049470 | | 8 | 125282330 | 125296251 |
| A1 | ENST00000328599 | NM_017956 | 8 | 125419757 | 125421103 |
| A1 | ENSESTT00000049469 | | 8 | 125441637 | 125443183 |
| A1 | ENSESTT00000049445 | | 8 | 125443596 | 125455222 |
| A1 | ENST00000303545 | RNF139 | 8 | 125443702 | 125456727 |
| A1 | ENSESTT00000049468 | | 8 | 125454689 | 125456214 |
| A1 | ENSESTT00000049466 | | 8 | 125457337 | 125491832 |
| A1 | ENSESTT00000049464 | | 8 | 125457337 | 125507878 |
| A1 | ENSESTT00000049463 | | 8 | 125457337 | 125507907 |
| A1 | ENSESTT00000049461 | | 8 | 125457337 | 125507908 |
| A1 | ENSESTT00000049458 | | 8 | 125457337 | 125507913 |
| A1 | ENSESTT00000049467 | | 8 | 125457339 | 125472731 |
| A1 | ENSESTT00000049462 | | 8 | 125457339 | 125507908 |
| A1 | ENST00000276692 | NM_032026 | 8 | 125457339 | 125507917 |
| A1 | ENSESTT00000049465 | | 8 | 125472748 | 125507878 |
| A1 | ENSESTT00000049459 | | 8 | 125472748 | 125507913 |
| A1 | ENSESTT00000049460 | | 8 | 125473099 | 125507913 |
| A1 | ENST00000276689 | NDUFB9 | 8 | 125507932 | 125518808 |
| A1 | ENSESTT00000049446 | | 8 | 125507947 | 125518807 |
| A1 | ENST00000325064 | MTSS1 | 8 | 125519619 | 125697247 |
| A1 | ENSESTT00000049457 | | 8 | 125521907 | 125524630 |
| A1 | ENSESTT00000049456 | | 8 | 125525191 | 125526708 |
| A1 | ENSESTT00000049455 | | 8 | 125526532 | 125537360 |
| A1 | ENSESTT00000049453 | | 8 | 125553967 | 125697188 |
| A1 | ENSESTT00000049454 | | 8 | 125668151 | 125697188 |
| A1 | ENST00000319286 | NM_152412 | 8 | 125942128 | 125948216 |
| A1 | ENSESTT00000049452 | | 8 | 125965815 | 125967028 |
| A1 | ENST00000265896 | SQLE | 8 | 125968234 | 125990776 |
| A1 | ENSESTT00000049447 | | 8 | 125974452 | 125989666 |
| A1 | ENSESTT00000049451 | | 8 | 125987460 | 125991054 |
| A1 | ENSESTT00000049448 | | 8 | 125993091 | 125997546 |
| A1 | ENSESTT00000049450 | | 8 | 125993091 | 126001545 |
| A1 | ENSESTT00000049449 | | 8 | 125993091 | 126006166 |
| A1 | ENST00000318410 | Y196_HUMAN | 8 | 125993448 | 126052729 |
| A1 | ENSESTT00000052951 | | 8 | 126001094 | 126012931 |
| A1 | ENSESTT00000052952 | | 8 | 126001094 | 126012931 |
| A1 | ENSESTT00000052949 | | 8 | 126001094 | 126013531 |
| A1 | ENSESTT00000052944 | | 8 | 126001094 | 126032404 |
| A1 | ENSESTT00000052945 | | 8 | 126001094 | 126032404 |
| A1 | ENSESTT00000052953 | | 8 | 126001148 | 126008731 |
| A1 | ENSESTT00000052950 | | 8 | 126006063 | 126013531 |
| A1 | ENSESTT00000052946 | | 8 | 126006063 | 126032404 |
| A1 | ENSESTT00000052948 | | 8 | 126012674 | 126016164 |
| A1 | ENSESTT00000052947 | | 8 | 126013317 | 126032404 |
| A1 | ENSESTT00000052943 | | 8 | 126036476 | 126043900 |
| A1 | ENSESTT00000052941 | | 8 | 126045186 | 126060614 |
| A1 | ENSESTT00000052942 | | 8 | 126051717 | 126060573 |
| A1 | ENST00000287437 | NM_173685 | 8 | 126060684 | 126335950 |
| A1 | ENSESTT00000052921 | | 8 | 126060694 | 126120112 |
| A1 | ENSESTT00000052935 | | 8 | 126060694 | 126151406 |
| A1 | ENSESTT00000052934 | | 8 | 126060694 | 126326148 |
| A1 | ENSESTT00000052931 | | 8 | 126060694 | 126326652 |
| A1 | ENSESTT00000052932 | | 8 | 126060694 | 126326652 |
| A1 | ENSESTT00000052933 | | 8 | 126060694 | 126326652 |
| A1 | ENSESTT00000052929 | | 8 | 126060694 | 126335951 |
| A1 | ENSESTT00000052930 | | 8 | 126060694 | 126335951 |
| A1 | ENSESTT00000052924 | | 8 | 126060717 | 126151406 |
| A1 | ENSESTT00000052923 | | 8 | 126060717 | 126326652 |
| A1 | ENSESTT00000052922 | | 8 | 126060717 | 126335951 |
| A1 | ENSESTT00000052926 | | 8 | 126060741 | 126151406 |
| A1 | ENSESTT00000052925 | | 8 | 126060741 | 126326652 |
| A1 | ENSESTT00000052927 | | 8 | 126150930 | 126335951 |
| A1 | ENSESTT00000052940 | | 8 | 126317027 | 126320247 |
| A1 | ENSESTT00000052928 | | 8 | 126326536 | 126335951 |
| A1 | ENST00000311922 | NM_025195 | 8 | 126399453 | 126407235 |
| A1 | ENSESTT00000052936 | | 8 | 126399454 | 126404869 |
| A1 | ENSESTT00000052939 | | 8 | 126400000 | 126402312 |
| A1 | ENSESTT00000052937 | | 8 | 126402477 | 126405286 |
| A1 | ENST00000311709 | Q9P1E1 | 8 | 126552574 | 126553590 |
| A1 | ENSESTT00000052938 | | 8 | 126914570 | 126919779 |
| A1 | ENST00000329599 | | 8 | 127041420 | 127043050 |
| A1 | ENSESTT00000046663 | | 8 | 127466983 | 127469137 |
| A1 | ENSESTT00000046664 | | 8 | 127487532 | 127491189 |
| A2 | ENSESTT00000040368 | | 13 | 96395910 | 96638676 |
| A2 | ENSESTT00000040357 | | 13 | 96493435 | 96594999 |
| A2 | OTTHUMT00013002849 | FARP1-006 | 13 | 96493435 | 96595559 |
| A2 | ENSESTT00000040356 | | 13 | 96493435 | 96762294 |
| A2 | OTTHUMT00013002844 | FARP1-001 | 13 | 96493435 | 96800024 |
| A2 | ENST00000319562 | FARP1 | 13 | 96493506 | 96798828 |
| A2 | ENSESTT00000040369 | | 13 | 96526040 | 96526774 |
| A2 | OTTHUMT00013002835 | bA10G5.1-002 | 13 | 96526040 | 96527520 |
| A2 | OTTHUMT00013002834 | bA10G5.1-001 | 13 | 96526425 | 96527547 |
| A2 | ENST00000267291 | ZNF183L1 | 13 | 96526535 | 96527491 |
| A2 | OTTHUMT00013002839 | bA261P24.2-002 | 13 | 96678973 | 96683493 |
| A2 | OTTHUMT00013002838 | bA261P24.2-001 | 13 | 96679135 | 96683493 |
| A2 | ENSESTT00000040358 | | 13 | 96715645 | 96762294 |
| A2 | OTTHUMT00013002845 | FARP1-002 | 13 | 96734616 | 96736041 |
| A2 | OTTHUMT00013002846 | FARP1-003 | 13 | 96754326 | 96762232 |
| A2 | OTTHUMT00013002847 | FARP1-004 | 13 | 96759593 | 96775753 |
| A2 | OTTHUMT00013002848 | FARP1-005 | 13 | 96781485 | 96786357 |
| A2 | OTTHUMT00013002842 | bA111L24.3-001 | 13 | 96785660 | 96786095 |
| A2 | ENSESTT00000040359 | | 13 | 96789051 | 96798697 |
| A2 | OTTHUMT00013002862 | STK24-005 | 13 | 96800456 | 96816816 |
| A2 | OTTHUMT00013002859 | STK24-002 | 13 | 96800456 | 96872252 |
| A2 | OTTHUMT00013002858 | STK24-001 | 13 | 96800456 | 96927118 |
| A2 | ENSESTT00000040367 | | 13 | 96802878 | 96810698 |
| A2 | ENSESTT00000040366 | | 13 | 96802878 | 96816333 |
| A2 | ENSESTT00000040365 | | 13 | 96802878 | 96825181 |
| A2 | ENSESTT00000040363 | | 13 | 96802878 | 96825239 |
| A2 | ENSESTT00000040360 | | 13 | 96802878 | 96927359 |
| A2 | ENSESTT00000040361 | | 13 | 96802878 | 96927359 |
| A2 | ENST00000261573 | STK24 | 13 | 96803428 | 96872107 |
| A2 | OTTHUMT00013002863 | STK24-006 | 13 | 96805676 | 96814040 |
| A2 | OTTHUMT00013002860 | STK24-003 | 13 | 96806237 | 96869522 |
| A2 | ENSESTT00000040364 | | 13 | 96812027 | 96825239 |
| A2 | OTTHUMT00013002861 | STK24-004 | 13 | 96825116 | 96928195 |
| A2 | OTTHUMT00013002864 | STK24-007 | 13 | 96855283 | 96926459 |
| A2 | ENSESTT00000040362 | | 13 | 96855283 | 96926459 |
| A2 | ENST00000313290 | Q8WYY0 | 13 | 96886851 | 96887246 |
| A2 | OTTHUMT00013002856 | bA295B17.5-001 | 13 | 96927499 | 96929085 |
| A2 | OTTHUMT00013002872 | bA295B17.2-001 | 13 | 96960547 | 96961131 |
| A2 | OTTHUMT00013002874 | bA295B17.3-001 | 13 | 96979939 | 96980244 |
| A2 | OTTHUMT00013002876 | bA295B17.4-001 | 13 | 96991069 | 96991494 |
| A2 | OTTHUMT00013002878 | SLC15A1-001 | 13 | 97034056 | 97102908 |
| A2 | ENST00000218552 | SLC15A1 | 13 | 97034979 | 97076822 |
| A2 | OTTHUMT00013002879 | SLC15A1-002 | 13 | 97070782 | 97076822 |
| A2 | ENST00000313260 | O14496 | 13 | 97071712 | 97076756 |
| A2 | OTTHUMT00013002899 | bA155N3.2-010 | 13 | 97143742 | 97182048 |
| A2 | OTTHUMT00013002890 | bA155N3.2-001 | 13 | 97143742 | 97328245 |
| A2 | ENSESTT00000040495 | | 13 | 97144240 | 97147514 |
| A2 | ENSESTT00000040494 | | 13 | 97144240 | 97150734 |
| A2 | ENST00000301980 | DOC9_HUMAN | 13 | 97147367 | 97436606 |
| A2 | OTTHUMT00013002902 | bA155N3.2-013 | 13 | 97150543 | 97179710 |
| A2 | OTTHUMT00013002901 | bA155N3.2-012 | 13 | 97157916 | 97179621 |
| A2 | OTTHUMT00013002898 | bA155N3.2-009 | 13 | 97159564 | 97197939 |
| A2 | ENSESTT00000040492 | | 13 | 97160444 | 97203725 |
| A2 | ENSESTT00000040493 | | 13 | 97160510 | 97182047 |
| A2 | OTTHUMT00013002900 | bA155N3.2-011 | 13 | 97160514 | 97179751 |
| A2 | OTTHUMT00013002896 | bA155N3.2-007 | 13 | 97181651 | 97206239 |
| A2 | OTTHUMT00013002895 | bA155N3.2-006 | 13 | 97182029 | 97200335 |
| A2 | OTTHUMT00013002882 | bA155N3.3-001 | 13 | 97182339 | 97184884 |
| A2 | OTTHUMT00013002894 | bA155N3.2-005 | 13 | 97196203 | 97210731 |
| A2 | ENSESTT00000040491 | | 13 | 97206155 | 97213319 |
| A2 | OTTHUMT00013002897 | bA155N3.2-008 | 13 | 97210436 | 97213803 |
| A2 | ENSESTT00000040490 | | 13 | 97210439 | 97213399 |
| A2 | ENSESTT00000040489 | | 13 | 97217686 | 97230930 |
| A2 | ENSESTT00000040488 | | 13 | 97234078 | 97238454 |
| A2 | OTTHUMT00013002893 | bA155N3.2-004 | 13 | 97238390 | 97238940 |
| A2 | ENSESTT00000040487 | | 13 | 97253255 | 97271344 |
| A2 | ENSESTT00000040486 | | 13 | 97253255 | 97276111 |
| A2 | OTTHUMT00013002891 | bA155N3.2-002 | 13 | 97272306 | 97436880 |
| A2 | ENST00000333692 | | 13 | 97300012 | 97300702 |
| A2 | OTTHUMT00013002884 | bA318G11.2-001 | 13 | 97300012 | 97300702 |
| A2 | OTTHUMT00013002892 | bA155N3.2-003 | 13 | 97305690 | 97436648 |
| A2 | ENSESTT00000040485 | | 13 | 97305748 | 97436584 |
| A2 | OTTHUMT00013002888 | bA122A8.3-001 | 13 | 97523848 | 97524745 |
| A2 | OTTHUMT00013002886 | bA122A8.1-001 | 13 | 97540834 | 97541246 |
| A2 | ENST00000325317 | | 13 | 97541028 | 97541237 |
| A2 | OTTHUMT00013002916 | bA87L10.1-001 | 13 | 97546697 | 97550965 |
| A2 | OTTHUMT00013002917 | bA87L10.1-002 | 13 | 97546697 | 97550965 |
| A2 | ENSESTT00000040484 | | 13 | 97547656 | 97550936 |
| A2 | OTTHUMT00013002939 | bA178C10.1-008 | 13 | 97551029 | 97735570 |
| A2 | OTTHUMT00013002933 | bA178C10.1-002 | 13 | 97551029 | 97735795 |
| A2 | ENSESTT00000040442 | | 13 | 97551031 | 97736683 |
| A2 | ENSESTT00000040438 | | 13 | 97551081 | 97718301 |
| A2 | ENSESTT00000040437 | | 13 | 97551081 | 97736683 |
| A2 | ENSESTT00000040439 | | 13 | 97551109 | 97664477 |
| A2 | OTTHUMT00013002940 | bA178C10.1-009 | 13 | 97551142 | 97690820 |
| A2 | OTTHUMT00013002942 | bA178C10.1-011 | 13 | 97551147 | 97664475 |
| A2 | ENSESTT00000040440 | | 13 | 97551164 | 97718142 |
| A2 | OTTHUMT00013002932 | bA178C10.1-001 | 13 | 97551164 | 97736689 |
| A2 | OTTHUMT00013002941 | bA178C10.1-010 | 13 | 97551171 | 97594879 |
| A2 | ENSESTT00000040441 | | 13 | 97551172 | 97736683 |
| A2 | ENST00000257320 | PHGDHL1 | 13 | 97551667 | 97735590 |
| A2 | OTTHUMT00013002920 | bA461N23.2-001 | 13 | 97565231 | 97566117 |
| A2 | ENST00000325202 | | 13 | 97565627 | 97566013 |
| A2 | ENST00000325028 | PHGDHL1 | 13 | 97588684 | 97735590 |
| A2 | OTTHUMT00013002934 | bA178C10.1-003 | 13 | 97594770 | 97736689 |
| A2 | OTTHUMT00013002928 | GPR18-001 | 13 | 97604987 | 97611999 |
| A2 | ENST00000245300 | GPR18 | 13 | 97604995 | 97608642 |
| A2 | ENSESTT00000040482 | | 13 | 97605550 | 97608614 |
| A2 | OTTHUMT00013002929 | GPR18-002 | 13 | 97605834 | 97608629 |
| A2 | ENSESTT00000040483 | | 13 | 97606624 | 97608610 |
| A2 | OTTHUMT00013002922 | EBI2-001 | 13 | 97644794 | 97657708 |
| A2 | ENST00000301931 | EBI2 | 13 | 97644799 | 97657654 |
| A2 | ENSESTT00000040481 | | 13 | 97645785 | 97657708 |
| A2 | OTTHUMT00013002937 | bA178C10.1-006 | 13 | 97658207 | 97735649 |
| A2 | OTTHUMT00013002935 | bA178C10.1-004 | 13 | 97663038 | 97736689 |
| A2 | OTTHUMT00013002936 | bA178C10.1-005 | 13 | 97664963 | 97735953 |
| A2 | OTTHUMT00013002924 | bA461N23.5-001 | 13 | 97668409 | 97669210 |
| A2 | OTTHUMT00013002926 | bA461N23.6-001 | 13 | 97701675 | 97702282 |
| A2 | OTTHUMT00013002938 | bA178C10.1-007 | 13 | 97718042 | 97727821 |
| A2 | OTTHUMT00013002954 | bA178C10.2-001 | 13 | 97722428 | 97722941 |
| A2 | ENSESTT00000040480 | | 13 | 97736300 | 97736679 |
| A2 | ENSESTT00000040479 | | 13 | 97756206 | 97759085 |
| A2 | OTTHUMT00013002956 | bA178C10.3-001 | 13 | 97758036 | 97759317 |
| A2 | OTTHUMT00013002960 | bA214F16.3-001 | 13 | 97841538 | 97842752 |
| A2 | OTTHUMT00013002958 | bA214F16.2-001 | 13 | 97849982 | 97851307 |
| A2 | ENSESTT00000040461 | | 13 | 97851670 | 97897704 |
| A2 | ENSESTT00000040460 | | 13 | 97851670 | 97905912 |
| A2 | ENSESTT00000040457 | | 13 | 97851670 | 97913646 |
| A2 | ENSESTT00000040458 | | 13 | 97851670 | 97913646 |
| A2 | ENSESTT00000040459 | | 13 | 97851670 | 97913646 |
| A2 | ENSESTT00000040462 | | 13 | 97851727 | 97867945 |
| A2 | OTTHUMT00013002963 | TM9SF2-002 | 13 | 97851729 | 97897328 |
| A2 | OTTHUMT00013002962 | TM9SF2-001 | 13 | 97851729 | 97913644 |
| A2 | ENST00000245361 | TM9SF2 | 13 | 97851862 | 97913013 |
| A2 | OTTHUMT00013002964 | TM9SF2-003 | 13 | 97864470 | 97888117 |
| A2 | ENSESTT00000040467 | | 13 | 97886862 | 97897704 |
| A2 | ENSESTT00000040466 | | 13 | 97886862 | 97905912 |
| A2 | ENSESTT00000040463 | | 13 | 97886862 | 97913646 |
| A2 | ENSESTT00000040464 | | 13 | 97886862 | 97913646 |
| A2 | ENSESTT00000040465 | | 13 | 97886862 | 97913646 |
| A2 | ENSESTT00000040468 | | 13 | 97890373 | 97891991 |
| A2 | OTTHUMT00013002965 | TM9SF2-004 | 13 | 97890766 | 97891991 |
| A2 | ENSESTT00000040471 | | 13 | 97891812 | 97905912 |
| A2 | ENSESTT00000040469 | | 13 | 97891812 | 97913646 |
| A2 | ENSESTT00000040470 | | 13 | 97891812 | 97913646 |
| A2 | ENSESTT00000040473 | | 13 | 97897238 | 97905912 |
| A2 | ENSESTT00000040472 | | 13 | 97897238 | 97909646 |
| A2 | ENSESTT00000040474 | | 13 | 97899412 | 97905912 |
| A2 | ENSESTT00000040476 | | 13 | 97912182 | 97912840 |
| A2 | OTTHUMT00013002970 | bA214F16.4-001 | 13 | 97927367 | 97930623 |
| A2 | OTTHUMT00013002972 | bA214F16.5-001 | 13 | 97932961 | 97933435 |
| A2 | ENSESTT00000040478 | | 13 | 97956924 | 98215198 |
| A2 | ENSESTT00000040477 | | 13 | 97956924 | 98221218 |
| A2 | OTTHUMT00013002980 | CLYBL-001 | 13 | 97956924 | 98242825 |
| A2 | ENST00000323941 | CLYBL | 13 | 97956937 | 98242824 |
| A2 | OTTHUMT00013002981 | CLYBL-002 | 13 | 97956945 | 98241868 |
| A2 | OTTHUMT00013002982 | CLYBL-003 | 13 | 97956951 | 98242819 |
| A2 | OTTHUMT00013002974 | bA279D17.1-001 | 13 | 98040336 | 98041226 |
| A2 | OTTHUMT00013002976 | bA279D17.2-001 | 13 | 98076502 | 98077746 |
| A2 | ENSESTT00000040339 | | 13 | 98076717 | 98079197 |
| A2 | ENSESTT00000040309 | | 13 | 98123162 | 98215493 |
| A2 | OTTHUMT00013002978 | bA134O15.2-001 | 13 | 98124280 | 98125544 |
| A2 | OTTHUMT00013002983 | CLYBL-004 | 13 | 98209116 | 98215493 |
| A2 | OTTHUMT00013002984 | CLYBL-005 | 13 | 98209116 | 98215493 |
| A2 | ENSESTT00000040310 | | 13 | 98213298 | 98242825 |
| A2 | ENSESTT00000040311 | | 13 | 98215060 | 98242825 |
| A2 | ENSESTT00000040312 | | 13 | 98215103 | 98242825 |
| A2 | OTTHUMT00013002986 | CLYBL-007 | 13 | 98216572 | 98243149 |
| A2 | ENSESTT00000040313 | | 13 | 98216572 | 98243149 |
| A2 | OTTHUMT00013002985 | CLYBL-006 | 13 | 98221273 | 98241868 |
| A2 | ENSESTT00000040314 | | 13 | 98235260 | 98241868 |
| A2 | OTTHUMT00013003004 | bA12G12.1-001 | 13 | 98315452 | 98322164 |
| A2 | ENST00000267294 | ZIC5 | 13 | 98315452 | 98322179 |
| A2 | OTTHUMT00013002994 | ZIC2-001 | 13 | 98332294 | 98337019 |
| A2 | ENST00000245295 | ZIC2 | 13 | 98332320 | 98337019 |
| A2 | ENSESTT00000040315 | | 13 | 98332599 | 98335720 |
| A2 | OTTHUMT00013002995 | ZIC2-002 | 13 | 98334544 | 98335747 |
| A2 | ENSESTT00000040316 | | 13 | 98334544 | 98335747 |
| A2 | OTTHUMT00013002997 | ZIC2-004 | 13 | 98334944 | 98335747 |
| A2 | ENSESTT00000040317 | | 13 | 98334944 | 98335747 |
| A2 | OTTHUMT00013002998 | ZIC2-005 | 13 | 98335044 | 98335729 |
| A2 | ENSESTT00000040318 | | 13 | 98335044 | 98335729 |
| A2 | ENSESTT00000040319 | | 13 | 98335196 | 98335747 |
| A2 | OTTHUMT00013002996 | ZIC2-003 | 13 | 98335749 | 98336159 |
| A2 | ENSESTT00000040320 | | 13 | 98335749 | 98336160 |
| A2 | OTTHUMT00013003006 | bA12G12.3-001 | 13 | 98378024 | 98378429 |
| A2 | OTTHUMT00013003008 | bA12G12.4-001 | 13 | 98412551 | 98413856 |
| A2 | ENSESTT00000040321 | | 13 | 98439330 | 98660115 |
| A2 | OTTHUMT00013003012 | PCCA-001 | 13 | 98439338 | 98880687 |
| A2 | ENSESTT00000040322 | | 13 | 98439361 | 98660115 |
| A2 | ENSESTT00000040323 | | 13 | 98439372 | 98660115 |
| A2 | ENSESTT00000040324 | | 13 | 98439395 | 98660115 |
| A2 | ENST00000310787 | PCCA | 13 | 98453138 | 98880421 |
| A2 | OTTHUMT00013003017 | PCCA-006 | 13 | 98499456 | 98511880 |
| A2 | OTTHUMT00013003010 | bA340C20.2-001 | 13 | 98500772 | 98501347 |
| A2 | ENSESTT00000040325 | | 13 | 98585858 | 98660115 |
| A2 | OTTHUMT00013003014 | PCCA-003 | 13 | 98643479 | 98775986 |
| A2 | ENSESTT00000040326 | | 13 | 98643479 | 98881032 |
| A2 | ENSESTT00000040327 | | 13 | 98643479 | 98881032 |
| A2 | ENSESTT00000040328 | | 13 | 98643479 | 98881032 |
| A2 | OTTHUMT00013003013 | PCCA-002 | 13 | 98656031 | 98690514 |
| A2 | ENSESTT00000040329 | | 13 | 98660116 | 98881032 |
| A2 | OTTHUMT00013003015 | PCCA-004 | 13 | 98718727 | 98880687 |
| A2 | OTTHUMT00013003016 | PCCA-005 | 13 | 98718754 | 98842072 |
| A2 | OTTHUMT00013003028 | bA151A6.5-001 | 13 | 98814695 | 98830540 |
| A2 | OTTHUMT00013003030 | bA151A6.5-003 | 13 | 98829802 | 98831270 |
| A2 | OTTHUMT00013003029 | bA151A6.5-002 | 13 | 98829814 | 98831412 |
| A2 | ENSESTT00000040330 | | 13 | 98865680 | 98881032 |
| A2 | OTTHUMT00013003018 | PCCA-007 | 13 | 98865682 | 98880687 |
| A2 | OTTHUMT00013003052 | bA151A6.2-001 | 13 | 98881802 | 98934252 |
| A2 | ENST00000257302 | Q9BT41 | 13 | 98881812 | 98883998 |
| A2 | ENSESTT00000040335 | | 13 | 98882280 | 98934255 |
| A2 | ENSESTT00000040336 | | 13 | 98882742 | 98934255 |
| A2 | ENSESTT00000040338 | | 13 | 98882742 | 98939783 |
| A2 | OTTHUMT00013003055 | bA151A6.2-004 | 13 | 98882750 | 98883924 |
| A2 | OTTHUMT00013003054 | bA151A6.2-003 | 13 | 98882750 | 98939783 |
| A2 | ENSESTT00000040337 | | 13 | 98882766 | 98883908 |
| A2 | OTTHUMT00013003056 | bA151A6.2-005 | 13 | 98882769 | 98883908 |
| A2 | OTTHUMT00013003053 | bA151A6.2-002 | 13 | 98882769 | 98938986 |
| A2 | OTTHUMT00013003034 | bA151A6.4-001 | 13 | 98887578 | 98930372 |
| A2 | OTTHUMT00013003026 | bA151A6.3-001 | 13 | 98890071 | 98890529 |
| A2 | ENST00000245316 | | 13 | 98890156 | 98890503 |
| A2 | OTTHUMT00013003036 | bA113J24.1-001 | 13 | 98954189 | 99025171 |
| A2 | OTTHUMT00013003037 | bA113J24.1-002 | 13 | 98954189 | 99025171 |
| A2 | ENST00000245302 | NM_032813 | 13 | 98955249 | 98989063 |
| A2 | ENSESTT00000040332 | | 13 | 98975664 | 98987865 |
| A2 | ENSESTT00000040334 | | 13 | 98975708 | 98986863 |
| A2 | OTTHUMT00013003038 | bA113J24.1-003 | 13 | 98975708 | 98992566 |
| A2 | OTTHUMT00013003042 | bA113J24.1-007 | 13 | 98975790 | 98986863 |
| A2 | ENSESTT00000040333 | | 13 | 98976035 | 98987865 |
| A2 | OTTHUMT00013003041 | bA113J24.1-006 | 13 | 98985320 | 98986887 |
| A2 | OTTHUMT00013003039 | bA113J24.1-004 | 13 | 98992475 | 99025134 |
| A2 | ENSESTT00000040331 | | 13 | 98992500 | 99014571 |
| A2 | ENSESTT00000040306 | | 13 | 99006632 | 99025080 |
| A2 | ENSESTT00000040307 | | 13 | 99013216 | 99025060 |
| A2 | ENSESTT00000040308 | | 13 | 99018487 | 99020990 |
| A2 | ENSESTT00000040305 | | 13 | 99018833 | 99025134 |
| A2 | OTTHUMT00013003043 | bA113J24.1-008 | 13 | 99018834 | 99025134 |
| A2 | OTTHUMT00013003040 | bA113J24.1-005 | 13 | 99018996 | 99020990 |
| A2 | OTTHUMT00013003066 | bA430M15.1-001 | 13 | 99058580 | 99409639 |
| A2 | OTTHUMT00013003062 | bA190K16.2-001 | 13 | 99106806 | 99107314 |
| A2 | ENST00000310576 | | 13 | 99106812 | 99107311 |
| A2 | ENST00000310558 | Q9BXE6 | 13 | 99111961 | 99112335 |
| A2 | OTTHUMT00013003064 | bA118F16.1-001 | 13 | 99291117 | 99294619 |
| A3 | ENSESTT00000026233 | | 5 | 175066018 | 175091447 |
| A3 | ENST00000231683 | HRH2 | 5 | 175091160 | 175092239 |
| A3 | ENST00000274620 | CPLX2 | 5 | 175204533 | 175288154 |
| A3 | ENST00000274615 | Q96NN7 | 5 | 175288871 | 175289290 |
| A3 | ENST00000334259 | Q8N9L3 | 5 | 175289858 | 175290319 |
| A3 | ENST00000265097 | THOC3 | 5 | 175367459 | 175376203 |
| A3 | ENST00000333723 | | 5 | 175414077 | 175415294 |
| A3 | ENST00000331171 | | 5 | 175414077 | 175415309 |
| A3 | ENST00000330220 | | 5 | 175414083 | 175415294 |
| A3 | ENST00000253490 | Q8TBX6 | 5 | 175492772 | 175517499 |
| A3 | ENSESTT00000026234 | | 5 | 175492798 | 175504815 |
| A3 | ENSESTT00000026235 | | 5 | 175514024 | 175528803 |
| A3 | ENSESTT00000026236 | | 5 | 175531948 | 175533047 |
| A3 | ENSESTT00000026237 | | 5 | 175532319 | 175534252 |
| A3 | ENSESTT00000026238 | | 5 | 175646358 | 175697849 |
| A3 | ENSESTT00000026239 | | 5 | 175646582 | 175697880 |
| A3 | ENST00000303137 | Q8NDZ2 | 5 | 175671357 | 175753911 |
| A3 | ENSESTT00000026240 | | 5 | 175702967 | 175753921 |
| A3 | ENST00000330147 | | 5 | 175714285 | 175714659 |
| A3 | ENST00000332772 | Q8IZ15 | 5 | 175721351 | 175753371 |
| A3 | ENST00000298569 | NM_020444 | 5 | 175753991 | 175769668 |
| A3 | ENSESTT00000026241 | | 5 | 175755867 | 175762014 |
| A3 | ENSESTT00000026242 | | 5 | 175758565 | 175763625 |
| A3 | ENST00000310389 | NM_173664 | 5 | 175773428 | 175781426 |
| A3 | ENSESTT00000026243 | | 5 | 175773498 | 175776896 |
| A3 | ENST00000327101 | CGB7_HUMAN | 5 | 175791865 | 175796511 |
| A3 | ENSESTT00000026244 | | 5 | 175795923 | 175797685 |
| A3 | ENST00000274787 | NM_138820 | 5 | 175796696 | 175797683 |
| A3 | ENSESTT00000026253 | | 5 | 175800471 | 175824467 |
| A3 | ENST00000310407 | CLTB | 5 | 175800698 | 175824287 |
| A3 | ENST00000310418 | NM_001834 | 5 | 175800698 | 175824287 |
| A3 | ENSESTT00000026254 | | 5 | 175800716 | 175824462 |
| A3 | ENSESTT00000026246 | | 5 | 175856279 | 175904920 |
| A3 | ENSESTT00000026245 | | 5 | 175856279 | 175915561 |
| A3 | ENST00000261942 | NM_014613 | 5 | 175856301 | 175917997 |
| A3 | ENSESTT00000026247 | | 5 | 175856353 | 175861483 |
| A3 | ENSESTT00000026248 | | 5 | 175907924 | 175914952 |
| A3 | ENST00000274811 | RNF44 | 5 | 175934638 | 175940396 |
| A3 | ENSESTT00000026249 | | 5 | 175957282 | 175983069 |
| A3 | ENST00000261944 | NM_017675 | 5 | 175957315 | 176003596 |
| A3 | ENSESTT00000026250 | | 5 | 175986469 | 175992466 |
| A3 | ENSESTT00000026251 | | 5 | 175994575 | 176000693 |
| A3 | ENSESTT00000026252 | | 5 | 175994607 | 176000693 |
| A3 | ENST00000303991 | NM_052899 | 5 | 176003728 | 176018054 |
| A3 | ENST00000335532 | Q96PZ4 | 5 | 176004664 | 176007764 |
| A3 | ENST00000310112 | SNCB | 5 | 176028134 | 176037898 |
| A3 | ENST00000318682 | | 5 | 176051522 | 176053904 |
| A3 | ENSESTT00000025931 | | 5 | 176052504 | 176054064 |
| A3 | ENST00000310032 | Q96S98 | 5 | 176055391 | 176066978 |
| A3 | ENSESTT00000025934 | | 5 | 176055433 | 176064052 |
| A3 | ENSESTT00000025933 | | 5 | 176055433 | 176065755 |
| A3 | ENSESTT00000025932 | | 5 | 176055433 | 176065798 |
| A3 | ENST00000274797 | FBXO23 | 5 | 176055504 | 176060711 |
| A3 | ENSESTT00000025937 | | 5 | 176055510 | 176064052 |
| A3 | ENSESTT00000025936 | | 5 | 176055510 | 176065755 |
| A3 | ENSESTT00000025935 | | 5 | 176055510 | 176065798 |
| A3 | ENST00000298564 | Q96FV3 | 5 | 176055540 | 176064848 |
| A3 | ENSESTT00000025938 | | 5 | 176059752 | 176065560 |
| A3 | ENST00000318314 | Q9H7Q1 | 5 | 176062303 | 176065622 |
| A3 | ENST00000329542 | | 5 | 176270544 | 176282324 |
| A3 | ENSESTT00000025945 | | 5 | 176276066 | 176278347 |
| A3 | ENST00000261961 | Q96GP4 | 5 | 176281909 | 176287810 |
| A3 | ENSESTT00000025946 | | 5 | 176281927 | 176285515 |
| A3 | ENST00000292432 | HK3 | 5 | 176288997 | 176304083 |
| A3 | ENST00000323774 | Q9BZR1 | 5 | 176313206 | 176377614 |
| A3 | ENST00000274827 | NM_016290 | 5 | 176313206 | 176390539 |
| A3 | ENSESTT00000025947 | | 5 | 176323181 | 176337765 |
| A3 | ENST00000261948 | NM_012279 | 5 | 176430663 | 176472523 |
| A3 | ENSESTT00000025948 | | 5 | 176430665 | 176472752 |
| A3 | ENSESTT00000025949 | | 5 | 176452251 | 176458816 |
| A3 | ENSESTT00000025951 | | 5 | 176494862 | 176499035 |
| A3 | ENSESTT00000025950 | | 5 | 176494862 | 176500300 |
| A3 | ENSESTT00000025952 | | 5 | 176494947 | 176500300 |
| A3 | ENST00000292408 | FGFR4 | 5 | 176497522 | 176505600 |
| A3 | ENST00000292410 | NM_022963 | 5 | 176497527 | 176505600 |
| A3 | ENSESTT00000025956 | | 5 | 176500568 | 176504578 |
| A3 | ENSESTT00000025957 | | 5 | 176501099 | 176504578 |
| A3 | ENSESTT00000025958 | | 5 | 176505115 | 176506050 |
| A3 | ENSESTT00000025959 | | 5 | 176505286 | 176507234 |
| A3 | ENSESTT00000025960 | | 5 | 176541049 | 176612169 |
| A3 | ENST00000298507 | NSD1 | 5 | 176543028 | 176703698 |
| A3 | ENSESTT00000025961 | | 5 | 176543093 | 176545330 |
| A3 | ENSESTT00000025963 | | 5 | 176612187 | 176617686 |
| A3 | ENSESTT00000025966 | | 5 | 176619693 | 176654715 |
| A3 | ENST00000312855 | | 5 | 176673510 | 176674522 |
| A3 | ENSESTT00000025977 | | 5 | 176699894 | 176702992 |
| A3 | ENSESTT00000025979 | | 5 | 176709391 | 176710012 |
| A3 | ENST00000303270 | RAB24 | 5 | 176709392 | 176711227 |
| A3 | ENSESTT00000025981 | | 5 | 176711722 | 176714120 |
| A3 | ENSESTT00000025980 | | 5 | 176711722 | 176714888 |
| A3 | ENST00000303204 | PX19_HUMAN | 5 | 176711736 | 176714888 |
| A3 | ENST00000303182 | Q96ME3 | 5 | 176715058 | 176719769 |
| A3 | ENST00000303165 | MXD3 | 5 | 176715151 | 176719769 |
| A3 | ENST00000303127 | LMAN2 | 5 | 176739703 | 176759624 |
| A3 | ENSESTT00000025989 | | 5 | 176765891 | 176779165 |
| A3 | ENST00000303066 | RGS14 | 5 | 176765973 | 176780117 |
| A3 | ENSESTT00000025990 | | 5 | 176775288 | 176776866 |
| A3 | ENSESTT00000025991 | | 5 | 176779454 | 176780554 |
| A3 | ENSESTT00000025992 | | 5 | 176792400 | 176794461 |
| A3 | ENST00000324417 | SLC34A1 | 5 | 176792400 | 176806801 |
| A3 | ENSESTT00000025994 | | 5 | 176804912 | 176806144 |
| A3 | ENST00000319628 | | 5 | 176808126 | 176808530 |
| A3 | ENST00000253496 | F12 | 5 | 176810246 | 176817481 |
| A3 | ENSESTT00000025997 | | 5 | 176834655 | 176841379 |
| A3 | ENSESTT00000026002 | | 5 | 176834669 | 176842058 |
| A3 | ENSESTT00000026001 | | 5 | 176834669 | 176844638 |
| A3 | ENSESTT00000025999 | | 5 | 176834669 | 176849007 |
| A3 | ENSESTT00000026000 | | 5 | 176834669 | 176849007 |
| A3 | ENSESTT00000025998 | | 5 | 176834669 | 176850575 |
| A3 | ENST00000230673 | GPRK6 | 5 | 176834797 | 176849743 |
| A3 | ENSESTT00000026007 | | 5 | 176841092 | 176842058 |
| A3 | ENSESTT00000026006 | | 5 | 176841092 | 176844638 |
| A3 | ENSESTT00000026004 | | 5 | 176841092 | 176849007 |
| A3 | ENSESTT00000026005 | | 5 | 176841092 | 176849007 |
| A3 | ENSESTT00000026003 | | 5 | 176841092 | 176850575 |
| A3 | ENSESTT00000025996 | | 5 | 176842854 | 176844638 |
| A3 | ENSESTT00000025995 | | 5 | 176842854 | 176849007 |
| A3 | ENSESTT00000026008 | | 5 | 176842854 | 176850575 |
| A3 | ENST00000323249 | NM_030567 | 5 | 176854746 | 176864135 |
| A3 | ENSESTT00000026009 | | 5 | 176854894 | 176864139 |
| A3 | ENST00000309007 | DBN1 | 5 | 176864466 | 176881467 |
| A3 | ENST00000292385 | NM_080881 | 5 | 176865288 | 176880013 |
| A3 | ENST00000327525 | NM_005451 | 5 | 176891199 | 176905416 |
| A3 | ENST00000328562 | Q14250 | 5 | 176891449 | 176904318 |
| A3 | ENST00000330043 | Q9BXB9 | 5 | 176891449 | 176904318 |
| A3 | ENSESTT00000026018 | | 5 | 176891939 | 177031673 |
| A3 | ENSESTT00000026017 | | 5 | 176897221 | 177038162 |
| A3 | ENSESTT00000026016 | | 5 | 176897716 | 177038203 |
| A3 | ENST00000330641 | Q96C91 | 5 | 176898295 | 176904318 |
| A3 | ENST00000331981 | Q9BXB8 | 5 | 176898906 | 176904318 |
| A3 | ENST00000312943 | Q9BQB3 | 5 | 176909731 | 176916090 |
| A3 | ENST00000274826 | NM_024872 | 5 | 176911807 | 176917676 |
| A3 | ENST00000330503 | DDX41 | 5 | 176919402 | 176924661 |
| A3 | ENSESTT00000026010 | | 5 | 176926890 | 176943796 |
| A3 | ENST00000329540 | NM_019057 | 5 | 176926890 | 176961637 |
| A3 | ENSESTT00000026015 | | 5 | 176945068 | 177094067 |
| A3 | ENSESTT00000026011 | | 5 | 176999291 | 177002623 |
| A3 | ENSESTT00000026013 | | 5 | 176999295 | 177133284 |
| A3 | ENSESTT00000026012 | | 5 | 176999295 | 177136090 |
| A3 | ENST00000328179 | NM_017510 | 5 | 176999300 | 177003200 |
| A3 | ENSESTT00000026014 | | 5 | 176999308 | 177136081 |
| A3 | ENSESTT00000035756 | | 5 | 177000737 | 177003218 |
| A3 | ENSESTT00000035757 | | 5 | 177000783 | 177136346 |
| A3 | ENSESTT00000035758 | | 5 | 177001245 | 177136718 |
| A3 | ENSESTT00000035760 | | 5 | 177007252 | 177150698 |
| A3 | ENST00000333469 | NM_005451 | 5 | 177023996 | 177038192 |
| A3 | ENSESTT00000035801 | | 5 | 177024173 | 177038191 |
| A3 | ENST00000292374 | Q14250 | 5 | 177024246 | 177037114 |
| A3 | ENST00000331561 | Q9BXB9 | 5 | 177024246 | 177037114 |
| A3 | ENST00000331867 | Q96C91 | 5 | 177031099 | 177037114 |
| A3 | ENSESTT00000035800 | | 5 | 177031469 | 177038195 |
| A3 | ENSESTT00000035802 | | 5 | 177031684 | 177037209 |
| A3 | ENST00000332347 | Q9BXB8 | 5 | 177031702 | 177037114 |
| A3 | ENST00000331704 | | 5 | 177042509 | 177048868 |
| A3 | ENST00000333364 | NM_024872 | 5 | 177044585 | 177050454 |
| A3 | ENSESTT00000035798 | | 5 | 177045241 | 177050448 |
| A3 | ENSESTT00000035797 | | 5 | 177045345 | 177050987 |
| A3 | ENSESTT00000035799 | | 5 | 177045385 | 177050435 |
| A3 | ENST00000330228 | DDX41 | 5 | 177052393 | 177057440 |
| A3 | ENSESTT00000035796 | | 5 | 177060391 | 177064902 |
| A3 | ENSESTT00000035761 | | 5 | 177060392 | 177077299 |
| A3 | ENST00000274788 | NM_019057 | 5 | 177060394 | 177072665 |
| A3 | ENSESTT00000035795 | | 5 | 177065352 | 177077000 |
| A3 | ENSESTT00000035794 | | 5 | 177078577 | 177094163 |
| A3 | ENSESTT00000035759 | | 5 | 177132801 | 177136335 |
| A3 | ENST00000332598 | NM_017510 | 5 | 177132810 | 177136711 |
| A3 | ENST00000029410 | B4GALT7 | 5 | 177140813 | 177150931 |
| A3 | ENSESTT00000035762 | | 5 | 177151883 | 177159317 |
| A3 | ENST00000302857 | Q9HAI8 | 5 | 177159504 | 177159962 |
| A3 | ENST00000318185 | | 5 | 177166961 | 177173303 |
| A3 | ENST00000303108 | NM_173663 | 5 | 177263967 | 177321278 |
| A3 | ENST00000324610 | | 5 | 177269572 | 177285570 |
| A3 | ENSESTT00000035793 | | 5 | 177281869 | 177293899 |
| A3 | ENST00000329355 | Q8TE30 | 5 | 177314834 | 177318658 |
| A3 | ENST00000331417 | | 5 | 177377030 | 177378248 |
| A3 | ENST00000328082 | | 5 | 177377030 | 177378266 |
| A3 | ENST00000303154 | THOC3 | 5 | 177416128 | 177424868 |
| A3 | ENSESTT00000035763 | | 5 | 177416183 | 177424870 |
| A3 | ENST00000332215 | Q9H7L9 | 5 | 177511798 | 177512883 |
| A3 | ENST00000308304 | PROP1 | 5 | 177532838 | 177536844 |
| A3 | ENSESTT00000035764 | | 5 | 177582334 | 177589578 |
| A3 | ENSESTT00000035765 | | 5 | 177589720 | 177594471 |
| A3 | ENSESTT00000035766 | | 5 | 177593021 | 177594534 |
| A3 | ENST00000332649 | | 5 | 177596176 | 177596784 |
| A3 | ENST00000274605 | Y341_HUMAN | 5 | 177654157 | 177663002 |
| A3 | ENSESTT00000035767 | | 5 | 177660981 | 177662330 |
| A3 | ENST00000313376 | YE01_HUMAN | 5 | 177671613 | 177689151 |
| A3 | ENSESTT00000035769 | | 5 | 177671744 | 177688239 |
| A3 | ENSESTT00000035768 | | 5 | 177671744 | 177689170 |
| A3 | ENSESTT00000035770 | | 5 | 177684377 | 177689170 |
| A3 | ENSESTT00000035792 | | 5 | 177690065 | 177694571 |
| A3 | ENST00000274606 | NOLA2 | 5 | 177690069 | 177694497 |
| A3 | ENST00000327842 | NM_022471 | 5 | 177726321 | 177727901 |
| A3 | ENST00000261953 | HNRPAB | 5 | 177745109 | 177751782 |
| A3 | ENSESTT00000035781 | | 5 | 177745132 | 177746614 |
| A3 | ENSESTT00000035779 | | 5 | 177745132 | 177751397 |
| A3 | ENSESTT00000035780 | | 5 | 177745132 | 177751397 |
| A3 | ENSESTT00000035777 | | 5 | 177745132 | 177751563 |
| A3 | ENSESTT00000035778 | | 5 | 177745132 | 177751563 |
| A3 | ENSESTT00000035771 | | 5 | 177745132 | 177751762 |
| A3 | ENSESTT00000035772 | | 5 | 177745132 | 177751762 |
| A3 | ENSESTT00000035773 | | 5 | 177745132 | 177751762 |
| A3 | ENSESTT00000035774 | | 5 | 177745132 | 177751762 |
| A3 | ENSESTT00000035775 | | 5 | 177745132 | 177751762 |
| A3 | ENSESTT00000035776 | | 5 | 177745132 | 177751762 |
| A3 | ENST00000307328 | NM_004499 | 5 | 177745440 | 177751225 |
| A3 | ENSESTT00000035782 | | 5 | 177746443 | 177751762 |
| A3 | ENSESTT00000035783 | | 5 | 177747326 | 177751762 |
| A3 | ENSESTT00000035790 | | 5 | 177749103 | 177770674 |
| A3 | ENSESTT00000035787 | | 5 | 177749103 | 177772179 |
| A3 | ENST00000308158 | NM_032921 | 5 | 177749186 | 177773152 |
| A3 | ENSESTT00000035791 | | 5 | 177749189 | 177763185 |
| A3 | ENSESTT00000035788 | | 5 | 177749189 | 177772179 |
| A3 | ENSESTT00000035789 | | 5 | 177749225 | 177772179 |
| A3 | ENSESTT00000035784 | | 5 | 177750732 | 177751762 |
| A3 | ENSESTT00000035786 | | 5 | 177779162 | 177788865 |
| A3 | ENSESTT00000035785 | | 5 | 177891469 | 177892852 |
| A3 | ENSESTT00000025813 | | 5 | 178143370 | 178154143 |
| A3 | ENST00000316308 | CLK4 | 5 | 178144219 | 178171217 |
| A3 | ENSESTT00000025820 | | 5 | 178153016 | 178159432 |
| A3 | ENSESTT00000025818 | | 5 | 178153016 | 178167610 |
| A3 | ENSESTT00000025819 | | 5 | 178157479 | 178160721 |
| A3 | ENSESTT00000025816 | | 5 | 178157835 | 178167668 |
| A3 | ENSESTT00000025814 | | 5 | 178158920 | 178171217 |
| A3 | ENSESTT00000025815 | | 5 | 178159118 | 178167738 |
| A3 | ENSESTT00000025817 | | 5 | 178160838 | 178167632 |
| A3 | ENST00000306591 | ZNF354A | 5 | 178252662 | 178269624 |
| A3 | ENSESTT00000025812 | | 5 | 178267657 | 178269949 |
| A3 | ENSESTT00000025811 | | 5 | 178307485 | 178309124 |
| A3 | ENST00000331699 | | 5 | 178307800 | 178322947 |
| A3 | ENSESTT00000025799 | | 5 | 178400555 | 178423360 |
| A3 | ENST00000322434 | ZNF354B | 5 | 178400555 | 178425630 |
| A3 | ENSESTT00000025800 | | 5 | 178401325 | 178406916 |
| A3 | ENST00000320451 | ZNF271 | 5 | 178472120 | 178473301 |
| A3 | ENST00000320129 | ZNF454 | 5 | 178481825 | 178507035 |
| A3 | ENSESTT00000025801 | | 5 | 178483299 | 178505397 |
| A3 | ENST00000319065 | Q8NHA9 | 5 | 178505697 | 178533033 |
| A3 | ENSESTT00000025802 | | 5 | 178509517 | 178530722 |
| A3 | ENST00000231188 | GRM6 | 5 | 178522259 | 178535546 |
| A3 | ENSESTT00000025810 | | 5 | 178535880 | 178536808 |
| A3 | ENSESTT00000025803 | | 5 | 178564407 | 178573529 |
| A3 | ENST00000315475 | ZNF354C | 5 | 178601202 | 178621290 |
| A3 | ENSESTT00000025809 | | 5 | 178623365 | 178624025 |
| A3 | ENSESTT00000025808 | | 5 | 178653824 | 178654193 |
| A3 | ENSESTT00000025806 | | 5 | 178654498 | 178665387 |
| A3 | ENSESTT00000025807 | | 5 | 178661813 | 178663369 |
| A3 | ENST00000251582 | ADAMTS2 | 5 | 178662264 | 178699481 |
| A3 | ENST00000274609 | ADAMTS2 | 5 | 178691731 | 178885702 |
| A3 | ENSESTT00000025805 | | 5 | 178692740 | 178699439 |
| A3 | ENSESTT00000025804 | | 5 | 178917905 | 178919135 |
| A3 | ENSESTT00000035851 | | 5 | 179043326 | 179055582 |
| A3 | ENSESTT00000035852 | | 5 | 179090766 | 179129747 |
| A3 | ENSESTT00000035853 | | 5 | 179099890 | 179129747 |
| A3 | ENSESTT00000035854 | | 5 | 179099924 | 179129747 |
| A3 | ENST00000319449 | | 5 | 179100164 | 179146212 |
| A3 | ENSESTT00000035855 | | 5 | 179100278 | 179107690 |
| A3 | ENSESTT00000035856 | | 5 | 179107577 | 179131807 |
| A3 | ENSESTT00000035857 | | 5 | 179133252 | 179146705 |
| A3 | ENST00000319571 | NM_030970 | 5 | 179140510 | 179140581 |
| A3 | ENST00000258707 | | 5 | 179148300 | 179148368 |
| A3 | ENSESTT00000035915 | | 5 | 179150871 | 179154755 |
| A3 | ENSESTT00000035916 | | 5 | 179150871 | 179154755 |
| A3 | ENSESTT00000035908 | | 5 | 179150871 | 179160390 |
| A3 | ENSESTT00000035909 | | 5 | 179150871 | 179160390 |
| A3 | ENST00000326748 | HNRPH1 | 5 | 179150872 | 179160359 |
| A3 | ENSESTT00000035918 | | 5 | 179151147 | 179154344 |
| A3 | ENSESTT00000035910 | | 5 | 179151147 | 179160390 |
| A3 | ENSESTT00000035917 | | 5 | 179151282 | 179154755 |
| A3 | ENSESTT00000035911 | | 5 | 179151282 | 179160390 |
| A3 | ENST00000329433 | | 5 | 179152814 | 179157705 |
| A3 | ENSESTT00000035914 | | 5 | 179154867 | 179159858 |
| A3 | ENSESTT00000035912 | | 5 | 179154867 | 179160364 |
| A3 | ENSESTT00000035913 | | 5 | 179157887 | 179160362 |
| A3 | ENST00000331874 | Q8NA96 | 5 | 179176906 | 179177448 |
| A3 | ENSESTT00000035858 | | 5 | 179184188 | 179186866 |
| A3 | ENST00000328856 | Q86VE1 | 5 | 179184188 | 179187649 |
| A3 | ENSESTT00000035859 | | 5 | 179215326 | 179231463 |
| A3 | ENSESTT00000035907 | | 5 | 179215692 | 179217634 |
| A3 | ENST00000329156 | | 5 | 179230857 | 179231354 |
| A3 | ENSESTT00000035861 | | 5 | 179235589 | 179245048 |
| A3 | ENSESTT00000035860 | | 5 | 179235589 | 179267552 |
| A3 | ENST00000247461 | CANX | 5 | 179235655 | 179267584 |
| A3 | ENSESTT00000035865 | | 5 | 179240421 | 179242025 |
| A3 | ENSESTT00000035862 | | 5 | 179243792 | 179267552 |
| A3 | ENSESTT00000035863 | | 5 | 179246533 | 179256391 |
| A3 | ENSESTT00000035864 | | 5 | 179263334 | 179267552 |
| A3 | ENST00000292599 | MAML1 | 5 | 179269772 | 179313948 |
| A3 | ENSESTT00000035866 | | 5 | 179303154 | 179307853 |
| A3 | ENST00000298607 | NM_024978 | 5 | 179314421 | 179314996 |
| A3 | ENST00000292596 | LTC4S | 5 | 179330648 | 179333175 |
| A3 | ENSESTT00000035868 | | 5 | 179330696 | 179333148 |
| A3 | ENSESTT00000035869 | | 5 | 179330774 | 179333175 |
| A3 | ENSESTT00000035870 | | 5 | 179332229 | 179332994 |
| A3 | ENSESTT00000035867 | | 5 | 179332243 | 179333174 |
| A3 | ENST00000292591 | MGAT4B | 5 | 179334262 | 179343246 |
| A3 | ENSESTT00000035906 | | 5 | 179334633 | 179337617 |
| A3 | ENSESTT00000035901 | | 5 | 179334633 | 179338133 |
| A3 | ENSESTT00000035902 | | 5 | 179334633 | 179338133 |
| A3 | ENSESTT00000035895 | | 5 | 179334633 | 179343595 |
| A3 | ENSESTT00000035903 | | 5 | 179334876 | 179338133 |
| A3 | ENSESTT00000035904 | | 5 | 179334883 | 179338133 |
| A3 | ENSESTT00000035900 | | 5 | 179334883 | 179342992 |
| A3 | ENSESTT00000035896 | | 5 | 179334883 | 179343595 |
| A3 | ENSESTT00000035897 | | 5 | 179334883 | 179343595 |
| A3 | ENSESTT00000035905 | | 5 | 179335007 | 179333133 |
| A3 | ENSESTT00000035898 | | 5 | 179335007 | 179343595 |
| A3 | ENSESTT00000035899 | | 5 | 179337988 | 179343595 |
| A3 | ENSESTT00000035871 | | 5 | 179357567 | 179374487 |
| A3 | ENST00000292588 | SQSTM1 | 5 | 179357599 | 179373255 |
| A3 | ENSESTT00000035894 | | 5 | 179370411 | 179373916 |
| A3 | ENST00000292586 | NM_016175 | 5 | 179373936 | 179378682 |
| A3 | ENSESTT00000035893 | | 5 | 179373938 | 179395443 |
| A3 | ENST00000328625 | NM_016175 | 5 | 179374053 | 179395458 |
| A3 | ENST00000261956O75163 | | 5 | 179398735 | 179444516 |
| A3 | ENST00000312107 | NM_015043 | 5 | 179400110 | 179407115 |
| A3 | ENSESTT00000035892 | | 5 | 179400203 | 179402065 |
| A3 | ENSESTT00000035891 | | 5 | 179400212 | 179402552 |
| A3 | ENSESTT00000035890 | | 5 | 179400359 | 179404512 |
| A3 | ENSESTT00000035889 | | 5 | 179409551 | 179415194 |
| A3 | ENSESTT00000035887 | | 5 | 179415747 | 179444521 |
| A3 | ENSESTT00000035888 | | 5 | 179416330 | 179444521 |
| A3 | ENSESTT00000035886 | | 5 | 179492143 | 179507146 |
| A3 | ENSESTT00000035885 | | 5 | 179492144 | 179514948 |
| A3 | ENSESTT00000035884 | | 5 | 179492144 | 179549976 |
| A3 | ENSESTT00000035881 | | 5 | 179492144 | 179608429 |
| A3 | ENST00000261947 | RNF130 | 5 | 179492168 | 179608763 |
| A3 | ENSESTT00000035882 | | 5 | 179514859 | 179608429 |
| A3 | ENSESTT00000035883 | | 5 | 179516790 | 179608429 |
| A3 | ENST00000332144 | | 5 | 179622379 | 179623665 |
| A3 | ENST00000274820 | NM_175062 | 5 | 179638729 | 179745830 |
| A3 | ENST00000316131 | | 5 | 179763049 | 179763664 |
| A3 | ENSESTT00000035880 | | 5 | 179772812 | 179779418 |
| A3 | ENSESTT00000035877 | | 5 | 179772812 | 179817172 |
| A3 | ENST00000057533 | NM_139069 | 5 | 179773046 | 179817223 |
| A3 | ENST00000316123 | MAPK9 | 5 | 179773046 | 179817223 |
| A3 | ENSESTT00000035879 | | 5 | 179783749 | 179798444 |
| A3 | ENSESTT00000035878 | | 5 | 179790169 | 179801503 |
| A3 | ENST00000328081 | | 5 | 179794621 | 179796637 |
| A3 | ENSESTT00000035876 | | 5 | 179816486 | 179828537 |
| A3 | ENST00000253778 | GFPT2 | 5 | 179837361 | 179875263 |
| A3 | ENSESTT00000035874 | | 5 | 179837891 | 179868310 |
| A3 | ENSESTT00000035875 | | 5 | 179861517 | 179867483 |
| A3 | ENSESTT00000035873 | | 5 | 179868152 | 179889985 |
| A3 | ENSESTT00000035872 | | 5 | 179872531 | 179889987 |
| A3 | ENST00000261951 | NM_015455 | 5 | 180065924 | 180110862 |
| A3 | ENSESTT00000035640 | | 5 | 180065968 | 180087268 |
| A3 | ENSESTT00000035639 | | 5 | 180065968 | 180101184 |
| A3 | ENST00000332929 | Q8TAJ0 | 5 | 180112873 | 180113151 |
| A3 | ENSESTT00000035680 | | 5 | 180126767 | 180128147 |
| A3 | ENST00000292641 | SCGB3A1 | 5 | 180126768 | 180128145 |
| A3 | ENSESTT00000035679 | | 5 | 180139821 | 180148082 |
| A3 | ENST00000261937 | FLT4 | 5 | 180145626 | 180186207 |
| A3 | ENSESTT00000035678 | | 5 | 180162667 | 180166645 |
| A3 | ENSESTT00000035677 | | 5 | 180166890 | 180186286 |
| A3 | ENST00000315712 | Q8NHB0 | 5 | 180229572 | 180230422 |
| A3 | ENST00000307832 | Q8NGV0 | 5 | 180275785 | 180276720 |
| A3 | ENSESTT00000035676 | | 5 | 180327203 | 180328628 |
| A3 | ENSESTT00000035675 | | 5 | 180327203 | 180328768 |
| A3 | ENST00000333055 | MGAT1 | 5 | 180327210 | 180352203 |
| A3 | ENSESTT00000035663 | | 5 | 180327543 | 180339485 |
| A3 | ENST00000307826 | Q8NBL8 | 5 | 180328296 | 180329633 |
| A3 | ENSESTT00000035662 | | 5 | 180329112 | 180339503 |
| A3 | ENSESTT00000035659 | | 5 | 180329155 | 180345445 |
| A3 | ENSESTT00000035661 | | 5 | 180329332 | 180340541 |
| A3 | ENSESTT00000035665 | | 5 | 180332068 | 180352229 |
| A3 | ENSESTT00000035664 | | 5 | 180332092 | 180339429 |
| A3 | ENSESTT00000035660 | | 5 | 180332309 | 180345357 |
| A3 | ENSESTT00000035658 | | 5 | 180344545 | 180346738 |
| A3 | ENSESTT00000035666 | | 5 | 180345508 | 180352200 |
| A3 | ENSESTT00000035657 | | 5 | 180345511 | 180346792 |
| A3 | ENSESTT00000035673 | | 5 | 180384273 | 180397228 |
| A3 | ENSESTT00000035672 | | 5 | 180384335 | 180397947 |
| A3 | ENST00000330037 | NM_152283 | 5 | 180385559 | 180387766 |
| A3 | ENST00000302108 | | 5 | 180386417 | 180388132 |
| A3 | ENSESTT00000035674 | | 5 | 180387577 | 180397213 |
| A3 | ENST00000231229 | NM_024850 | 5 | 180435821 | 180487566 |
| A3 | ENSESTT00000035641 | | 5 | 180435955 | 180448194 |
| A3 | ENSESTT00000035642 | | 5 | 180448211 | 180487568 |
| A3 | ENST00000301996 | BTNL3 | 5 | 180525529 | 180543025 |
| A3 | ENST00000298708 | NM_152547 | 5 | 180582152 | 180590225 |
| A3 | ENSESTT00000035643 | | 5 | 180589782 | 180591964 |
| A3 | ENST00000327705 | Q8N324 | 5 | 180590141 | 180598180 |
| A3 | ENSESTT00000035644 | | 5 | 180592305 | 180595974 |
| A3 | ENST00000328095 | | 5 | 180635802 | 180637319 |
| A3 | ENST00000328767 | | 5 | 180650805 | 180651920 |
| A3 | ENST00000329365 | | 5 | 180661176 | 180661966 |
| A3 | ENST00000328275 | Q8NGV1 | 5 | 180691605 | 180692552 |
| A3 | ENSESTT00000035645 | | 5 | 180728586 | 180731091 |
| A3 | ENST00000274773 | Q96J89 | 5 | 180731275 | 180736815 |
| A3 | ENSESTT00000035646 | | 5 | 180734864 | 180739766 |
| A3 | ENSESTT00000035671 | | 5 | 180739916 | 180741785 |
| A3 | ENST00000334421 | TRIM7 | 5 | 180740159 | 180741772 |
| A3 | ENST00000312487 | Q96Q10 | 5 | 180760899 | 180772041 |
| A3 | ENST00000315073 | TRIM41 | 5 | 180760917 | 180772467 |
| A3 | ENSESTT00000035647 | | 5 | 180769651 | 180772470 |
| A3 | ENSESTT00000035648 | | 5 | 180770071 | 180772470 |
| A3 | ENST00000274821 | GNB2L1 | 5 | 180773587 | 180780586 |
| A3 | ENSESTT00000035670 | | 5 | 180773591 | 180776245 |
| A3 | ENSESTT00000035649 | | 5 | 180783187 | 180783850 |
| A3 | ENSESTT00000035669 | | 5 | 180791085 | 180796857 |
| A3 | ENST00000327725 | NM_022907 | 5 | 180792443 | 180792880 |
| A3 | ENSESTT00000035668 | | 5 | 180793051 | 180796920 |
| A3 | ENSESTT00000035667 | | 5 | 180793672 | 180796987 |
| A3 | ENST00000327767 | TRIM52 | 5 | 180794077 | 180797476 |
| A3 | ENSESTT00000035652 | | 5 | 180797875 | 180800890 |
| A3 | ENSESTT00000035651 | | 5 | 180797875 | 180800918 |
| A3 | ENSESTT00000035650 | | 5 | 180797875 | 180800920 |
| A3 | ENSESTT00000035653 | | 5 | 180854088 | 180864732 |
| A3 | ENSESTT00000035654 | | 5 | 180866068 | 180886905 |
| A3 | ENST00000333864 | O4F3_HUMAN | 5 | 180903950 | 180904888 |
| A3 | ENSESTT00000035655 | | 5 | 180988607 | 180991662 |
| A3 | ENSESTT00000035656 | | 5 | 180990904 | 180991624 |
| A3 | ENST00000332522 | | 5 | 181008629 | 181009085 |
| A4 | OTTHUMT00013000706 | FLT1-001 | 13 | 26672489 | 26867232 |
| A4 | ENST00000282397 | FLT1 | 13 | 26675304 | 26867254 |
| A4 | ENSESTT00000037419 | | 13 | 26740528 | 26762136 |
| A4 | ENSESTT00000037420 | | 13 | 26749569 | 26762136 |
| A4 | ENSESTT00000037421 | | 13 | 26756741 | 26762136 |
| A4 | ENSESTT00000037417 | | 13 | 26769173 | 26810483 |
| A4 | ENSESTT00000037415 | | 13 | 26769173 | 26839090 |
| A4 | ENSESTT00000037418 | | 13 | 26772654 | 26810483 |
| A4 | ENSESTT00000037416 | | 13 | 26772654 | 26839090 |
| A4 | ENSESTT00000037414 | | 13 | 26839038 | 26867241 |
| A4 | OTTHUMT00013000708 | bA57H24.1-001 | 13 | 26970970 | 26972189 |
| A4 | ENST00000255315 | C13orf12 | 13 | 27031241 | 27051059 |
| A4 | OTTHUMT00013000710 | bA97E23.1-001 | 13 | 27031241 | 27051062 |
| A4 | OTTHUMT00013000711 | bA97E23.1-002 | 13 | 27031241 | 27051062 |
| A4 | ENSESTT00000037329 | | 13 | 27031251 | 27051047 |
| A4 | ENSESTT00000037330 | | 13 | 27031270 | 27034993 |
| A4 | ENSESTT00000037328 | | 13 | 27032876 | 27050475 |
| A4 | ENSESTT00000037342 | | 13 | 27044487 | 27050337 |
| A4 | OTTHUMT00013000715 | bA97E23.2-002 | 13 | 27072201 | 27076694 |
| A4 | ENST00000266943 | NM_181785 | 13 | 27072854 | 27090734 |
| A4 | OTTHUMT00013000714 | bA97E23.2-001 | 13 | 27072854 | 27091107 |
| A4 | ENSESTT00000037341 | | 13 | 27073385 | 27082982 |
| A4 | ENSESTT00000037340 | | 13 | 27073407 | 27085276 |
| A4 | ENSESTT00000037339 | | 13 | 27085474 | 27091111 |
| A4 | OTTHUMT00013000718 | bA97E23.3-001 | 13 | 27094523 | 27096158 |
| A4 | ENSESTT00000037338 | | 13 | 27094840 | 27095270 |
| A4 | OTTHUMT00013000720 | bA161P17.1-001 | 13 | 27150695 | 27151074 |
| A4 | ENST00000255289 | Q8N5E2 | 13 | 27397451 | 27875542 |
| A4 | OTTHUMT00013000739 | bA274A8.1-002 | 13 | 27397451 | 27875892 |
| A4 | OTTHUMT00013000726 | bA351N4.3-003 | 13 | 27611516 | 27622691 |
| A4 | OTTHUMT00013000725 | bA351N4.3-002 | 13 | 27611760 | 27622691 |
| A4 | OTTHUMT00013000724 | bA351N4.3-001 | 13 | 27611924 | 27622691 |
| A4 | ENSESTT00000037331 | | 13 | 27653849 | 27673230 |
| A4 | OTTHUMT00013000722 | bA351N4.2-001 | 13 | 27678843 | 27680052 |
| A4 | OTTHUMT00013000738 | bA274A8.1-001 | 13 | 27800764 | 27875873 |
| A4 | ENSESTT00000037332 | | 13 | 27800922 | 27869445 |
| A4 | ENST00000323380 | Q8N642 | 13 | 27848687 | 27859887 |
| A4 | OTTHUMT00013000730 | bA274A8.2-001 | 13 | 27849033 | 27859887 |
| A4 | ENSESTT00000037337 | | 13 | 27849033 | 27859887 |
| A4 | OTTHUMT00013000732 | SLC7A1-001 | 13 | 27881547 | 27967721 |
| A4 | ENST00000266949 | SLC7A1 | 13 | 27886617 | 27908325 |
| A4 | OTTHUMT00013000733 | SLC7A1-002 | 13 | 27889004 | 27891647 |
| A4 | ENSESTT00000037336 | | 13 | 27889008 | 27894543 |
| A4 | ENSESTT00000037335 | | 13 | 27894554 | 27902860 |
| A4 | ENSESTT00000037333 | | 13 | 27907953 | 27967721 |
| A4 | ENSESTT00000037334 | | 13 | 27907955 | 27958929 |
| A4 | OTTHUMT00013000734 | SLC7A1-003 | 13 | 27907961 | 27958929 |
| A4 | ENST00000330321 | Q8NI69 | 13 | 27964863 | 27965033 |
| A4 | ENST00000255303 | Q8TE30 | 13 | 28014062 | 28017889 |
| A4 | ENST00000310635 | Q9P1E1 | 13 | 28017953 | 28018969 |
| A4 | OTTHUMT00013000746 | UBL3-001 | 13 | 28136508 | 28222821 |
| A4 | ENST00000241470 | | 13 | 28136546 | 28222715 |
| A4 | ENSESTT00000037443 | | 13 | 28138914 | 28222162 |
| A4 | ENSESTT00000037445 | | 13 | 28139131 | 28144336 |
| A4 | ENSESTT00000037444 | | 13 | 28139753 | 28222162 |
| A4 | OTTHUMT00013000744 | bA90M5.2-001 | 13 | 28238096 | 28238306 |
| A4 | OTTHUMT00013000748 | bA90M5.4-001 | 13 | 28290784 | 28298788 |
| A4 | ENSESTT00000037431 | | 13 | 28310040 | 28322630 |
| A4 | ENST00000241471 | Q9H523 | 13 | 28310043 | 28322495 |
| A4 | OTTHUMT00013000742 | bA90M5.1-001 | 13 | 28310043 | 28322628 |
| A4 | OTTHUMT00013000750 | bA629E24.1-001 | 13 | 28479753 | 28481012 |
| A4 | OTTHUMT00013000752 | bA490N5.1-001 | 13 | 28526203 | 28526723 |
| A4 | OTTHUMT00013000754 | bA374F3.1-001 | 13 | 28580177 | 28679585 |
| A4 | ENSESTT00000037442 | | 13 | 28580405 | 28603509 |
| A4 | ENST00000261628 | NM_032116 | 13 | 28580617 | 28679163 |
| A4 | ENSESTT00000037439 | | 13 | 28627583 | 28655933 |
| A4 | ENSESTT00000037440 | | 13 | 28627583 | 28679620 |
| A4 | ENSESTT00000037438 | | 13 | 28627618 | 28679146 |
| A4 | ENSESTT00000037441 | | 13 | 28627625 | 28679181 |
| A4 | OTTHUMT00013000756 | bA374F3.2-001 | 13 | 28668272 | 28668860 |
| A4 | ENST00000319015 | | 13 | 28668284 | 28668872 |
| A4 | OTTHUMT00013000758 | bA374F3.3-001 | 13 | 28688497 | 28692033 |
| A4 | OTTHUMT00013000761 | bA374F3.4-002 | 13 | 28713496 | 28736232 |
| A4 | ENSESTT00000037437 | | 13 | 28713496 | 28736232 |
| A4 | OTTHUMT00013000760 | bA374F3.4-001 | 13 | 28714620 | 28737898 |
| A4 | OTTHUMT00013000763 | bA374F3.4-004 | 13 | 28729879 | 28736287 |
| A4 | OTTHUMT00013000762 | bA374F3.4-003 | 13 | 28729879 | 28749282 |
| A4 | OTTHUMT00013000764 | bA374F3.4-005 | 13 | 28735317 | 28745169 |
| A4 | OTTHUMT00013000765 | bA374F3.4-006 | 13 | 28746140 | 28749101 |
| A4 | OTTHUMT00013000772 | bA223E19.1-001 | 13 | 28800091 | 28800795 |
| A4 | ENST00000302464 | UBE2L3 | 13 | 28800128 | 28800592 |
| A4 | OTTHUMT00013000792 | bA550P23.2-001 | 13 | 28818545 | 28820625 |
| A4 | OTTHUMT00013000777 | HMGB1-004 | 13 | 28831990 | 28838109 |
| A4 | ENST00000255320 | HMGB1 | 13 | 28831997 | 28838013 |
| A4 | ENSESTT00000037436 | | 13 | 28833364 | 28838063 |
| A4 | OTTHUMT00013000774 | HMGB1-001 | 13 | 28833566 | 28836447 |
| A4 | OTTHUMT00013000775 | HMGB1-002 | 13 | 28833621 | 28836447 |
| A4 | OTTHUMT00013000776 | HMGB1-003 | 13 | 28833633 | 28838075 |
| A4 | ENSESTT00000037435 | | 13 | 28834423 | 28989492 |
| A4 | OTTHUMT00013000781 | HMGB1-008 | 13 | 28834483 | 28835401 |
| A4 | OTTHUMT00013000782 | HMGB1-009 | 13 | 28834675 | 28836526 |
| A4 | ENSESTT00000037434 | | 13 | 28834713 | 28989872 |
| A4 | OTTHUMT00013000779 | HMGB1-006 | 13 | 28834750 | 28836793 |
| A4 | OTTHUMT00013000778 | HMGB1-005 | 13 | 28835348 | 28838651 |
| A4 | OTTHUMT00013000780 | HMGB1-007 | 13 | 28835448 | 28838051 |
| A4 | OTTHUMT00013000794 | bA550P23.3-001 | 13 | 28911734 | 28915598 |
| A4 | OTTHUMT00013000796 | bA550P23.4-001 | 13 | 28913780 | 28915034 |
| A4 | OTTHUMT00013000798 | bA550P23.5-001 | 13 | 28925807 | 28926830 |
| A4 | OTTHUMT00013000801 | bA121O19.1-002 | 13 | 28989830 | 29003611 |
| A4 | ENST00000255304 | NM_005800 | 13 | 28989920 | 29031493 |
| A4 | OTTHUMT00013000800 | bA121O19.1-001 | 13 | 28989920 | 29031686 |
| A4 | ENSESTT00000037432 | | 13 | 28990053 | 29003351 |
| A4 | ENSESTT00000037433 | | 13 | 28990087 | 29003261 |
| A4 | ENSESTT00000042385 | | 13 | 29014843 | 29031687 |
| A4 | OTTHUMT00013000806 | ALOX5AP-001 | 13 | 29107645 | 29136556 |
| A4 | ENST00000255317 | ALOX5AP | 13 | 29107669 | 29136562 |
| A4 | OTTHUMT00013000807 | ALOX5AP-002 | 13 | 29114473 | 29116533 |
| A4 | OTTHUMT00013000804 | bA469L23.2-001 | 13 | 29175343 | 29182782 |
| A4 | ENSESTT00000042392 | | 13 | 29175343 | 29183024 |
| A4 | ENSESTT00000042393 | | 13 | 29202772 | 29255532 |
| A4 | OTTHUMT00013000810 | bA252M21.1-001 | 13 | 29254688 | 29255532 |
| A4 | ENSESTT00000042394 | | 13 | 29254696 | 29255532 |
| A4 | ENST00000218987 | NM_032849 | 13 | 29278328 | 29297708 |
| A4 | OTTHUMT00013000812 | bA252M21.2-001 | 13 | 29278328 | 29297709 |
| A4 | OTTHUMT00013000813 | bA252M21.2-002 | 13 | 29278843 | 29296586 |
| A4 | ENSESTT00000042395 | | 13 | 29289536 | 29297968 |
| A4 | ENSESTT00000042471 | | 13 | 29302805 | 29303625 |
| A4 | OTTHUMT00013000816 | bA252M21.3-001 | 13 | 29302814 | 29303619 |
| A4 | OTTHUMT00013000818 | bA252M21.4-001 | 13 | 29303638 | 29304723 |
| A4 | OTTHUMT00013000819 | bA252M21.4-002 | 13 | 29303766 | 29304337 |
| A4 | OTTHUMT00013000823 | bA252M21.5-002 | 13 | 29304840 | 29305327 |
| A4 | OTTHUMT00013000822 | bA252M21.5-001 | 13 | 29304840 | 29347639 |
| A4 | ENST00000320096 | NM_152325 | 13 | 29304853 | 29347044 |
| A4 | ENSESTT00000042396 | | 13 | 29329009 | 29347274 |
| A4 | OTTHUMT00013000826 | bA252M21.6-001 | 13 | 29345426 | 29349760 |
| A4 | OTTHUMT00013000828 | bA252M21.7-001 | 13 | 29367299 | 29368275 |
| A4 | OTTHUMT00013000830 | bA173P16.1-001 | 13 | 29471238 | 29487836 |
| A4 | ENSESTT00000042440 | | 13 | 29508750 | 29511044 |
| A4 | ENSESTT00000042437 | | 13 | 29508750 | 29511319 |
| A4 | ENSESTT00000042416 | | 13 | 29508750 | 29520230 |
| A4 | ENSESTT00000042418 | | 13 | 29508750 | 29520230 |
| A4 | ENSESTT00000042420 | | 13 | 29508750 | 29520230 |
| A4 | ENSESTT00000042421 | | 13 | 29508750 | 29520230 |
| A4 | ENSESTT00000042403 | | 13 | 29508750 | 29520554 |
| A4 | ENSESTT00000042405 | | 13 | 29508750 | 29520554 |
| A4 | ENSESTT00000042406 | | 13 | 29508750 | 29520554 |
| A4 | ENSESTT00000042407 | | 13 | 29508750 | 29520554 |
| A4 | ENSESTT00000042438 | | 13 | 29508750 | 29520623 |
| A4 | ENSESTT00000042443 | | 13 | 29508750 | 29520623 |
| A4 | ENSESTT00000042444 | | 13 | 29508750 | 29520623 |
| A4 | ENSESTT00000042445 | | 13 | 29508750 | 29520623 |
| A4 | ENSESTT00000042402 | | 13 | 29508750 | 29523880 |
| A4 | ENSESTT00000042404 | | 13 | 29508750 | 29523880 |
| A4 | ENSESTT00000042413 | | 13 | 29508750 | 29523880 |
| A4 | ENSESTT00000042414 | | 13 | 29508750 | 29523880 |
| A4 | ENSESTT00000042442 | | 13 | 29508762 | 29509663 |
| A4 | OTTHUMT00013000835 | bA173P16.2-004 | 13 | 29508762 | 29510705 |
| A4 | OTTHUMT00013000832 | bA173P16.2-001 | 13 | 29508765 | 29534064 |
| A4 | OTTHUMT00013000833 | bA173P16.2-002 | 13 | 29508765 | 29534064 |
| A4 | OTTHUMT00013000834 | bA173P16.2-003 | 13 | 29508765 | 29534064 |
| A4 | ENSESTT00000042441 | | 13 | 29509002 | 29511044 |
| A4 | ENSESTT00000042422 | | 13 | 29509002 | 29520230 |
| A4 | ENSESTT00000042423 | | 13 | 29509002 | 29520230 |
| A4 | ENSESTT00000042408 | | 13 | 29509002 | 29520554 |
| A4 | ENSESTT00000042409 | | 13 | 29509002 | 29520554 |
| A4 | ENSESTT00000042446 | | 13 | 29509002 | 29520623 |
| A4 | ENSESTT00000042447 | | 13 | 29509002 | 29520623 |
| A4 | ENSESTT00000042415 | | 13 | 29509002 | 29523880 |
| A4 | ENSESTT00000042417 | | 13 | 29509002 | 29523880 |
| A4 | ENST00000239887 | HSPH1 | 13 | 29509455 | 29533719 |
| A4 | ENST00000320027 | HSPH1 | 13 | 29509455 | 29533719 |
| A4 | ENSESTT00000042439 | | 13 | 29509641 | 29511319 |
| A4 | ENSESTT00000042424 | | 13 | 29510418 | 29520230 |
| A4 | ENSESTT00000042410 | | 13 | 29510418 | 29520554 |
| A4 | ENSESTT00000042448 | | 13 | 29510418 | 29520623 |
| A4 | ENSESTT00000042419 | | 13 | 29510418 | 29523880 |
| A4 | ENSESTT00000042425 | | 13 | 29510574 | 29520230 |
| A4 | ENSESTT00000042432 | | 13 | 29510574 | 29522321 |
| A4 | ENSESTT00000042428 | | 13 | 29510574 | 29523880 |
| A4 | OTTHUMT00013000836 | bA173P16.2-005 | 13 | 29510676 | 29511319 |
| A4 | ENSESTT00000042426 | | 13 | 29510913 | 29520230 |
| A4 | ENSESTT00000042427 | | 13 | 29510913 | 29520230 |
| A4 | ENSESTT00000042411 | | 13 | 29510913 | 29520554 |
| A4 | ENSESTT00000042449 | | 13 | 29510913 | 29520623 |
| A4 | ENSESTT00000042433 | | 13 | 29510913 | 29522321 |
| A4 | ENSESTT00000042429 | | 13 | 29510913 | 29523880 |
| A4 | ENSESTT00000042430 | | 13 | 29510913 | 29523880 |
| A4 | ENSESTT00000042436 | | 13 | 29515928 | 29520230 |
| A4 | ENSESTT00000042412 | | 13 | 29515928 | 29520554 |
| A4 | ENSESTT00000042401 | | 13 | 29515928 | 29520623 |
| A4 | ENSESTT00000042434 | | 13 | 29515928 | 29522321 |
| A4 | ENSESTT00000042431 | | 13 | 29515928 | 29523880 |
| A4 | ENSESTT00000042435 | | 13 | 29520095 | 29522321 |
| A4 | ENSESTT00000042400 | | 13 | 29527641 | 29534068 |
| A4 | ENSESTT00000042399 | | 13 | 29527667 | 29534489 |
| A4 | ENSESTT00000042397 | | 13 | 29572112 | 29633161 |
| A4 | OTTHUMT00013000842 | bA367C11.1-001 | 13 | 29572112 | 29704409 |
| A4 | ENSESTT00000042398 | | 13 | 29632535 | 29656892 |
| A4 | OTTHUMT00013000843 | bA367C11.1-002 | 13 | 29632544 | 29656892 |
| A4 | ENST00000310319 | Q9P1E1 | 13 | 29675357 | 29676373 |
| A4 | OTTHUMT00013000846 | bA367C11.2-001 | 13 | 29683426 | 29685058 |
| A4 | ENST00000298386 | LGR8_HUMAN | 13 | 30111674 | 30175009 |
| A4 | OTTHUMT00013000848 | bA432E15.1-001 | 13 | 30111679 | 30175009 |
| A4 | ENSESTT00000037457 | | 13 | 30218905 | 30325581 |
| A4 | ENSESTT00000037458 | | 13 | 30218972 | 30324856 |
| A4 | OTTHUMT00013000850 | Em:AC002525.1-001 | 13 | 30324632 | 30325609 |
| A4 | ENST00000306722 | | 13 | 30324717 | 30325498 |
| A4 | OTTHUMT00013000852 | bA207N4.2-001 | 13 | 30397451 | 30403776 |
| A4 | OTTHUMT00013000854 | bA37E23.1-001 | 13 | 30403437 | 30668794 |
| A4 | ENSESTT00000037459 | | 13 | 30403557 | 30451064 |
| A4 | OTTHUMT00013000855 | bA37E23.1-002 | 13 | 30403692 | 30410757 |
| A4 | ENSESTT00000037460 | | 13 | 30403692 | 30410757 |
| A4 | ENST00000318671 | Q9H551 | 13 | 30403933 | 30474153 |
| A4 | OTTHUMT00013000856 | bA37E23.1-003 | 13 | 30433001 | 30489607 |
| A4 | ENST00000267067 | Q99993 | 13 | 30457981 | 30529546 |
| A4 | ENSESTT00000037461 | | 13 | 30533334 | 30543341 |
| A4 | ENSESTT00000037462 | | 13 | 30545609 | 30551036 |
| A4 | ENST00000261575 | NM_023037 | 13 | 30574067 | 30667597 |
| A4 | ENSESTT00000037446 | | 13 | 30603413 | 30668738 |
| A4 | ENSESTT00000037447 | | 13 | 30606797 | 30609886 |
| A4 | OTTHUMT00013000858 | bA37E23.1-005 | 13 | 30621676 | 30650976 |
| A4 | ENSESTT00000037448 | | 13 | 30621802 | 30637786 |
| A4 | ENSESTT00000037449 | | 13 | 30626285 | 30634614 |
| A4 | ENSESTT00000037450 | | 13 | 30626290 | 30637786 |
| A4 | ENSESTT00000037453 | | 13 | 30626512 | 30650976 |
| A4 | ENSESTT00000037454 | | 13 | 30626512 | 30650976 |
| A4 | ENSESTT00000037452 | | 13 | 30626512 | 30661841 |
| A4 | ENSESTT00000037451 | | 13 | 30626512 | 30661882 |
| A4 | OTTHUMT00013000859 | bA37E23.1-006 | 13 | 30634526 | 30661776 |
| A4 | ENSESTT00000037455 | | 13 | 30659981 | 30668767 |
| A4 | OTTHUMT00013000857 | bA37E23.1-004 | 13 | 30659988 | 30667640 |
| A4 | ENSESTT00000037456 | | 13 | 30666374 | 30668767 |
| A4 | OTTHUMT00013000870 | bA37E23.5-001 | 13 | 30670291 | 30670849 |
| A4 | OTTHUMT00013000868 | bA37E23.4-001 | 13 | 30680855 | 30683420 |
| A4 | ENSESTT00000037463 | | 13 | 30687607 | 30698292 |
| A4 | OTTHUMT00013003674 | BRCA2-001 | 13 | 30687617 | 30771806 |
| A4 | ENSESTT00000037464 | | 13 | 30687640 | 30698728 |
| A4 | ENST00000267071 | BRCA2 | 13 | 30688598 | 30770907 |
| A4 | ENSESTT00000037465 | | 13 | 30735410 | 30752226 |
| A4 | ENSESTT00000037466 | | 13 | 30748806 | 30752226 |
| A4 | OTTHUMT00013003675 | BRCA2-002 | 13 | 30751977 | 30770409 |
| A4 | OTTHUMT00013000866 | bA37E23.3-001 | 13 | 30756797 | 30758245 |
| A4 | OTTHUMT00013000872 | bA298P3.2-001 | 13 | 30773713 | 30800270 |
| A4 | OTTHUMT00013000873 | bA298P3.2-002 | 13 | 30773810 | 30800270 |
| A4 | OTTHUMT00013000874 | bA298P3.2-003 | 13 | 30773814 | 30788840 |
| A4 | OTTHUMT00013000875 | bA298P3.2-004 | 13 | 30773954 | 30800151 |
| A4 | ENSESTT00000037479 | | 13 | 30774634 | 30800259 |
| A4 | ENSESTT00000037481 | | 13 | 30774983 | 30779922 |
| A4 | ENST00000267044 | NM_052818 | 13 | 30775079 | 30779464 |
| A4 | ENST00000306588 | Q8WTU5 | 13 | 30775281 | 30800219 |
| A4 | OTTHUMT00013000876 | bA298P3.2-005 | 13 | 30775816 | 30800151 |
| A4 | OTTHUMT00013000878 | bA298P3.2-007 | 13 | 30778590 | 30800143 |
| A4 | ENST00000332066 | NM_014081 | 13 | 30779092 | 30779373 |
| A4 | OTTHUMT00013000877 | bA298P3.2-006 | 13 | 30797059 | 30800151 |
| A4 | OTTHUMT00013000879 | bA298P3.2-008 | 13 | 30797495 | 30805091 |
| A4 | ENSESTT00000037480 | | 13 | 30797642 | 30800151 |
| A4 | OTTHUMT00013000888 | bA298P3.3-001 | 13 | 30804554 | 30818619 |
| A4 | ENSESTT00000037478 | | 13 | 30804625 | 30814984 |
| A4 | ENST00000267052 | NM_033111 | 13 | 30804924 | 30818554 |
| A4 | ENSESTT00000037476 | | 13 | 30815994 | 30910936 |
| A4 | OTTHUMT00013000896 | bA11K16.4-001 | 13 | 30816130 | 30816268 |
| A4 | OTTHUMT00013000890 | bA11K16.1-001 | 13 | 30849411 | 30850978 |
| A4 | OTTHUMT00013000892 | bA11K16.2-001 | 13 | 30852732 | 30890057 |
| A4 | OTTHUMT00013000894 | bA11K16.3-001 | 13 | 30864436 | 30870629 |
| A4 | ENST00000267068 | NM_014887 | 13 | 30889031 | 30910919 |
| A4 | OTTHUMT00013000897 | bA11K16.4-002 | 13 | 30889031 | 30910919 |
| A4 | ENSESTT00000037473 | | 13 | 30889508 | 30894409 |
| A4 | ENSESTT00000037471 | | 13 | 30889508 | 30908302 |
| A4 | ENSESTT00000037474 | | 13 | 30889684 | 30893770 |
| A4 | OTTHUMT00013000898 | bA11K16.4-003 | 13 | 30889998 | 30908006 |
| A4 | ENSESTT00000037472 | | 13 | 30899013 | 30908193 |
| A4 | ENSESTT00000037467 | | 13 | 30907172 | 30908872 |
| A4 | ENSESTT00000037477 | | 13 | 30908368 | 30910932 |
| A4 | ENSESTT00000037470 | | 13 | 30908503 | 30910929 |
| A4 | OTTHUMT00013000899 | bA11K16.4-004 | 13 | 30908564 | 30910962 |
| A4 | ENSESTT00000037475 | | 13 | 30908708 | 30910962 |
| A4 | ENSESTT00000037468 | | 13 | 30958592 | 31031343 |
| A4 | OTTHUMT00013000906 | 49J10.1-006 | 13 | 30958624 | 31118238 |
| A4 | ENST00000261578 | APRIN | 13 | 30958642 | 31145465 |
| A4 | OTTHUMT00013000907 | 49J10.1-007 | 13 | 30958685 | 31118238 |
| A4 | OTTHUMT00013000908 | 49J10.1-008 | 13 | 30958688 | 31024465 |
| A4 | ENSESTT00000037469 | | 13 | 30958704 | 31021469 |
| A4 | OTTHUMT00013000904 | 49J10.1-002 | 13 | 30958706 | 31022754 |
| A4 | ENSESTT00000037369 | | 13 | 31071946 | 31113272 |
| A4 | ENSESTT00000037370 | | 13 | 31073121 | 31113272 |
| A4 | OTTHUMT00013000905 | 49J10.1-004 | 13 | 31073219 | 31079179 |
| A4 | ENSESTT00000037371 | | 13 | 31073219 | 31079179 |
| A4 | OTTHUMT00013000915 | bA380B4.1-002 | 13 | 31125470 | 31148043 |
| A4 | OTTHUMT00013000914 | bA380B4.1-001 | 13 | 31125470 | 31150157 |
| A4 | ENSESTT00000037372 | | 13 | 31130670 | 31143217 |
| A4 | OTTHUMT00013000918 | bA380B4.2-001 | 13 | 31249569 | 31283788 |
| A4 | OTTHUMT00013000920 | bA218A18.1-001 | 13 | 31325521 | 31326539 |
| A4 | ENSESTT00000037388 | | 13 | 31385588 | 31388081 |
| A4 | OTTHUMT00013003671 | KL-002 | 13 | 31388571 | 31436440 |
| A4 | ENST00000255481KL | | 13 | 31388571 | 31438279 |
| A4 | OTTHUMT00013003670 | KL-001 | 13 | 31388571 | 31438282 |
| A4 | OTTHUMT00013000926 | bA81F11.1-001 | 13 | 31475278 | 31578143 |
| A4 | ENSESTT00000037373 | | 13 | 31476827 | 31478958 |
| A4 | ENSESTT00000037386 | | 13 | 31477004 | 31484030 |
| A4 | ENST00000255486 | STARD13 | 13 | 31477730 | 31558159 |
| A4 | ENSESTT00000037387 | | 13 | 31482009 | 31484030 |
| A4 | OTTHUMT00013000930 | bA81F11.1-005 | 13 | 31483921 | 31485330 |
| A4 | OTTHUMT00013000924 | bA81F11.4-001 | 13 | 31494713 | 31495797 |
| A4 | ENSESTT00000037374 | | 13 | 31494713 | 31495802 |
| A4 | OTTHUMT00013000928 | bA81F11.1-003 | 13 | 31501869 | 31558216 |
| A4 | ENSESTT00000037379 | | 13 | 31502223 | 31578161 |
| A4 | ENSESTT00000037378 | | 13 | 31502350 | 31722654 |
| A4 | OTTHUMT00013000929 | bA81F11.1-004 | 13 | 31510566 | 31539759 |
| A4 | ENSESTT00000037380 | | 13 | 31510566 | 31539761 |
| A4 | OTTHUMT00013000927 | bA81F11.1-002 | 13 | 31527573 | 31657844 |
| A4 | OTTHUMT00013000922 | bA81F11.2-001 | 13 | 31530724 | 31536546 |
| A4 | ENSESTT00000037385 | | 13 | 31530724 | 31536546 |
| A4 | ENSESTT00000037384 | | 13 | 31547153 | 31657728 |
| A4 | OTTHUMT00013000936 | bA81F11.3-001 | 13 | 31562078 | 31657873 |
| A4 | ENSESTT00000037383 | | 13 | 31647964 | 31657873 |
| A4 | OTTHUMT00013000938 | bA363P13.1-001 | 13 | 31649690 | 31653471 |
| A4 | ENSESTT00000037375 | | 13 | 31649690 | 31653473 |
| A4 | ENSESTT00000037381 | | 13 | 31705816 | 31720135 |
| A4 | OTTHUMT00013000940 | bA141M1.1-001 | 13 | 31705983 | 31707403 |
| A4 | ENSESTT00000037382 | | 13 | 31705983 | 31707414 |
| A4 | OTTHUMT00013000942 | bA141M1.4-001 | 13 | 31720744 | 31722761 |
| A4 | ENSESTT00000037377 | | 13 | 31721064 | 31722742 |
| A4 | OTTHUMT00013000946 | bA141M1.3-001 | 13 | 31727343 | 32048905 |
| A4 | OTTHUMT00013000948 | bA141M1.3-003 | 13 | 31883467 | 31896459 |
| A4 | ENSESTT00000037376 | | 13 | 31883467 | 31896459 |
| A4 | OTTHUMT00013000944 | bA37L2.1-001 | 13 | 31983104 | 31983659 |
| A4 | OTTHUMT00013000947 | bA141M1.3-002 | 13 | 32026796 | 32048861 |
| A4 | OTTHUMT00013000954 | bA179A7.2-001 | 13 | 32029573 | 32032065 |
| A4 | OTTHUMT00013000952 | RFC3-001 | 13 | 32190203 | 32209633 |
| A4 | ENSESTT00000039806 | | 13 | 32190245 | 32209644 |
| A4 | ENSESTT00000039805 | | 13 | 32190245 | 32338382 |
| A4 | ENSESTT00000039804 | | 13 | 32190245 | 32338695 |
| A4 | ENST00000255484 | RFC3 | 13 | 32190316 | 32208432 |
| A4 | OTTHUMT00013000956 | bA218I21.1-001 | 13 | 32454566 | 32455447 |
| A4 | OTTHUMT00013000958 | bA266E6.1-001 | 13 | 32807587 | 33012822 |
| A4 | ENSESTT00000039807 | | 13 | 32807587 | 33012822 |
| A4 | OTTHUMT00013000959 | bA266E6.1-002 | 13 | 32905983 | 32906487 |
| A4 | ENSESTT00000039808 | | 13 | 32905983 | 32906487 |
| A4 | OTTHUMT00013000962 | bA266E6.2-001 | 13 | 32946341 | 32946803 |
| A4 | OTTHUMT00013000972 | NBEA-001 | 13 | 33314456 | 34044873 |
| A4 | ENST00000310336 | NBEA | 13 | 33314958 | 34043128 |
| A5 | ENSESTT00000038767 | | 7 | 101019421 | 101400637 |
| A5 | ENSESTT00000038766 | | 7 | 101019421 | 101485396 |
| A5 | ENSESTT00000038765 | | 7 | 101019421 | 101487431 |
| A5 | OTTHUMT00007006261 | CUTL1 | 7 | 101019473 | 101487362 |
| A5 | ENST00000292538 | CUTL1 | 7 | 101019492 | 101486563 |
| A5 | ENST00000292535 | CUTL1 | 7 | 101021063 | 101453321 |
| A5 | ENSESTT00000038777 | | 7 | 101081303 | 101082106 |
| A5 | ENSESTT00000038768 | | 7 | 101119629 | 101231763 |
| A5 | ENSESTT00000038771 | | 7 | 101273798 | 101400637 |
| A5 | ENSESTT00000038770 | | 7 | 101273798 | 101485396 |
| A5 | ENSESTT00000038769 | | 7 | 101273798 | 101487431 |
| A5 | ENSESTT00000038772 | | 7 | 101362019 | 101397352 |
| A5 | OTTHUMT00007006649 | mbhmh_gw729093.100322718.100376677.3.8e− | 7 | 101400541 | 101405344 |
| A5 | ENSESTT00000038778 | | 7 | 101400655 | 101404987 |
| A5 | OTTHUMT00007007070 | mbhmh_H_RG313A17_F193468.fgenesh2.2 | 7 | 101429721 | 101452503 |
| A5 | OTTHUMT00007007797 | Hs_7_c1557 | 7 | 101468176 | 101468461 |
| A5 | ENSESTT00000038775 | | 7 | 101477627 | 101484340 |
| A5 | ENSESTT00000038774 | | 7 | 101477627 | 101485396 |
| A5 | ENSESTT00000038773 | | 7 | 101477627 | 101487431 |
| A5 | ENSESTT00000038776 | | 7 | 101486181 | 101487431 |
| A5 | ENSESTT00000038779 | | 7 | 101488534 | 101512349 |
| A5 | OTTHUMT00007006892 | mbax_h_100048510_100795952_m_134702119_1 | 7 | 101498891 | 101522278 |
| A5 | ENST00000306803 | NM_020979 | 7 | 101504016 | 101522278 |
| A5 | ENSESTT00000038780 | | 7 | 101517823 | 101522351 |
| A5 | OTTHUMT00007007831 | Hs_7_c1560 | 7 | 101527852 | 101530126 |
| A5 | OTTHUMT00007007835 | Hs_7_c1561 | 7 | 101537904 | 101547742 |
| A5 | OTTHUMT00007006950 | mbhmh_ts.101.008.a | 7 | 101547738 | 101553959 |
| A5 | ENST00000331921 | | 7 | 101548723 | 101555503 |
| A5 | ENST00000332533 | | 7 | 101551346 | 101554016 |
| A5 | ENSESTT00000038782 | | 7 | 101564512 | 101576836 |
| A5 | ENSESTT00000038781 | | 7 | 101564512 | 101581544 |
| A5 | ENSESTT00000038783 | | 7 | 101564790 | 101581544 |
| A5 | OTTHUMT00007007838 | Hs_7_c1564 | 7 | 101564796 | 101586300 |
| A5 | ENSESTT00000038785 | | 7 | 101566265 | 101576836 |
| A5 | ENSESTT00000038784 | | 7 | 101566265 | 101581544 |
| A5 | ENST00000329536 | | 7 | 101576257 | 101576794 |
| A5 | OTTHUMT00007007842 | Hs_7_c1565 | 7 | 101581374 | 101583647 |
| A5 | ENST00000262936 | PRKRIP1 | 7 | 101597276 | 101627593 |
| A5 | ENSESTT00000038786 | | 7 | 101597306 | 101626253 |
| A5 | ENST00000292563 | C7orf19 | 7 | 101634483 | 101649629 |
| A5 | OTTHUMT00007006331 | CBCIP2 | 7 | 101634489 | 101649629 |
| A5 | ENSESTT00000038787 | | 7 | 101634512 | 101647966 |
| A5 | ENSESTT00000038788 | | 7 | 101637128 | 101647966 |
| A5 | OTTHUMT00007006536 | FLJ20013 | 7 | 101657151 | 101665754 |
| A5 | ENST00000292566 | NM_017621 | 7 | 101657178 | 101665774 |
| A5 | ENSESTT00000038837 | | 7 | 101658436 | 101665789 |
| A5 | OTTHUMT00007006660 | mbhmh_nh_h_100048510_100795952_m_1347021 | 7 | 101665582 | 101673962 |
| A5 | ENST00000292616 | NM_152892 | 7 | 101665847 | 101673962 |
| A5 | ENSESTT00000038789 | | 7 | 101665866 | 101669568 |
| A5 | ENSESTT00000038790 | | 7 | 101670502 | 101675765 |
| A5 | ENSESTT00000038791 | | 7 | 101670502 | 101675765 |
| A5 | ENSESTT00000038802 | | 7 | 101674085 | 101770880 |
| A5 | ENSESTT00000038803 | | 7 | 101674197 | 101679846 |
| A5 | OTTHUMT00007006868 | POLR2J | 7 | 101674370 | 101679823 |
| A5 | ENSESTT00000038807 | | 7 | 101674370 | 101869945 |
| A5 | ENST00000292614 | POLR2J | 7 | 101674562 | 101679773 |
| A5 | ENST00000326391 | | 7 | 101675112 | 101679773 |
| A5 | ENSESTT00000038806 | | 7 | 101675384 | 101872600 |
| A5 | OTTHUMT00007006642 | HSPC047.1 | 7 | 101680646 | 101681562 |
| A5 | ENSESTT00000038818 | | 7 | 101684052 | 101884280 |
| A5 | ENSESTT00000038815 | | 7 | 101684387 | 101887718 |
| A5 | ENSESTT00000038813 | | 7 | 101684414 | 101889940 |
| A5 | ENST00000306682 | NM_006989 | 7 | 101684505 | 101718623 |
| A5 | OTTHUMT00007006603 | mbhmh_gw5901958.100606911.100642795.1.6e | 7 | 101684508 | 101718623 |
| A5 | ENSESTT00000038814 | | 7 | 101685947 | 101889940 |
| A5 | ENSESTT00000038819 | | 7 | 101685981 | 101793412 |
| A5 | ENSESTT00000038831 | | 7 | 101691969 | 101794030 |
| A5 | ENSESTT00000038792 | | 7 | 101694343 | 101794786 |
| A5 | ENSESTT00000038793 | | 7 | 101694383 | 101794951 |
| A5 | ENSESTT00000038795 | | 7 | 101694776 | 101695508 |
| A5 | ENSESTT00000038829 | | 7 | 101697050 | 101702049 |
| A5 | ENSESTT00000038828 | | 7 | 101697479 | 101707749 |
| A5 | ENSESTT00000038826 | | 7 | 101697813 | 101801370 |
| A5 | ENSESTT00000038821 | | 7 | 101710309 | 101817727 |
| A5 | ENSESTT00000038822 | | 7 | 101713854 | 101813329 |
| A5 | ENSESTT00000038820 | | 7 | 101724207 | 101823338 |
| A5 | ENSESTT00000038810 | | 7 | 101739241 | 101839744 |
| A5 | ENSESTT00000038808 | | 7 | 101739246 | 101866719 |
| A5 | ENSESTT00000038809 | | 7 | 101739290 | 101866719 |
| A5 | ENST00000297278 | | 7 | 101739330 | 101744501 |
| A5 | ENSESTT00000038804 | | 7 | 101742184 | 101872640 |
| A5 | OTTHUMT00007007119 | POLR2J2 | 7 | 101742452 | 101773584 |
| A5 | ENST00000319405 | | 7 | 101755410 | 101861246 |
| A5 | OTTHUMT00007006426 | mbhmh_H_RG158O17_F218045.fgenesh2.3 | 7 | 101757047 | 101763166 |
| A5 | OTTHUMT00007008004 | Hs_7_c5034 | 7 | 101763162 | 101768147 |
| A5 | ENSESTT00000038796 | | 7 | 101766314 | 101869984 |
| A5 | ENSESTT00000038811 | | 7 | 101768689 | 101773566 |
| A5 | ENST00000323465 | NM_145325 | 7 | 101768862 | 101773534 |
| A5 | OTTHUMT00007006643 | HSPC047.2 | 7 | 101774402 | 101780795 |
| A5 | ENSESTT00000038817 | | 7 | 101783624 | 101884523 |
| A5 | ENSESTT00000038816 | | 7 | 101783749 | 101884558 |
| A5 | ENST00000262940 | RSG5_HUMAN | 7 | 101783754 | 101817670 |
| A5 | OTTHUMT00007006268 | CAPRI | 7 | 101783754 | 101817670 |
| A5 | ENSESTT00000038797 | | 7 | 101793580 | 101890696 |
| A5 | ENSESTT00000038794 | | 7 | 101794005 | 101794745 |
| A5 | ENSESTT00000038827 | | 7 | 101796283 | 101801278 |
| A5 | ENSESTT00000038825 | | 7 | 101796715 | 101806980 |
| A5 | OTTHUMT00007006314 | mbhmh_gw12844788.100760681.100765918.2.7 | 7 | 101838509 | 101841746 |
| A5 | OTTHUMT00007007045 | mbhmh_ts.101.018.a | 7 | 101853330 | 101862231 |
| A5 | ENST00000312297 | | 7 | 101855961 | 101858620 |
| A5 | OTTHUMT00007008008 | Hs_7_c5035 | 7 | 101862227 | 101867194 |
| A5 | ENSESTT00000038805 | | 7 | 101867736 | 101872630 |
| A5 | OTTHUMT00007006195 | mbhmh_h_100048510_100795952_m_134702119_{—} | 7 | 101867909 | 101872582 |
| A5 | OTTHUMT00007007904 | Hs_7_c1585 | 7 | 101879466 | 101879837 |
| A5 | OTTHUMT00007007905 | Hs_7_c1586 | 7 | 101882816 | 101891453 |
| A5 | ENST00000329942 | Q96C79 | 7 | 101889904 | 101890065 |
| A5 | ENSESTT00000038798 | | 7 | 101889915 | 101890655 |
| A5 | OTTHUMT00007007907 | Hs_7_c1587 | 7 | 101904290 | 101904451 |
| A5 | ENSESTT00000038812 | | 7 | 101904439 | 101949674 |
| A5 | ENSESTT00000038799 | | 7 | 101949942 | 101962382 |
| A5 | ENSESTT00000038800 | | 7 | 101949949 | 101988333 |
| A5 | ENST00000314526 | NM_147194 | 7 | 101950186 | 101950617 |
| A5 | ENST00000314157 | | 7 | 101956102 | 101956916 |
| A5 | OTTHUMT00007007908 | Hs_7_c1588 | 7 | 101956129 | 101956907 |
| A5 | OTTHUMT00007006547 | mbhmh_h_100815628_101274767_m_19823891_2 | 7 | 101960818 | 101982855 |
| A5 | ENSESTT00000038801 | | 7 | 101988311 | 102009612 |
| A5 | OTTHUMT00007007909 | Hs_7_c1589 | 7 | 102000041 | 102007712 |
| A5 | OTTHUMT00007006171 | MGC21636 | 7 | 102014212 | 102174163 |
| A5 | ENST00000313221 | NM_145032 | 7 | 102014214 | 102230396 |
| A5 | ENST00000313196 | Q8N1P0 | 7 | 102014320 | 102228534 |
| A5 | ENSESTT00000040069 | | 7 | 102113941 | 102146070 |
| A5 | ENST00000249377 | LRRC17 | 7 | 102113983 | 102145927 |
| A5 | OTTHUMT00007006209 | P37MB | 7 | 102113983 | 102145927 |
| A5 | ENSESTT00000040175 | | 7 | 102127277 | 102174180 |
| A5 | ENSESTT00000040070 | | 7 | 102140423 | 102146070 |
| A5 | ENST00000335370 | Q86UQ8 | 7 | 102174535 | 102177193 |
| A5 | ENSESTT00000040174 | | 7 | 102226175 | 102275821 |
| A5 | OTTHUMT00007007936 | Hs_7_c1593 | 7 | 102270250 | 102270369 |
| A5 | ENSESTT00000040076 | | 7 | 102276116 | 102288294 |
| A5 | ENSESTT00000040075 | | 7 | 102276116 | 102299941 |
| A5 | OTTHUMT00007006404 | MGC3195 | 7 | 102276139 | 102299655 |
| A5 | ENST00000323716 | NM_031905 | 7 | 102276251 | 102299531 |
| A5 | ENST00000306450 | Q8IZC1 | 7 | 102276251 | 102299531 |
| A5 | ENST00000323735 | Q8IZC2 | 7 | 102276251 | 102299531 |
| A5 | ENSESTT00000040171 | | 7 | 102303502 | 102321207 |
| A5 | ENSESTT00000040170 | | 7 | 102303502 | 102329508 |
| A5 | ENSESTT00000040173 | | 7 | 102304193 | 102316319 |
| A5 | ENSESTT00000040172 | | 7 | 102304334 | 102320671 |
| A5 | OTTHUMT00007006927 | mbhmh_h_100815628_101274767_m_19823891_2 | 7 | 102307967 | 102331431 |
| A5 | ENST00000292634 | | 7 | 102316038 | 102321120 |
| A5 | OTTHUMT00007007937 | Hs_7_c1596 | 7 | 102342330 | 102342917 |
| A5 | ENST00000327597 | | 7 | 102342504 | 102342917 |
| A5 | ENST00000333351 | | 7 | 102353043 | 102353323 |
| A5 | OTTHUMT00007007938 | Hs_7_c1597 | 7 | 102353043 | 102353323 |
| A5 | ENSESTT00000040163 | | 7 | 102376120 | 102396451 |
| A5 | ENSESTT00000040164 | | 7 | 102376238 | 102386518 |
| A5 | ENSESTT00000040161 | | 7 | 102376238 | 102411289 |
| A5 | OTTHUMT00007007939 | Hs_7_c1598 | 7 | 102376305 | 102481130 |
| A5 | ENST00000306389 | NM_182634 | 7 | 102398168 | 102444053 |
| A5 | ENSESTT00000040162 | | 7 | 102398194 | 102399966 |
| A5 | ENSESTT00000040160 | | 7 | 102399052 | 102411365 |
| A5 | ENST00000257741 | S100A11P | 7 | 102462978 | 102463286 |
| A5 | OTTHUMT00007006524 | S100A14 | 7 | 102462978 | 102463286 |
| A5 | ENSESTT00000040158 | | 7 | 102478355 | 102481237 |
| A5 | ENST00000320297 | Q8N7T0 | 7 | 102478580 | 102481130 |
| A5 | ENSESTT00000040082 | | 7 | 102498413 | 102511457 |
| A5 | OTTHUMT00007006222 | PMPCB | 7 | 102498424 | 102513390 |
| A5 | ENST00000249269 | PMPCB | 7 | 102498438 | 102513249 |
| A5 | ENSESTT00000040083 | | 7 | 102504818 | 102511457 |
| A5 | ENSESTT00000040095 | | 7 | 102513072 | 102513736 |
| A5 | OTTHUMT00007006445 | mpp11 | 7 | 102513452 | 102545644 |
| A5 | ENSESTT00000040149 | | 7 | 102513455 | 102520658 |
| A5 | ENSESTT00000040147 | | 7 | 102513455 | 102520824 |
| A5 | ENSESTT00000040145 | | 7 | 102513455 | 102528369 |
| A5 | ENSESTT00000040141 | | 7 | 102513455 | 102528679 |
| A5 | ENSESTT00000040142 | | 7 | 102513455 | 102528679 |
| A5 | ENSESTT00000040133 | | 7 | 102513455 | 102545745 |
| A5 | ENSESTT00000040134 | | 7 | 102513455 | 102545745 |
| A5 | ENSESTT00000040135 | | 7 | 102513455 | 102545745 |
| A5 | ENSESTT00000040136 | | 7 | 102513455 | 102545745 |
| A5 | ENSESTT00000040129 | | 7 | 102513455 | 102545791 |
| A5 | ENSESTT00000040130 | | 7 | 102513455 | 102545791 |
| A5 | ENSESTT00000040151 | | 7 | 102513480 | 102514323 |
| A5 | ENSESTT00000040150 | | 7 | 102513495 | 102517068 |
| A5 | ENST00000249270 | ZRF1 | 7 | 102513500 | 102545600 |
| A5 | ENST00000222539 | Q9BVX1 | 7 | 102513547 | 102523062 |
| A5 | ENSESTT00000040148 | | 7 | 102516740 | 102520824 |
| A5 | ENSESTT00000040146 | | 7 | 102517478 | 102528369 |
| A5 | ENSESTT00000040143 | | 7 | 102517478 | 102528679 |
| A5 | ENSESTT00000040144 | | 7 | 102517478 | 102528679 |
| A5 | ENSESTT00000040137 | | 7 | 102517478 | 102545745 |
| A5 | ENSESTT00000040138 | | 7 | 102517478 | 102545745 |
| A5 | ENSESTT00000040139 | | 7 | 102517478 | 102545745 |
| A5 | ENSESTT00000040140 | | 7 | 102517478 | 102545745 |
| A5 | ENSESTT00000040131 | | 7 | 102517478 | 102545791 |
| A5 | ENSESTT00000040132 | | 7 | 102517478 | 102545791 |
| A5 | ENSESTT00000040096 | | 7 | 102548636 | 102563744 |
| A5 | ENSESTT00000040097 | | 7 | 102548652 | 102569791 |
| A5 | ENSESTT00000040098 | | 7 | 102548654 | 102569791 |
| A5 | OTTHUMT00007006130 | PSMC2 | 7 | 102548658 | 102569187 |
| A5 | ENSESTT00000040100 | | 7 | 102548673 | 102565286 |
| A5 | ENSESTT00000040099 | | 7 | 102548673 | 102569791 |
| A5 | ENSESTT00000040101 | | 7 | 102548684 | 102565286 |
| A5 | ENST00000292644 | PSMC2 | 7 | 102548690 | 102569032 |
| A5 | OTTHUMT00007007711 | Hs_7_c1604 | 7 | 102549579 | 102549746 |
| A5 | ENSESTT00000040103 | | 7 | 102556671 | 102565286 |
| A5 | ENSESTT00000040104 | | 7 | 102556671 | 102565286 |
| A5 | ENSESTT00000040102 | | 7 | 102556671 | 102569791 |
| A5 | ENSESTT00000040106 | | 7 | 102562512 | 102565286 |
| A5 | ENSESTT00000040105 | | 7 | 102562512 | 102569791 |
| A5 | OTTHUMT00007007089 | mbhmh_h_101446068_102346067_m_20202649_2 | 7 | 102575377 | 102600632 |
| A5 | ENST00000306312 | PRES_HUMAN | 7 | 102575377 | 102622492 |
| A5 | OTTHUMT00007007256 | RELN | 7 | 102672768 | 103190494 |
| A5 | ENSESTT00000040126 | | 7 | 102673292 | 102684682 |
| A5 | ENSESTT00000040125 | | 7 | 102684757 | 102690743 |
| A5 | ENSESTT00000040124 | | 7 | 102691660 | 102698900 |
| A5 | ENSESTT00000040123 | | 7 | 102699088 | 102716370 |
| A5 | ENSESTT00000040122 | | 7 | 102721238 | 102741291 |
| A5 | ENSESTT00000040121 | | 7 | 102743823 | 102746233 |
| A5 | ENSESTT00000040120 | | 7 | 102795307 | 102812764 |
| A5 | ENSESTT00000040118 | | 7 | 102828853 | 102831012 |
| A5 | ENSESTT00000040117 | | 7 | 102836540 | 102853738 |
| A5 | ENSESTT00000040114 | | 7 | 102898905 | 102924166 |
| A5 | OTTHUMT00007006419 | PRO1598 | 7 | 103134831 | 103136533 |
| A5 | ENST00000314952 | NM_018503 | 7 | 103135443 | 103135619 |
| A5 | ENSESTT00000070876 | | 7 | 103327319 | 103337875 |
| A5 | OTTHUMT00007006177 | ORC5L | 7 | 103327321 | 103408971 |
| A5 | ENST00000297431 | ORC5L | 7 | 103327826 | 103408883 |
| A5 | ENSESTT00000070875 | | 7 | 103368963 | 103389283 |
| A5 | OTTHUMT00007007749 | Hs_7_c1615 | 7 | 103380669 | 103381272 |
| A5 | OTTHUMT00007006936 | mbhmh_h_102246067_103146066_m_21002648_2 | 7 | 103529801 | 103714756 |
| A5 | ENSESTT00000070872 | | 7 | 103529849 | 104107212 |
| A5 | ENST00000330383 | | 7 | 103868727 | 103869859 |
| A5 | OTTHUMT00007007754 | Hs_7_c1617 | 7 | 103868727 | 103870551 |
| A5 | ENSESTT00000070874 | | 7 | 103868774 | 103870161 |
| A5 | OTTHUMT00007007536 | Hs_7_c3016 | 7 | 103871565 | 103871676 |
| A5 | OTTHUMT00007007047 | mbhmh_h_102246067_103146066_m_21002648_2 | 7 | 103887281 | 103938199 |
| A5 | ENSESTT00000070873 | | 7 | 103939577 | 103945127 |
| A5 | OTTHUMT00007006239 | nh_nm_gi16307605 | 7 | 103997484 | 104005062 |
| A5 | OTTHUMT00007007758 | Hs_7_c1619 | 7 | 104027314 | 104027771 |
| A5 | ENSESTT00000041852 | | 7 | 104142399 | 104163317 |
| A5 | OTTHUMT00007007780 | Hs_7_c1620 | 7 | 104191028 | 104191180 |
| A5 | ENSESTT00000041867 | | 7 | 104215182 | 104242044 |
| A5 | ENSESTT00000041866 | | 7 | 104215182 | 104263257 |
| A5 | ENSESTT00000041864 | | 7 | 104215182 | 104265008 |
| A5 | ENSESTT00000041865 | | 7 | 104215182 | 104265008 |
| A5 | ENSESTT00000041862 | | 7 | 104215182 | 104275754 |
| A5 | ENSESTT00000041863 | | 7 | 104215182 | 104275754 |
| A5 | ENST00000311117 | MLL5 | 7 | 104215186 | 104314311 |
| A5 | ENSESTT00000041854 | | 7 | 104215192 | 104242044 |
| A5 | ENSESTT00000041853 | | 7 | 104215192 | 104263257 |
| A5 | ENSESTT00000041868 | | 7 | 104215192 | 104275754 |
| A5 | ENSESTT00000041855 | | 7 | 104215403 | 104275754 |
| A5 | OTTHUMT00007006515 | MLL5 | 7 | 104241847 | 104302458 |
| A5 | ENST00000257745 | O95038 | 7 | 104241931 | 104301566 |
| A5 | ENST00000333597 | Q8IWR5 | 7 | 104241931 | 104314311 |
| A5 | ENSESTT00000041857 | | 7 | 104264328 | 104303627 |
| A5 | ENSESTT00000041856 | | 7 | 104264328 | 104307572 |
| A5 | ENST00000222422 | Q9NS29 | 7 | 104264368 | 104291189 |
| A5 | ENSESTT00000041858 | | 7 | 104267501 | 104275739 |
| A5 | ENSESTT00000041859 | | 7 | 104278279 | 104291101 |
| A5 | ENST00000334914 | Q86W16 | 7 | 104278380 | 104290339 |
| A5 | ENSESTT00000041860 | | 7 | 104279822 | 104291098 |
| A5 | ENSESTT00000041861 | | 7 | 104302560 | 104307572 |
| A5 | ENST00000249297 | Q86W12 | 7 | 104302864 | 104308193 |
| A5 | ENSESTT00000041870 | | 7 | 104307682 | 104310494 |
| A5 | ENSESTT00000041869 | | 7 | 104307682 | 104313681 |
| A5 | OTTHUMT00007007782 | Hs_7_c1623 | 7 | 104308132 | 104313327 |
| A5 | ENSESTT00000041872 | | 7 | 104308136 | 104310494 |
| A5 | ENSESTT00000041871 | | 7 | 104308136 | 104313681 |
| A5 | ENSESTT00000041873 | | 7 | 104308939 | 104313681 |
| A5 | ENST00000334884 | Q86TI3 | 7 | 104310018 | 104311845 |
| A5 | ENSESTT00000041909 | | 7 | 104311672 | 104318457 |
| A5 | ENST00000334877 | Q86WG0 | 7 | 104312977 | 104314348 |
| A5 | OTTHUMT00007006319 | SRPK2 | 7 | 104317346 | 104469993 |
| A5 | ENST00000257701 | SRPK2 | 7 | 104317350 | 104470008 |
| A5 | ENSESTT00000041904 | | 7 | 104318221 | 104470012 |
| A5 | ENSESTT00000041900 | | 7 | 104318221 | 104589682 |
| A5 | ENSESTT00000041896 | | 7 | 104318221 | 104589703 |
| A5 | ENSESTT00000041910 | | 7 | 104318221 | 104589839 |
| A5 | ENSESTT00000041908 | | 7 | 104318327 | 104404634 |
| A5 | ENSESTT00000041905 | | 7 | 104318558 | 104470012 |
| A5 | ENSESTT00000041901 | | 7 | 104318558 | 104589682 |
| A5 | ENSESTT00000041897 | | 7 | 104318558 | 104589703 |
| A5 | ENSESTT00000041911 | | 7 | 104318558 | 104589839 |
| A5 | ENSESTT00000041906 | | 7 | 104346254 | 104470012 |
| A5 | ENSESTT00000041902 | | 7 | 104346254 | 104589682 |
| A5 | ENSESTT00000041898 | | 7 | 104346254 | 104589703 |
| A5 | ENSESTT00000041894 | | 7 | 104346254 | 104589839 |
| A5 | ENSESTT00000041907 | | 7 | 104361279 | 104470012 |
| A5 | ENSESTT00000041903 | | 7 | 104361279 | 104589682 |
| A5 | ENSESTT00000041899 | | 7 | 104361279 | 104589703 |
| A5 | ENSESTT00000041895 | | 7 | 104361279 | 104589839 |
| A5 | OTTHUMT00007007787 | Hs_7_c1625 | 7 | 104390180 | 104390804 |
| A5 | OTTHUMT00007007788 | Hs_7_c1626 | 7 | 104405611 | 104405835 |
| A5 | OTTHUMT00007007791 | Hs_7_c1627 | 7 | 104445633 | 104445945 |
| A5 | OTTHUMT00007007792 | Hs_7_c1628 | 7 | 104502500 | 104503061 |
| A5 | OTTHUMT00007007794 | Hs_7_c1629 | 7 | 104555221 | 104555496 |
| A5 | ENST00000257687 | NM_019042 | 7 | 104657493 | 104709424 |
| A5 | ENSESTT00000041893 | | 7 | 104658206 | 104660239 |
| A5 | ENSESTT00000041892 | | 7 | 104658241 | 104707036 |
| A5 | OTTHUMT00007006894 | FLJ20485 | 7 | 104658637 | 104723192 |
| A5 | ENSESTT00000041890 | | 7 | 104683290 | 104723218 |
| A5 | ENSESTT00000041891 | | 7 | 104706926 | 104723218 |
| A5 | ENST00000320648 | | 7 | 104731187 | 104731649 |
| A5 | ENSESTT00000041876 | | 7 | 104733157 | 104743633 |
| A5 | ENSESTT00000041875 | | 7 | 104733157 | 104764927 |
| A5 | ENSESTT00000041874 | | 7 | 104733157 | 104766577 |
| A5 | ENST00000257700 | NM_021930 | 7 | 104733207 | 104768660 |
| A5 | ENSESTT00000041877 | | 7 | 104743218 | 104747974 |
| A5 | OTTHUMT00007006406 | FLJ11785 | 7 | 104751134 | 104768660 |
| A5 | ENSESTT00000041887 | | 7 | 104766086 | 104770452 |
| A5 | ENSESTT00000041888 | | 7 | 104766102 | 104770414 |
| A5 | ENSESTT00000041886 | | 7 | 104766102 | 104770466 |
| A5 | ENSESTT00000041889 | | 7 | 104766110 | 104770281 |
| A5 | ENSESTT00000041885 | | 7 | 104768394 | 104782425 |
| A5 | ENSESTT00000041884 | | 7 | 104768394 | 104782668 |
| A5 | ENST00000327788 | | 7 | 104768432 | 104782432 |
| A5 | ENST00000310149 | | 7 | 104783163 | 104784060 |
| A5 | ENST00000329090 | | 7 | 104783236 | 104784089 |
| A5 | ENST00000332220 | | 7 | 104783236 | 104784134 |
| A5 | OTTHUMT00007007834 | Hs_7_c1634 | 7 | 104783239 | 104784179 |
| A5 | OTTHUMT00007006590 | mbhmh_h_103713457_104613456_m_26903715_2 | 7 | 104792229 | 104844029 |
| A5 | ENSESTT00000041883 | | 7 | 104808623 | 104811530 |
| A5 | ENST00000297416 | YC18_HUMAN | 7 | 104808830 | 105077559 |
| A5 | ENST00000275664 | Q9BTQ8 | 7 | 104814454 | 104825244 |
| A5 | ENSESTT00000041880 | | 7 | 104815496 | 104893101 |
| A5 | ENSESTT00000041878 | | 7 | 104816938 | 104818834 |
| A5 | ENSESTT00000041881 | | 7 | 104818847 | 104893101 |
| A5 | ENSESTT00000041882 | | 7 | 104321070 | 104893101 |
| A5 | ENSESTT00000041879 | | 7 | 104961923 | 105077563 |
| A5 | ENST00000318724 | NM_152749 | 7 | 104962355 | 105077535 |
| A5 | OTTHUMT00007007007 | mbhmh_nh_h_103713457_104613456_m_2690371 | 7 | 104989335 | 104993681 |
| A5 | OTTHUMT00007007837 | Hs_7_c1637 | 7 | 105020472 | 105021075 |
| A5 | ENST00000329846 | | 7 | 105020547 | 105021078 |
| A5 | ENSESTT00000035755 | | 7 | 105076067 | 105077551 |
| A5 | OTTHUMT00007007841 | Hs_7_c1638 | 7 | 105076766 | 105083558 |
| A5 | OTTHUMT00007006283 | mbhmh_h_103713457_104613456_m_26103716_2 | 7 | 105095260 | 105151278 |
| A5 | OTTHUMT00007006180 | mbhmh_H_DJ0568B10_F020305.fgenesh2.1 | 7 | 105175823 | 105223819 |
| A5 | ENSESTT00000035734 | | 7 | 105181971 | 105197312 |
| A5 | ENSESTT00000035735 | | 7 | 105202525 | 105205801 |
| A5 | ENST00000317716 | NM_152750 | 7 | 105205474 | 105231797 |
| A5 | ENSESTT00000035736 | | 7 | 105205482 | 105221463 |
| A5 | OTTHUMT00007006497 | mbhmh_nh_h_103713457_104613456_m_2690371 | 7 | 105224488 | 105232430 |
| A5 | ENSESTT00000035737 | | 7 | 105229534 | 105234760 |
| A5 | OTTHUMT00007006260 | SYPL | 7 | 105291482 | 105313528 |
| A5 | ENST00000011473 | SYPL | 7 | 105291489 | 105313588 |
| A5 | ENSESTT00000035754 | | 7 | 105292251 | 105294130 |
| A5 | ENSESTT00000035753 | | 7 | 105292251 | 105298877 |
| A5 | ENSESTT00000035750 | | 7 | 105292251 | 105313289 |
| A5 | ENSESTT00000035752 | | 7 | 105292673 | 105300262 |
| A5 | ENSESTT00000035751 | | 7 | 105300133 | 105313289 |
| A5 | OTTHUMT00007007872 | Hs_7_c1643 | 7 | 105326309 | 105327674 |
| A5 | OTTHUMT00007006155 | PBEF | 7 | 105451187 | 105485888 |
| A5 | ENSESTT00000035747 | | 7 | 105451529 | 105470301 |
| A5 | ENSESTT00000035746 | | 7 | 105451529 | 105471257 |
| A5 | ENSESTT00000035745 | | 7 | 105451529 | 105476028 |
| A5 | ENSESTT00000035740 | | 7 | 105451529 | 105486169 |
| A5 | ENSESTT00000035741 | | 7 | 105451529 | 105486169 |
| A5 | ENSESTT00000035738 | | 7 | 105451529 | 105486589 |
| A5 | ENSESTT00000035749 | | 7 | 105452034 | 105454595 |
| A5 | ENST00000222553 | PBEF_HUMAN | 7 | 105452060 | 105485898 |
| A5 | ENSESTT00000035748 | | 7 | 105462192 | 105464474 |
| A5 | ENSESTT00000035744 | | 7 | 105469472 | 105485522 |
| A5 | ENSESTT00000035742 | | 7 | 105473329 | 105486169 |
| A5 | ENSESTT00000035743 | | 7 | 105473329 | 105486169 |
| A5 | ENSESTT00000035739 | | 7 | 105473329 | 105486589 |
| A5 | OTTHUMT00007007874 | Hs_7_c1646 | 7 | 105516202 | 105516577 |
| A5 | OTTHUMT00007007875 | Hs_7_c1647 | 7 | 105831822 | 105832343 |
| A5 | OTTHUMT00007007586 | Hs_7_c3104 | 7 | 105832534 | 105833055 |
| A5 | OTTHUMT00007006262 | mbxx_nh_chr7.105.006.a | 7 | 105833369 | 105833704 |
| A5 | ENST00000315965 | NM_175884 | 7 | 105861166 | 105861861 |
| A5 | OTTHUMT00007007876 | Hs_7_c1648 | 7 | 105861484 | 105861765 |
| A6 | ENSESTT00000021279 | | 10 | 73471701 | 73512400 |
| A6 | ENST00000311182 | CBARA1 | 10 | 73471701 | 73730468 |
| A6 | ENSESTT00000021278 | | 10 | 73472141 | 73579660 |
| A6 | ENSESTT00000021277 | | 10 | 73581559 | 73730469 |
| A6 | ENSESTT00000021276 | | 10 | 73637423 | 73730476 |
| A6 | ENSESTT00000021271 | | 10 | 73668375 | 73670920 |
| A6 | ENST00000313314 | NM_138357 | 10 | 73796492 | 73992053 |
| A6 | ENST00000286508 | Q96FL3 | 10 | 73796537 | 73990183 |
| A6 | ENSESTT00000021273 | | 10 | 73797084 | 73988744 |
| A6 | ENSESTT00000021272 | | 10 | 73797084 | 73992055 |
| A6 | ENSESTT00000021275 | | 10 | 73964348 | 73988744 |
| A6 | ENSESTT00000021274 | | 10 | 73964348 | 73992055 |
| A6 | ENST00000260885 | NM_152635 | 10 | 73997942 | 74037390 |
| A6 | ENST00000334011 | Q8WWZ8 | 10 | 73998160 | 74036885 |
| A6 | ENSESTT00000021456 | | 10 | 74016118 | 74028872 |
| A6 | ENSESTT00000021457 | | 10 | 74034940 | 74046965 |
| A6 | ENSESTT00000021528 | | 10 | 74039868 | 74058975 |
| A6 | ENST00000260878 | PLA2G13 | 10 | 74039978 | 74059139 |
| A6 | ENST00000332968 | | 10 | 74110192 | 74110956 |
| A6 | ENST00000263556 | P4HA1 | 10 | 74112583 | 74179244 |
| A6 | ENST00000307116 | P4HA1 | 10 | 74112583 | 74179244 |
| A6 | ENSESTT00000021526 | | 10 | 74118611 | 74155608 |
| A6 | ENSESTT00000021527 | | 10 | 74121207 | 74155454 |
| A6 | ENSESTT00000021525 | | 10 | 74157783 | 74201227 |
| A6 | ENST00000299408 | NUDT13 | 10 | 74214816 | 74236183 |
| A6 | ENSESTT00000021458 | | 10 | 74214883 | 74236720 |
| A6 | ENST00000335635 | Q9Y3X2 | 10 | 74226553 | 74235264 |
| A6 | ENST00000325946 | NUDT13 | 10 | 74229464 | 74229628 |
| A6 | ENST00000263565 | SGT1_HUMAN | 10 | 74238886 | 74272420 |
| A6 | ENSESTT00000021521 | | 10 | 74243730 | 74272456 |
| A6 | ENSESTT00000021522 | | 10 | 74258724 | 74272456 |
| A6 | ENSESTT00000021524 | | 10 | 74260633 | 74272402 |
| A6 | ENSESTT00000021523 | | 10 | 74260633 | 74272456 |
| A6 | ENST00000242505 | Q9Y2I0 | 10 | 74279052 | 74346541 |
| A6 | ENSESTT00000021459 | | 10 | 74332501 | 74340393 |
| A6 | ENSESTT00000021460 | | 10 | 74332502 | 74345507 |
| A6 | ENSESTT00000021461 | | 10 | 74334657 | 74340393 |
| A6 | ENSESTT00000021463 | | 10 | 74338355 | 74338793 |
| A6 | ENSESTT00000021462 | | 10 | 74339356 | 74345515 |
| A6 | ENST00000299416 | DNAJC9 | 10 | 74347761 | 74351550 |
| A6 | ENST00000299418 | MRPS16 | 10 | 74353204 | 74357012 |
| A6 | ENSESTT00000021464 | | 10 | 74353210 | 74355109 |
| A6 | ENSESTT00000021465 | | 10 | 74357530 | 74358710 |
| A6 | ENSESTT00000021466 | | 10 | 74357561 | 74358710 |
| A6 | ENSESTT00000021520 | | 10 | 74358121 | 74379898 |
| A6 | ENST00000310715 | NM_145170 | 10 | 74358127 | 74463115 |
| A6 | ENST00000286530 | Q8N7D5 | 10 | 74358336 | 74397767 |
| A6 | ENST00000277916 | ANXA7 | 10 | 74479811 | 74518414 |
| A6 | ENST00000260852 | ANXA7 | 10 | 74480456 | 74505217 |
| A6 | ENSESTT00000021505 | | 10 | 74484518 | 74518436 |
| A6 | ENSESTT00000021506 | | 10 | 74484518 | 74518436 |
| A6 | ENSESTT00000021507 | | 10 | 74484518 | 74518436 |
| A6 | ENSESTT00000021467 | | 10 | 74485833 | 74487824 |
| A6 | ENSESTT00000021517 | | 10 | 74487551 | 74518431 |
| A6 | ENSESTT00000021508 | | 10 | 74487551 | 74518436 |
| A6 | ENSESTT00000021509 | | 10 | 74487551 | 74518436 |
| A6 | ENSESTT00000021518 | | 10 | 74487935 | 74518431 |
| A6 | ENSESTT00000021510 | | 10 | 74487935 | 74518436 |
| A6 | ENSESTT00000021511 | | 10 | 74487935 | 74518436 |
| A6 | ENSESTT00000021512 | | 10 | 74487935 | 74518436 |
| A6 | ENSESTT00000021513 | | 10 | 74487935 | 74518436 |
| A6 | ENSESTT00000021514 | | 10 | 74487935 | 74518436 |
| A6 | ENSESTT00000021519 | | 10 | 74487963 | 74518419 |
| A6 | ENSESTT00000021515 | | 10 | 74487976 | 74518436 |
| A6 | ENSESTT00000021516 | | 10 | 74492701 | 74518436 |
| A6 | ENST00000299432 | ZMYND17 | 10 | 74528914 | 74537973 |
| A6 | ENST00000265920 | PPP3CB | 10 | 74541167 | 74600362 |
| A6 | ENSESTT00000021500 | | 10 | 74542284 | 74575867 |
| A6 | ENSESTT00000021501 | | 10 | 74542284 | 74575867 |
| A6 | ENSESTT00000021502 | | 10 | 74542546 | 74575867 |
| A6 | ENSESTT00000021504 | | 10 | 74542559 | 74571979 |
| A6 | ENST00000320361 | Q8N3W4 | 10 | 74542603 | 74600250 |
| A6 | ENSESTT00000021503 | | 10 | 74548717 | 74575867 |
| A6 | ENSESTT00000021499 | | 10 | 74579279 | 74600362 |
| A6 | ENSESTT00000021468 | | 10 | 74600104 | 74601061 |
| A6 | ENST00000319786 | NM_152586 | 10 | 74601901 | 74621672 |
| A6 | ENSESTT00000021469 | | 10 | 74634518 | 74635149 |
| A6 | ENST00000318330 | MYOZ1 | 10 | 74736016 | 74746118 |
| A6 | ENST00000332382 | | 10 | 74736345 | 74742281 |
| A6 | ENST00000299404 | NM_024875 | 10 | 74749893 | 74755390 |
| A6 | ENST00000310381 | | 10 | 74778960 | 74802116 |
| A6 | ENSESTT00000021498 | | 10 | 74787103 | 74799146 |
| A6 | ENSESTT00000021470 | | 10 | 74834923 | 74835765 |
| A6 | ENST00000333366 | | 10 | 74836264 | 74838018 |
| A6 | ENST00000332341 | | 10 | 74836264 | 74838018 |
| A6 | ENSESTT00000021472 | | 10 | 74848734 | 74869952 |
| A6 | ENSESTT00000021471 | | 10 | 74848734 | 74875148 |
| A6 | ENSESTT00000021473 | | 10 | 74848778 | 74856191 |
| A6 | ENST00000313749 | SEC24C | 10 | 74851194 | 74875548 |
| A6 | ENSESTT00000021474 | | 10 | 74870160 | 74875148 |
| A6 | ENSESTT00000021475 | | 10 | 74872779 | 74875148 |
| A6 | ENSESTT00000021476 | | 10 | 74873363 | 74874530 |
| A6 | ENST00000326248 | NM_173540 | 10 | 74876477 | 74880577 |
| A6 | ENST00000299593 | Q96CJ6 | 10 | 74876947 | 74877339 |
| A6 | ENSESTT00000021478 | | 10 | 74877622 | 74880515 |
| A6 | ENSESTT00000021477 | | 10 | 74877622 | 74883436 |
| A6 | ENSESTT00000021479 | | 10 | 74886411 | 74888015 |
| A6 | ENST00000310182 | Q96BP2 | 10 | 74886424 | 74887727 |
| A6 | ENSESTT00000021480 | | 10 | 74886683 | 74888015 |
| A6 | ENST00000326185 | Q8NB34 | 10 | 74890240 | 74890617 |
| A6 | ENSESTT00000021481 | | 10 | 74893032 | 74894348 |
| A6 | ENSESTT00000021482 | | 10 | 74893456 | 74894353 |
| A6 | ENSESTT00000021483 | | 10 | 74893863 | 74894617 |
| A6 | ENST00000242558 | O94987 | 10 | 74894547 | 74903962 |
| A6 | ENSESTT00000021484 | | 10 | 74895484 | 74896595 |
| A6 | ENSESTT00000021485 | | 10 | 74897161 | 74898597 |
| A6 | ENST00000310153 | Q9H8F3 | 10 | 74897187 | 74906153 |
| A6 | ENSESTT00000021486 | | 10 | 74900732 | 74903751 |
| A6 | ENSESTT00000021487 | | 10 | 74900875 | 74903751 |
| A6 | ENSESTT00000021488 | | 10 | 74904375 | 74906153 |
| A6 | ENST00000325890 | Q8N420 | 10 | 74904393 | 74905880 |
| A6 | ENSESTT00000021489 | | 10 | 74905268 | 74906153 |
| A6 | ENST00000299641 | NDST2 | 10 | 74906812 | 74913092 |
| A6 | ENST00000309979 | Q8WV68 | 10 | 74915239 | 74916144 |
| A6 | ENST00000309967 | | 10 | 74916017 | 74916304 |
| A6 | ENST00000322680 | CAMK2G | 10 | 74916862 | 74978941 |
| A6 | ENST00000305762 | Q8NIA4 | 10 | 74919376 | 74977443 |
| A6 | ENST00000322635 | NM_172169 | 10 | 74919376 | 74978822 |
| A6 | ENST00000277853 | NM_001222 | 10 | 74919376 | 74978822 |
| A6 | ENSESTT00000021493 | | 10 | 74919386 | 74978883 |
| A6 | ENSESTT00000021490 | | 10 | 74919386 | 74978935 |
| A6 | ENSESTT00000021497 | | 10 | 74919578 | 74951712 |
| A6 | ENSESTT00000021495 | | 10 | 74921388 | 74978836 |
| A6 | ENSESTT00000021494 | | 10 | 74921388 | 74978883 |
| A6 | ENSESTT00000021491 | | 10 | 74921388 | 74978935 |
| A6 | ENSESTT00000021496 | | 10 | 74921432 | 74952651 |
| A6 | ENSESTT00000021492 | | 10 | 74953412 | 74978891 |
| A6 | ENSESTT00000021181 | | 10 | 75013538 | 75019592 |
| A6 | ENSESTT00000021180 | | 10 | 75013538 | 75019765 |
| A6 | ENSESTT00000021179 | | 10 | 75013538 | 75021858 |
| A6 | ENST00000317358 | Q8NAK4 | 10 | 75014337 | 75016591 |
| A6 | ENSESTT00000021174 | | 10 | 75015493 | 75019592 |
| A6 | ENSESTT00000021183 | | 10 | 75015493 | 75019765 |
| A6 | ENSESTT00000021182 | | 10 | 75015493 | 75021858 |
| A6 | ENST00000242464 | PLAU | 10 | 75015909 | 75020926 |
| A6 | ENSESTT00000021176 | | 10 | 75016395 | 75019765 |
| A6 | ENSESTT00000021175 | | 10 | 75016395 | 75021858 |
| A6 | ENSESTT00000021178 | | 10 | 75017648 | 75019765 |
| A6 | ENSESTT00000021177 | | 10 | 75017648 | 75021858 |
| A6 | ENSESTT00000021185 | | 10 | 75102475 | 75175189 |
| A6 | ENSESTT00000021184 | | 10 | 75102475 | 75177160 |
| A6 | ENSESTT00000021186 | | 10 | 75102477 | 75168197 |
| A6 | ENST00000277829 | NM_003373 | 10 | 75102569 | 75222530 |
| A6 | ENST00000211998 | VCL | 10 | 75102569 | 75224513 |
| A6 | ENSESTT00000021187 | | 10 | 75147443 | 75177116 |
| A6 | ENSESTT00000021189 | | 10 | 75179224 | 75219023 |
| A6 | ENSESTT00000021188 | | 10 | 75179224 | 75223339 |
| A6 | ENSESTT00000021209 | | 10 | 75200527 | 75206788 |
| A6 | ENSESTT00000021191 | | 10 | 75201559 | 75219023 |
| A6 | ENSESTT00000021190 | | 10 | 75201559 | 75223339 |
| A6 | ENSESTT00000021192 | | 10 | 75218050 | 75223339 |
| A6 | ENSESTT00000021193 | | 10 | 75219116 | 75222883 |
| A6 | ENSESTT00000021208 | | 10 | 75222927 | 75224394 |
| A6 | ENST00000330581 | AP3M1 | 10 | 75226134 | 75255181 |
| A6 | ENSESTT00000021206 | | 10 | 75227787 | 75255426 |
| A6 | ENST00000323546 | NM_030970 | 10 | 75229527 | 75229598 |
| A6 | ENSESTT00000021207 | | 10 | 75233235 | 75234365 |
| A6 | ENSESTT00000021195 | | 10 | 75255568 | 75632868 |
| A6 | ENSESTT00000021194 | | 10 | 75255568 | 75813656 |
| A6 | ENSESTT00000021196 | | 10 | 75255596 | 75328956 |
| A6 | ENST00000286621 | ADK | 10 | 75255640 | 75812806 |
| A6 | ENSESTT00000021199 | | 10 | 75280904 | 75632868 |
| A6 | ENSESTT00000021198 | | 10 | 75280904 | 75704867 |
| A6 | ENSESTT00000021197 | | 10 | 75280904 | 75813656 |
| A6 | ENST00000330453 | | 10 | 75527848 | 75528380 |
| A6 | ENST00000326278 | | 10 | 75631945 | 75632449 |
| A6 | ENSESTT00000021205 | | 10 | 75903848 | 75958752 |
| A6 | ENSESTT00000021201 | | 10 | 75929925 | 75947950 |
| A6 | ENSESTT00000021200 | | 10 | 75929925 | 76076904 |
| A6 | ENST00000287239 | MYST4 | 10 | 75930980 | 76135407 |
| A6 | ENSESTT00000021203 | | 10 | 75930982 | 75947950 |
| A6 | ENSESTT00000021202 | | 10 | 75930982 | 76076904 |
| A6 | ENSESTT00000021204 | | 10 | 75931001 | 75947270 |
| A6 | ENSESTT00000021123 | | 10 | 76083627 | 76086278 |
| A6 | ENSESTT00000021143 | | 10 | 76124157 | 76126508 |
| A6 | ENSESTT00000021142 | | 10 | 76198793 | 76199990 |
| A6 | ENST00000308475 | | 10 | 76198802 | 76213537 |
| A6 | ENSESTT00000021141 | | 10 | 76198943 | 76200127 |
| A6 | ENSESTT00000021139 | | 10 | 76202071 | 76213531 |
| A6 | ENSESTT00000021140 | | 10 | 76208362 | 76213559 |
| A6 | ENST00000330673 | Q96J67 | 10 | 76210030 | 76213518 |
| A6 | ENSESTT00000021124 | | 10 | 76215992 | 76255437 |
| A6 | ENSESTT00000021138 | | 10 | 76228974 | 76230298 |
| A6 | ENST00000287258 | NM_144660 | 10 | 76254890 | 76280615 |
| A6 | ENSESTT00000021125 | | 10 | 76280444 | 76281496 |
| A6 | ENSESTT00000021127 | | 10 | 76314523 | 76322599 |
| A6 | ENSESTT00000021126 | | 10 | 76314523 | 76335808 |
| A6 | ENST00000298468 | VDAC2 | 10 | 76315209 | 76335638 |
| A6 | ENSESTT00000021128 | | 10 | 76315395 | 76335808 |
| A6 | ENST00000304595 | VDAC2 | 10 | 76315945 | 76335465 |
| A6 | ENSESTT00000021129 | | 10 | 76333671 | 76335638 |
| A6 | ENSESTT00000021136 | | 10 | 76338330 | 76339540 |
| A6 | ENSESTT00000021137 | | 10 | 76338332 | 76339540 |
| A6 | ENSESTT00000021135 | | 10 | 76338332 | 76339908 |
| A6 | ENST00000298482 | NM_144589 | 10 | 76338434 | 76340289 |
| A6 | ENSESTT00000021134 | | 10 | 76340085 | 76340318 |
| A6 | ENST00000308111 | NM_032772 | 10 | 76502522 | 76506032 |
| A6 | ENSESTT00000021133 | | 10 | 76504121 | 76505779 |
| A6 | ENSESTT00000021130 | | 10 | 76505464 | 76511780 |
| A6 | ENST00000321905 | | 10 | 76507413 | 76512179 |
| A6 | ENSESTT00000021131 | | 10 | 76508372 | 76511814 |
| A6 | ENST00000260908 | Q9P1K6 | 10 | 76683657 | 76683884 |
| A6 | ENSESTT00000021132 | | 10 | 76875127 | 77140490 |
| A6 | ENST00000277847 | NM_032024 | 10 | 77140367 | 77163144 |
| A6 | ENSESTT00000021106 | | 10 | 77387031 | 77389445 |
| A6 | ENSESTT00000021100 | | 10 | 77428760 | 77661738 |
| A6 | ENSESTT00000021101 | | 10 | 77463793 | 77661593 |
| A6 | ENSESTT00000021098 | | 10 | 77507582 | 77661714 |
| A6 | ENSESTT00000021099 | | 10 | 77569175 | 77661701 |
| A6 | ENSESTT00000021105 | | 10 | 77981962 | 77992271 |
| A6 | ENST00000331566 | | 10 | 77982100 | 77982255 |
| A6 | ENST00000286628 | KCNMA1 | 10 | 77991627 | 78214698 |
| A6 | ENSESTT00000021104 | | 10 | 77991650 | 78019414 |
| A6 | ENSESTT00000021102 | | 10 | 77992403 | 78006633 |
| A6 | ENSESTT00000021103 | | 10 | 77992437 | 78082885 |
| A6 | ENSESTT00000021145 | | 10 | 78009186 | 78053824 |
| A6 | ENSESTT00000021172 | | 10 | 78014424 | 78053629 |
| A6 | ENSESTT00000021173 | | 10 | 78018433 | 78049275 |
| A6 | ENSESTT00000021171 | | 10 | 78053673 | 78116388 |
| A6 | ENSESTT00000021170 | | 10 | 78078354 | 78123424 |
| A6 | ENSESTT00000021168 | | 10 | 78099639 | 78143996 |
| A6 | ENSESTT00000021169 | | 10 | 78116559 | 78143977 |
| A6 | ENSESTT00000021167 | | 10 | 78177554 | 78212924 |
| A6 | ENSESTT00000021166 | | 10 | 78189055 | 78287832 |
| A6 | ENSESTT00000021146 | | 10 | 78204890 | 78205770 |
| A6 | ENSESTT00000021147 | | 10 | 78252653 | 78254461 |
| A6 | ENSESTT00000021162 | | 10 | 78287850 | 78742046 |
| A6 | ENSESTT00000021165 | | 10 | 78686459 | 78723017 |
| A6 | ENSESTT00000021163 | | 10 | 78740556 | 78741848 |
| A6 | ENSESTT00000021164 | | 10 | 78740566 | 78741829 |
| A6 | ENST00000334073 | | 10 | 78836952 | 78838490 |
| A6 | ENST00000320599 | | 10 | 78884695 | 78886237 |
| A6 | ENST00000320511 | DLG5 | 10 | 78895154 | 78961260 |
| A6 | ENSESTT00000021157 | | 10 | 78896519 | 78910150 |
| A6 | ENSESTT00000021161 | | 10 | 78896672 | 78898481 |
| A6 | ENSESTT00000021158 | | 10 | 78896672 | 78910150 |
| A6 | ENSESTT00000021159 | | 10 | 78898010 | 78910150 |
| A6 | ENSESTT00000021160 | | 10 | 78899194 | 78910150 |
| A6 | ENSESTT00000021155 | | 10 | 78911119 | 78913967 |
| A6 | ENSESTT00000021156 | | 10 | 78911160 | 78912313 |
| A6 | ENSESTT00000021154 | | 10 | 78915532 | 78916696 |
| A6 | ENSESTT00000021153 | | 10 | 78920387 | 78923893 |
| A6 | ENSESTT00000021151 | | 10 | 78925896 | 78935239 |
| A6 | ENSESTT00000021152 | | 10 | 78925896 | 78935239 |
| A6 | ENSESTT00000021149 | | 10 | 78925896 | 78946262 |
| A6 | ENSESTT00000021150 | | 10 | 78925896 | 78946262 |
| A6 | ENSESTT00000021148 | | 10 | 78948038 | 78958638 |
| A6 | ENST00000318641 | | 10 | 79032042 | 79032499 |
| A6 | ENST00000277783 | RPC1_HUMAN | 10 | 79080509 | 79133862 |
| A6 | ENSESTT00000021121 | | 10 | 79081352 | 79090348 |
| A6 | ENSESTT00000021122 | | 10 | 79086568 | 79089703 |
| A6 | ENSESTT00000021120 | | 10 | 79104456 | 79128979 |
| A6 | ENSESTT00000021112 | | 10 | 79138153 | 79141824 |
| A6 | ENSESTT00000021111 | | 10 | 79138153 | 79141833 |
| A6 | ENSESTT00000021110 | | 10 | 79138153 | 79144586 |
| A6 | ENSESTT00000021109 | | 10 | 79138153 | 79145062 |
| A6 | ENSESTT00000021108 | | 10 | 79138153 | 79153449 |
| A6 | ENST00000260896 | RPS24 | 10 | 79138226 | 79145063 |
| A6 | ENSESTT00000021114 | | 10 | 79139712 | 79145062 |
| A6 | ENSESTT00000021113 | | 10 | 79139712 | 79159037 |
| A6 | ENST00000311407 | Q9P1E1 | 10 | 79193716 | 79194732 |
| A6 | ENSESTT00000021115 | | 10 | 79353170 | 79457696 |
| A6 | ENSESTT00000021116 | | 10 | 79371686 | 79382425 |
| A6 | ENSESTT00000021117 | | 10 | 79371802 | 79434851 |
| A6 | ENSESTT00000021118 | | 10 | 79398200 | 79405363 |
| A6 | ENSESTT00000021119 | | 10 | 79800422 | 79801382 |
| A6 | ENSESTT00000021253 | | 10 | 80160384 | 80173420 |
| A6 | ENSESTT00000021230 | | 10 | 80244085 | 80395502 |
| A6 | ENST00000334512 | RAI17 | 10 | 80244114 | 80417109 |
| A6 | ENSESTT00000021231 | | 10 | 80348029 | 80395502 |
| A6 | ENST00000277788 | RAI17 | 10 | 80348029 | 80417109 |
| A6 | ENSESTT00000021232 | | 10 | 80410611 | 80417213 |
| A6 | ENSESTT00000021233 | | 10 | 80451828 | 80458345 |
| A6 | ENST00000225174 | PPIF | 10 | 80451837 | 80459684 |
| A6 | ENSESTT00000021234 | | 10 | 80486687 | 80513633 |
| A6 | ENST00000298180 | NM_153367 | 10 | 80486688 | 80549986 |
| A6 | ENSESTT00000021252 | | 10 | 80498696 | 80549886 |
| A6 | ENSESTT00000021251 | | 10 | 80608432 | 80610640 |
| A6 | ENST00000329262 | | 10 | 80617009 | 80617473 |
| A6 | ENST00000328784 | SFTPA2 | 10 | 80661568 | 80663842 |
| A6 | ENSESTT00000021246 | | 10 | 80661840 | 80664756 |
| A6 | ENSESTT00000021247 | | 10 | 80661841 | 80664756 |
| A6 | ENSESTT00000021248 | | 10 | 80661863 | 80664744 |
| A6 | ENSESTT00000021245 | | 10 | 80663244 | 80984138 |
| A6 | ENST00000334432 | SFTPA1 | 10 | 80715314 | 80718472 |
| A6 | ENST00000329658 | | 10 | 80716284 | 80718472 |
| A6 | ENSESTT00000021235 | | 10 | 80718201 | 81039562 |
| A6 | ENSESTT00000021237 | | 10 | 80718531 | 81039746 |
| A6 | ENSESTT00000021238 | | 10 | 80718764 | 81039731 |
| A6 | ENSESTT00000021241 | | 10 | 80718926 | 80719667 |
| A6 | ENSESTT00000021239 | | 10 | 80719112 | 81039731 |
| A6 | ENSESTT00000021240 | | 10 | 80719228 | 81039731 |
| A6 | ENSESTT00000021236 | | 10 | 80772270 | 81190839 |
| A6 | ENSESTT00000021250 | | 10 | 80789921 | 80795955 |
| A6 | ENST00000241878 | Q9H392 | 10 | 80852848 | 80853054 |
| A6 | ENSESTT00000021249 | | 10 | 80928323 | 80930532 |
| A6 | ENST00000333539 | | 10 | 80936971 | 80937273 |
| A6 | ENST00000242457 | SFTPA2 | 10 | 80981513 | 80983787 |
| A6 | ENSESTT00000021242 | | 10 | 80981785 | 80984701 |
| A6 | ENSESTT00000021243 | | 10 | 80981786 | 80984701 |
| A6 | ENSESTT00000021244 | | 10 | 80981808 | 80984689 |
| A6 | ENSESTT00000021401 | | 10 | 81035257 | 81038146 |
| A6 | ENST00000242455 | SFTPA1 | 10 | 81035260 | 81038418 |
| A6 | ENSESTT00000021402 | | 10 | 81035266 | 81038139 |
| A6 | ENSESTT00000021403 | | 10 | 81035780 | 81036737 |
| A6 | ENSESTT00000021455 | | 10 | 81038872 | 81039613 |
| A6 | ENSESTT00000021404 | | 10 | 81093936 | 81101184 |
| A6 | ENSESTT00000021454 | | 10 | 81110118 | 81116151 |
| A6 | ENSESTT00000021446 | | 10 | 81116552 | 81250849 |
| A6 | ENST00000335456 | | 10 | 81128338 | 81275386 |
| A6 | ENST00000334434 | Q9H392 | 10 | 81173024 | 81173230 |
| A6 | ENSESTT00000021451 | | 10 | 81190668 | 81230459 |
| A6 | ENSESTT00000021447 | | 10 | 81190746 | 81250724 |
| A6 | ENSESTT00000021453 | | 10 | 81202274 | 81202689 |
| A6 | ENSESTT00000021448 | | 10 | 81226317 | 81250724 |
| A6 | ENSESTT00000021450 | | 10 | 81228390 | 81238987 |
| A6 | ENSESTT00000021452 | | 10 | 81228392 | 81230459 |
| A6 | ENSESTT00000021449 | | 10 | 81228392 | 81250724 |
| A6 | ENST00000305740 | | 10 | 81268629 | 81274246 |
| A6 | ENST00000298189 | Q96LH7 | 10 | 81268792 | 81275205 |
| A6 | ENSESTT00000021445 | | 10 | 81294428 | 81295438 |
| A6 | ENSESTT00000021405 | | 10 | 81329237 | 81332141 |
| A6 | ENST00000312535 | | 10 | 81344660 | 81347359 |
| A6 | ENSESTT00000021406 | | 10 | 81346244 | 81347451 |
| A6 | ENSESTT00000021444 | | 10 | 81362073 | 81373418 |
| A6 | ENSESTT00000021443 | | 10 | 81362073 | 81373430 |
| A6 | ENST00000256035 | SFTPD | 10 | 81362085 | 81373438 |
| A6 | ENST00000302577 | | 10 | 81456301 | 81456726 |
| A6 | ENSESTT00000021407 | | 10 | 81503006 | 81516880 |
| A6 | ENSESTT00000021408 | | 10 | 81503009 | 81516880 |
| A6 | ENST00000256052 | NM_025125 | 10 | 81503033 | 81516880 |
| A6 | ENSESTT00000021409 | | 10 | 81503580 | 81516880 |
| A6 | ENSESTT00000021410 | | 10 | 81506036 | 81510774 |
| A6 | ENSESTT00000021411 | | 10 | 81506170 | 81515572 |
| A6 | ENSESTT00000021412 | | 10 | 81506477 | 81516880 |
| A6 | ENSESTT00000021442 | | 10 | 81515319 | 81516192 |
| A6 | ENSESTT00000021413 | | 10 | 81557063 | 81569687 |
| A6 | ENSESTT00000021414 | | 10 | 81566481 | 81568673 |
| A6 | ENSESTT00000021415 | | 10 | 81568379 | 81569370 |
| A6 | ENSESTT00000021416 | | 10 | 81579472 | 81582085 |
| A6 | ENSESTT00000021426 | | 10 | 81579821 | 81582361 |
| A6 | ENSESTT00000021430 | | 10 | 81579821 | 81583460 |
| A6 | ENSESTT00000021429 | | 10 | 81579821 | 81587747 |
| A6 | ENSESTT00000021427 | | 10 | 81579932 | 81590496 |
| A6 | ENST00000265447 | ANXA11 | 10 | 81580186 | 81597194 |
| A6 | ENSESTT00000021428 | | 10 | 81583422 | 81587941 |
| A6 | ENSESTT00000021440 | | 10 | 81591229 | 81629900 |
| A6 | ENSESTT00000021441 | | 10 | 81593529 | 81597204 |
| A6 | ENST00000316258 | | 10 | 81671552 | 81672016 |
| A6 | ENST00000280867 | MAT1A | 10 | 81696158 | 81713756 |
| A6 | ENSESTT00000021439 | | 10 | 81697070 | 81697478 |
| A6 | ENSESTT00000021438 | | 10 | 81697983 | 81699025 |
| A6 | ENSESTT00000021437 | | 10 | 81700853 | 81713692 |
| A6 | ENSESTT00000021435 | | 10 | 81700889 | 81714017 |
| A6 | ENSESTT00000021436 | | 10 | 81705012 | 81714017 |
| A6 | ENSESTT00000021433 | | 10 | 81760462 | 81781086 |
| A6 | ENST00000302526 | NM_138812 | 10 | 81762785 | 81776934 |
| A6 | ENSESTT00000021434 | | 10 | 81762846 | 81781047 |
| A6 | ENSESTT00000021417 | | 10 | 81781106 | 81787306 |
| A6 | ENST00000316132 | NM_032372 | 10 | 81781135 | 81792406 |
| A6 | ENSESTT00000021418 | | 10 | 81787282 | 81791494 |
| A6 | ENSESTT00000021419 | | 10 | 81832162 | 81856971 |
| A6 | ENSESTT00000021420 | | 10 | 81832829 | 81856971 |
| A6 | ENSESTT00000021421 | | 10 | 81832918 | 81856971 |
| A6 | ENSESTT00000021422 | | 10 | 81838176 | 81856971 |
| A6 | ENST00000241895 | NM_032333 | 10 | 81838189 | 81856968 |
| A6 | ENST00000312169 | Q9BU34 | 10 | 81913567 | 81942309 |
| A6 | ENST00000316064 | Q8N2P5 | 10 | 81913567 | 81945181 |
| A6 | ENST00000265450 | NM_030927 | 10 | 81913567 | 81946237 |
| A6 | ENSESTT00000021423 | | 10 | 81913573 | 81947518 |
| A6 | ENSESTT00000021432 | | 10 | 81939416 | 81943437 |
| A6 | ENSESTT00000021424 | | 10 | 81940535 | 81942599 |
| A6 | ENSESTT00000021425 | | 10 | 81940630 | 81956185 |
| A6 | ENSESTT00000021431 | | 10 | 81953930 | 81960275 |
| A6 | ENST00000329171 | | 10 | 81959343 | 81959717 |
| A6 | ENST00000313455 | Q8NB58 | 10 | 82067342 | 82068641 |
| A6 | ENSESTT00000021107 | | 10 | 82074130 | 82078067 |
| A7 | OTTHUMT00007008148 | Hs_7_c5082 | 7 | 71452603 | 71709685 |
| A7 | ENSESTT00000039973 | | 7 | 71571787 | 71621457 |
| A7 | OTTHUMT00007007334 | Hs_7_c1064 | 7 | 71599963 | 71600172 |
| A7 | ENST00000265301 | | 7 | 71605907 | 71606016 |
| A7 | OTTHUMT00007007336 | Hs_7_c1066 | 7 | 71645929 | 71646324 |
| A7 | ENSESTT00000039972 | | 7 | 71654577 | 71698179 |
| A7 | ENSESTT00000039971 | | 7 | 71689895 | 71710894 |
| A7 | ENSESTT00000039970 | | 7 | 71709544 | 71710900 |
| A7 | ENSESTT00000039903 | | 7 | 71712159 | 71716707 |
| A7 | ENSESTT00000039904 | | 7 | 71712178 | 71714460 |
| A7 | OTTHUMT00007006933 | LOC155370 | 7 | 71712203 | 71716703 |
| A7 | ENSESTT00000039905 | | 7 | 71712367 | 71716710 |
| A7 | OTTHUMT00007007337 | Hs_7_c1068 | 7 | 71746587 | 71753122 |
| A7 | ENSESTT00000039948 | | 7 | 71746639 | 71904642 |
| A7 | OTTHUMT00007007338 | Hs_7_c1069 | 7 | 71747922 | 71748002 |
| A7 | ENST00000323915 | | 7 | 71749050 | 71751686 |
| A7 | ENSESTT00000039906 | | 7 | 71762023 | 71773321 |
| A7 | ENSESTT00000039911 | | 7 | 71762045 | 71763010 |
| A7 | ENSESTT00000039910 | | 7 | 71762045 | 71822067 |
| A7 | ENSESTT00000039909 | | 7 | 71762045 | 71822522 |
| A7 | ENSESTT00000039908 | | 7 | 71762045 | 72120515 |
| A7 | ENSESTT00000039907 | | 7 | 71762045 | 72121873 |
| A7 | OTTHUMT00007006851 | POM121 | 7 | 71762053 | 71834096 |
| A7 | ENST00000275580 | O95746 | 7 | 71762053 | 71922346 |
| A7 | ENSESTT00000039912 | | 7 | 71773689 | 71796697 |
| A7 | ENST00000257622 | POM121 | 7 | 71809068 | 71828890 |
| A7 | ENSESTT00000039913 | | 7 | 71823101 | 72121996 |
| A7 | ENSESTT00000039914 | | 7 | 71823135 | 71830954 |
| A7 | ENSESTT00000039938 | | 7 | 71830947 | 71831469 |
| A7 | ENSESTT00000039937 | | 7 | 71830947 | 71831838 |
| A7 | ENSESTT00000039935 | | 7 | 71830947 | 71831880 |
| A7 | ENSESTT00000039933 | | 7 | 71830947 | 71831893 |
| A7 | ENSESTT00000039931 | | 7 | 71830947 | 71833202 |
| A7 | ENSESTT00000039929 | | 7 | 71830947 | 71837351 |
| A7 | ENSESTT00000039926 | | 7 | 71830947 | 71837371 |
| A7 | ENSESTT00000039925 | | 7 | 71830947 | 71837390 |
| A7 | ENSESTT00000039936 | | 7 | 71830954 | 71831875 |
| A7 | ENSESTT00000039934 | | 7 | 71830954 | 71831893 |
| A7 | ENSESTT00000039932 | | 7 | 71830954 | 71833202 |
| A7 | ENSESTT00000039930 | | 7 | 71830954 | 71837351 |
| A7 | ENSESTT00000039927 | | 7 | 71830954 | 71837371 |
| A7 | OTTHUMT00007007028 | WBSCR20A.1 | 7 | 71830955 | 71837376 |
| A7 | ENST00000330999 | NM_148936 | 7 | 71831040 | 71832800 |
| A7 | ENST00000222857 | NM_149379 | 7 | 71831040 | 71834307 |
| A7 | ENSESTT00000039923 | | 7 | 71831952 | 72131287 |
| A7 | ENSESTT00000039928 | | 7 | 71832127 | 71837371 |
| A7 | ENSESTT00000039924 | | 7 | 71836013 | 71837392 |
| A7 | ENST00000285805 | Q86UV6 | 7 | 71842133 | 71852114 |
| A7 | ENSESTT00000039939 | | 7 | 71842201 | 71849602 |
| A7 | OTTHUMT00007006839 | mfhmh_h_70400969_71300968_m_133605736_13 | 7 | 71842530 | 71848805 |
| A7 | ENSESTT00000039940 | | 7 | 71848707 | 72154649 |
| A7 | ENSESTT00000039915 | | 7 | 71851502 | 71854934 |
| A7 | ENST00000335315 | Q8N4N6 | 7 | 71852127 | 71852366 |
| A7 | OTTHUMT00007007357 | Hs_7_c1076 | 7 | 71852462 | 71854375 |
| A7 | OTTHUMT00007007220 | DKFZP434A0131.3 | 7 | 71857195 | 71882155 |
| A7 | ENST00000305954 | NM_018991 | 7 | 71857213 | 71882155 |
| A7 | ENSESTT00000039969 | | 7 | 71876851 | 71877435 |
| A7 | ENSESTT00000039916 | | 7 | 71877080 | 72107101 |
| A7 | ENSESTT00000039962 | | 7 | 71880796 | 71888562 |
| A7 | ENSESTT00000039963 | | 7 | 71880796 | 71888562 |
| A7 | ENSESTT00000039949 | | 7 | 71880796 | 71888584 |
| A7 | ENSESTT00000039950 | | 7 | 71880796 | 71888584 |
| A7 | ENSESTT00000039964 | | 7 | 71881128 | 71888562 |
| A7 | ENSESTT00000039965 | | 7 | 71881128 | 71888562 |
| A7 | ENSESTT00000039951 | | 7 | 71881128 | 71888584 |
| A7 | ENSESTT00000039952 | | 7 | 71881128 | 71888584 |
| A7 | ENSESTT00000039953 | | 7 | 71881128 | 71888584 |
| A7 | ENSESTT00000039966 | | 7 | 71881144 | 71888562 |
| A7 | ENSESTT00000039954 | | 7 | 71881144 | 71888584 |
| A7 | ENSESTT00000039955 | | 7 | 71881144 | 71888584 |
| A7 | ENSESTT00000039956 | | 7 | 71881144 | 71888584 |
| A7 | ENSESTT00000039957 | | 7 | 71881144 | 71888584 |
| A7 | ENSESTT00000039967 | | 7 | 71881902 | 71888562 |
| A7 | ENSESTT00000039958 | | 7 | 71881902 | 71888584 |
| A7 | ENSESTT00000039959 | | 7 | 71881902 | 71888584 |
| A7 | ENSESTT00000039960 | | 7 | 71882400 | 71888584 |
| A7 | ENSESTT00000039961 | | 7 | 71882400 | 71888584 |
| A7 | ENSESTT00000039968 | | 7 | 71882996 | 71888562 |
| A7 | OTTHUMT00007007358 | Hs_7_c1079 | 7 | 71888246 | 71931788 |
| A7 | ENSESTT00000039917 | | 7 | 71888736 | 71922142 |
| A7 | ENSESTT00000039918 | | 7 | 71888768 | 71932926 |
| A7 | ENSESTT00000039920 | | 7 | 71888780 | 71904881 |
| A7 | ENSESTT00000039919 | | 7 | 71888780 | 71932926 |
| A7 | OTTHUMT00007006363 | mbhmh_gw1304124.71119255.71153128.3.2e−3 | 7 | 71892093 | 71895794 |
| A7 | ENSESTT00000039921 | | 7 | 71894107 | 71932926 |
| A7 | ENST00000334824 | Q86WY7 | 7 | 71902561 | 71908486 |
| A7 | ENSESTT00000039944 | | 7 | 71903628 | 71905036 |
| A7 | ENSESTT00000039943 | | 7 | 71904798 | 71932687 |
| A7 | ENSESTT00000039947 | | 7 | 71908198 | 71909384 |
| A7 | ENSESTT00000039946 | | 7 | 71910560 | 71912409 |
| A7 | ENSESTT00000039945 | | 7 | 71918103 | 71919942 |
| A7 | ENST00000323689 | | 7 | 71920032 | 71931791 |
| A7 | ENST00000306533 | PMS2L5 | 7 | 71922114 | 71931791 |
| A7 | ENST00000335506 | Q86WY7 | 7 | 71930603 | 71936531 |
| A7 | ENSESTT00000039942 | | 7 | 71931673 | 71933081 |
| A7 | OTTHUMT00007006797 | mfhmh_H_NH0396K03_F218045.fgenesh2.5 | 7 | 71931784 | 71940793 |
| A7 | ENSESTT00000039941 | | 7 | 71936243 | 71937429 |
| A7 | ENSESTT00000039922 | | 7 | 71981407 | 72009414 |
| A7 | OTTHUMT00007007381 | Hs_7_c1083 | 7 | 72001548 | 72031575 |
| A7 | ENSESTT00000041207 | | 7 | 72001575 | 72012822 |
| A7 | ENSESTT00000041210 | | 7 | 72016228 | 72028278 |
| A7 | ENSESTT00000041209 | | 7 | 72016228 | 72031964 |
| A7 | ENSESTT00000041208 | | 7 | 72016228 | 72032675 |
| A7 | ENSESTT00000041225 | | 7 | 72016228 | 72032723 |
| A7 | ENSESTT00000041224 | | 7 | 72016228 | 72032724 |
| A7 | ENSESTT00000041215 | | 7 | 72017507 | 72023278 |
| A7 | ENSESTT00000041214 | | 7 | 72017507 | 72031964 |
| A7 | ENSESTT00000041213 | | 7 | 72017507 | 72032675 |
| A7 | ENSESTT00000041212 | | 7 | 72017507 | 72032723 |
| A7 | ENSESTT00000041211 | | 7 | 72017507 | 72032724 |
| A7 | ENSESTT00000041220 | | 7 | 72018632 | 72028278 |
| A7 | ENSESTT00000041219 | | 7 | 72018632 | 72031964 |
| A7 | ENSESTT00000041218 | | 7 | 72018632 | 72032675 |
| A7 | ENSESTT00000041217 | | 7 | 72018632 | 72032723 |
| A7 | ENSESTT00000041216 | | 7 | 72018632 | 72032724 |
| A7 | ENSESTT00000041221 | | 7 | 72020761 | 72028278 |
| A7 | ENSESTT00000041222 | | 7 | 72028242 | 72032724 |
| A7 | ENSESTT00000041223 | | 7 | 72029512 | 72032724 |
| A7 | ENSESTT00000041228 | | 7 | 72046727 | 72056376 |
| A7 | ENSESTT00000041226 | | 7 | 72046727 | 72062034 |
| A7 | ENSESTT00000041227 | | 7 | 72046727 | 72062034 |
| A7 | OTTHUMT00007006731 | NCF1.1 | 7 | 72046768 | 72062076 |
| A7 | ENSESTT00000041229 | | 7 | 72046771 | 72056068 |
| A7 | ENST00000330925 | | 7 | 72046778 | 72061921 |
| A7 | ENSESTT00000041230 | | 7 | 72057999 | 72062076 |
| A7 | ENSESTT00000041231 | | 7 | 72058029 | 72062076 |
| A7 | ENST00000297906 | | 7 | 72069401 | 72070888 |
| A7 | ENSESTT00000041281 | | 7 | 72077500 | 72079722 |
| A7 | OTTHUMT00007008151 | Hs_7_c5085 | 7 | 72085452 | 72106243 |
| A7 | OTTHUMT00007006556 | mbhmh_h_71200968_72100967_m_133605736_13 | 7 | 72111678 | 72127258 |
| A7 | ENST00000308082 | | 7 | 72119594 | 72129318 |
| A7 | OTTHUMT00007007030 | WBSCR20A.2 | 7 | 72129323 | 72134901 |
| A7 | ENST00000310326 | WBSCR20A | 7 | 72129324 | 72134920 |
| A7 | ENSESTT00000041279 | | 7 | 72129920 | 72134917 |
| A7 | ENSESTT00000041278 | | 7 | 72130393 | 72133598 |
| A7 | ENSESTT00000041280 | | 7 | 72130841 | 72134862 |
| A7 | ENST00000333149 | NM_178125 | 7 | 72138632 | 72154182 |
| A7 | OTTHUMT00007007384 | Hs_7_c1089 | 7 | 72139056 | 72150837 |
| A7 | ENSESTT00000041277 | | 7 | 72139434 | 72143038 |
| A7 | ENSESTT00000041276 | | 7 | 72139434 | 72144953 |
| A7 | ENSESTT00000041275 | | 7 | 72139434 | 72151653 |
| A7 | OTTHUMT00007007413 | Hs_7_c1090 | 7 | 72140207 | 72141310 |
| A7 | ENST00000252037 | FKBP6 | 7 | 72154529 | 72168994 |
| A7 | OTTHUMT00007006557 | mbhmh_h_71200968_72100967_m_133605736_13 | 7 | 72154529 | 72168994 |
| A7 | ENSESTT00000041233 | | 7 | 72156394 | 72184728 |
| A7 | ENSESTT00000041232 | | 7 | 72156394 | 72184733 |
| A7 | ENSESTT00000041234 | | 7 | 72157790 | 72168998 |
| A7 | OTTHUMT00007006375 | FZD9 | 7 | 72260410 | 72262589 |
| A7 | ENST00000265756 | BAZ1B | 7 | 72266830 | 72348646 |
| A7 | OTTHUMT00007007171 | BAZ1B | 7 | 72266830 | 72348712 |
| A7 | ENSESTT00000041274 | | 7 | 72268726 | 72273720 |
| A7 | ENSESTT00000041273 | | 7 | 72277272 | 72303421 |
| A7 | ENSESTT00000041272 | | 7 | 72304601 | 72348524 |
| A7 | ENSESTT00000041271 | | 7 | 72304601 | 72348733 |
| A7 | ENSESTT00000041266 | | 7 | 72362783 | 72383988 |
| A7 | ENST00000223368 | BCL7B | 7 | 72362783 | 72384121 |
| A7 | OTTHUMT00007006736 | BCL7B | 7 | 72362783 | 72384121 |
| A7 | ENSESTT00000041269 | | 7 | 72363456 | 72383648 |
| A7 | ENSESTT00000041264 | | 7 | 72363456 | 72384093 |
| A7 | ENSESTT00000041263 | | 7 | 72363469 | 72384120 |
| A7 | ENSESTT00000041267 | | 7 | 72363476 | 72383988 |
| A7 | ENSESTT00000041270 | | 7 | 72363528 | 72383646 |
| A7 | ENSESTT00000041268 | | 7 | 72363620 | 72383668 |
| A7 | ENSESTT00000041265 | | 7 | 72363620 | 72384016 |
| A7 | ENSESTT00000041262 | | 7 | 72363722 | 72384156 |
| A7 | OTTHUMT00007006751 | TBL2 | 7 | 72396096 | 72405069 |
| A7 | ENST00000275621 | TBL2 | 7 | 72396099 | 72405018 |
| A7 | ENSESTT00000041260 | | 7 | 72396857 | 72405034 |
| A7 | ENSESTT00000041261 | | 7 | 72396857 | 72405034 |
| A7 | ENST00000305632 | NM_032988 | 7 | 72403031 | 72404976 |
| A7 | ENST00000243720 | WBSCR14 | 7 | 72419621 | 72450967 |
| A7 | OTTHUMT00007006613 | WBSCR14 | 7 | 72419621 | 72450967 |
| A7 | ENSESTT00000041259 | | 7 | 72420796 | 72422929 |
| A7 | ENST00000313375 | WBSCR14 | 7 | 72422850 | 72450919 |
| A7 | ENSESTT00000041257 | | 7 | 72424072 | 72450970 |
| A7 | ENSESTT00000041258 | | 7 | 72433738 | 72450946 |
| A7 | OTTHUMT00007007252 | mbhmh_h_71200968_72100967_m_133605736_13 | 7 | 72478242 | 72507686 |
| A7 | ENST00000324941 | WBSCR24 | 7 | 72494272 | 72497803 |
| A7 | ENSESTT00000041235 | | 7 | 72494282 | 72497608 |
| A7 | OTTHUMT00007006614 | WBSCR18 | 7 | 72508698 | 72509869 |
| A7 | ENST00000324842 | WBSCR18 | 7 | 72509170 | 72509850 |
| A7 | ENSESTT00000041236 | | 7 | 72510028 | 72524583 |
| A7 | ENST00000265758 | WBSCR22 | 7 | 72510034 | 72524313 |
| A7 | OTTHUMT00007006630 | WBSCR22 | 7 | 72510034 | 72524639 |
| A7 | ENSESTT00000041237 | | 7 | 72510055 | 72524583 |
| A7 | ENSESTT00000041238 | | 7 | 72510072 | 72524583 |
| A7 | ENSESTT00000041239 | | 7 | 72510232 | 72519061 |
| A7 | ENSESTT00000041240 | | 7 | 72517339 | 72524579 |
| A7 | ENSESTT00000041241 | | 7 | 72519822 | 72524424 |
| A7 | ENSESTT00000041242 | | 7 | 72520410 | 72524569 |
| A7 | ENST00000222812 | STX1A | 7 | 72525637 | 72546059 |
| A7 | OTTHUMT00007006498 | STX1A | 7 | 72525637 | 72546086 |
| A7 | ENSESTT00000041256 | | 7 | 72526715 | 72530923 |
| A7 | ENSESTT00000041252 | | 7 | 72526715 | 72546118 |
| A7 | ENSESTT00000041253 | | 7 | 72526718 | 72546118 |
| A7 | ENSESTT00000041254 | | 7 | 72530608 | 72546099 |
| A7 | ENSESTT00000041255 | | 7 | 72531576 | 72546069 |
| A7 | OTTHUMT00007006628 | WBSCR21 | 7 | 72562521 | 72565258 |
| A7 | ENST00000322862 | NM_148914 | 7 | 72562544 | 72565281 |
| A7 | ENST00000222800 | WBSCR21 | 7 | 72562986 | 72565224 |
| A7 | OTTHUMT00007006748 | CLDN3 | 7 | 72595424 | 72596673 |
| A7 | ENST00000297926 | CLDN3 | 7 | 72595814 | 72596476 |
| A7 | OTTHUMT00007006403 | nh_nm_gi18182840 | 7 | 72655639 | 72656526 |
| A7 | OTTHUMT00007006749 | CLDN4 | 7 | 72657433 | 72659112 |
| A7 | ENSESTT00000041251 | | 7 | 72661015 | 72666499 |
| A7 | ENST00000297873 | NM_152559 | 7 | 72661023 | 72668960 |
| A7 | OTTHUMT00007007226 | mbhmh_nh_h_71200968_72100967_m_132805737 | 7 | 72661138 | 72668567 |
| A7 | ENSESTT00000041243 | | 7 | 72687586 | 72692202 |
| A7 | ENST00000320531 | NM_182504 | 7 | 72687586 | 72692314 |
| A7 | OTTHUMT00007007227 | mbhmh_nh_h_71200968_72100967_m_132805737 | 7 | 72691651 | 72707065 |
| A7 | ENSESTT00000041244 | | 7 | 72854539 | 72869441 |
| A7 | ENST00000320425 | ELN | 7 | 72854615 | 72895127 |
| A7 | ENST00000309678 | Q14235 | 7 | 72854615 | 72895127 |
| A7 | ENST00000320492 | Q8NBI4 | 7 | 72854615 | 72895127 |
| A7 | OTTHUMT00007006288 | ELN | 7 | 72854615 | 72895127 |
| A7 | ENST00000320399 | Q8N2G0 | 7 | 72854615 | 72896103 |
| A7 | ENST00000252034 | O15337 | 7 | 72861793 | 72887565 |
| A7 | ENSESTT00000041245 | | 7 | 72879278 | 72882774 |
| A7 | ENSESTT00000041246 | | 7 | 72883099 | 72889621 |
| A7 | ENSESTT00000041247 | | 7 | 72892119 | 72895391 |
| A7 | ENST00000265761 | LIMK1 | 7 | 72910253 | 72948951 |
| A7 | OTTHUMT00007006772 | LIMK1 | 7 | 72910253 | 72948951 |
| A7 | ENSESTT00000041248 | | 7 | 72919602 | 72925577 |
| A7 | ENSESTT00000041249 | | 7 | 72932555 | 72947826 |
| A7 | ENSESTT00000041250 | | 7 | 72938074 | 72947826 |
| A7 | ENSESTT00000036020 | | 7 | 73000780 | 73021942 |
| A7 | ENSESTT00000036019 | | 7 | 73000780 | 73023522 |
| A7 | ENSESTT00000036021 | | 7 | 73000783 | 73016740 |
| A7 | ENSESTT00000036016 | | 7 | 73000792 | 73021897 |
| A7 | ENSESTT00000036017 | | 7 | 73000792 | 73021897 |
| A7 | ENST00000265754 | WBSCR1 | 7 | 73000803 | 73023526 |
| A7 | OTTHUMT00007006349 | WBSCR1 | 7 | 73000803 | 73023526 |
| A7 | ENST00000265753 | WBSCR1 | 7 | 73000811 | 73021735 |
| A7 | ENSESTT00000036018 | | 7 | 73000834 | 73023522 |
| A7 | ENSESTT00000036027 | | 7 | 73036361 | 73046683 |
| A7 | ENST00000309368 | WBSCR5 | 7 | 73036372 | 73056254 |
| A7 | OTTHUMT00007006353 | WBSCR5 | 7 | 73036372 | 73056260 |
| A7 | ENSESTT00000036028 | | 7 | 73036409 | 73047047 |
| A7 | ENSESTT00000036029 | | 7 | 73036413 | 73055741 |
| A7 | ENSESTT00000036030 | | 7 | 73036479 | 73055741 |
| A7 | ENSESTT00000036031 | | 7 | 73036487 | 73056261 |
| A7 | ENSESTT00000036033 | | 7 | 73041247 | 73055741 |
| A7 | ENSESTT00000036032 | | 7 | 73041247 | 73056261 |
| A7 | ENSESTT00000036034 | | 7 | 73042370 | 73055741 |
| A7 | ENST00000315652 | WBSCR5 | 7 | 73042403 | 73051169 |
| A7 | ENSESTT00000036035 | | 7 | 73046885 | 73055741 |
| A7 | ENST00000055077 | RFC2 | 7 | 73057931 | 73080835 |
| A7 | OTTHUMT00007006563 | RFC2 | 7 | 73058100 | 73080829 |
| A7 | ENSESTT00000036108 | | 7 | 73058332 | 73076209 |
| A7 | ENSESTT00000036107 | | 7 | 73058332 | 73080717 |
| A7 | ENSESTT00000036106 | | 7 | 73058332 | 73080816 |
| A7 | ENSESTT00000036105 | | 7 | 73058332 | 73080827 |
| A7 | ENSESTT00000036103 | | 7 | 73058332 | 73080835 |
| A7 | ENST00000275627 | RFC2 | 7 | 73058533 | 73080810 |
| A7 | ENSESTT00000036104 | | 7 | 73058566 | 73080828 |
| A7 | OTTHUMT00007006304 | CYLN2 | 7 | 73115902 | 73232362 |
| A7 | ENST00000275634 | CYLN2 | 7 | 73143935 | 73227994 |
| A7 | ENST00000223398 | CYLN2 | 7 | 73143974 | 73227994 |
| A7 | ENSESTT00000036039 | | 7 | 73183000 | 73202399 |
| A7 | ENSESTT00000036040 | | 7 | 73183883 | 73202625 |
| A7 | ENSESTT00000036041 | | 7 | 73202736 | 73227995 |
| A7 | ENSESTT00000036042 | | 7 | 73203042 | 73227995 |
| A7 | ENSESTT00000036043 | | 7 | 73212952 | 73230806 |
| A7 | OTTHUMT00007007106 | GTF2IRD1 | 7 | 73280217 | 73429013 |
| A7 | ENST00000265755 | GTF2IRD1 | 7 | 73334508 | 73428857 |
| A7 | ENSESTT00000036044 | | 7 | 73346006 | 73381903 |
| A7 | ENSESTT00000036045 | | 7 | 73350521 | 73364601 |
| A7 | OTTHUMT00007006631 | WBSCR23 | 7 | 73358718 | 73361335 |
| A7 | OTTHUMT00007007275 | Hs_7_c1118 | 7 | 73360975 | 73361085 |
| A7 | ENSESTT00000036046 | | 7 | 73373585 | 73429028 |
| A7 | ENSESTT00000036047 | | 7 | 73427384 | 73429022 |
| A7 | ENSESTT00000036048 | | 7 | 73484123 | 73517534 |
| A7 | OTTHUMT00007006793 | GTF2I | 7 | 73484127 | 73587111 |
| A7 | ENSESTT00000036102 | | 7 | 73515362 | 73526731 |
| A7 | ENSESTT00000036051 | | 7 | 73515542 | 73564552 |
| A7 | ENSESTT00000036052 | | 7 | 73515542 | 73564552 |
| A7 | ENSESTT00000036049 | | 7 | 73515542 | 73566495 |
| A7 | ENSESTT00000036050 | | 7 | 73515542 | 73566495 |
| A7 | ENST00000324924 | NM_033000 | 7 | 73515552 | 73585257 |
| A7 | ENST00000324906 | GTF2I | 7 | 73515552 | 73585257 |
| A7 | ENST00000324896 | GTF2I | 7 | 73515552 | 73587109 |
| A7 | ENSESTT00000036053 | | 7 | 73517393 | 73555329 |
| A7 | ENSESTT00000036063 | | 7 | 73569898 | 73575881 |
| A7 | ENSESTT00000036062 | | 7 | 73569898 | 73581959 |
| A7 | ENSESTT00000036061 | | 7 | 73569898 | 73585643 |
| A7 | ENSESTT00000036060 | | 7 | 73569898 | 73586353 |
| A7 | ENSESTT00000036059 | | 7 | 73569898 | 73586387 |
| A7 | ENSESTT00000036058 | | 7 | 73569898 | 73586401 |
| A7 | ENSESTT00000036069 | | 7 | 73571185 | 73575881 |
| A7 | ENSESTT00000036068 | | 7 | 73571185 | 73581959 |
| A7 | ENSESTT00000036067 | | 7 | 73571185 | 73585643 |
| A7 | ENSESTT00000036066 | | 7 | 73571185 | 73586353 |
| A7 | ENSESTT00000036065 | | 7 | 73571185 | 73586387 |
| A7 | ENSESTT00000036064 | | 7 | 73571185 | 73586401 |
| A7 | ENSESTT00000036074 | | 7 | 73572309 | 73581959 |
| A7 | ENSESTT00000036073 | | 7 | 73572309 | 73585643 |
| A7 | ENSESTT00000036072 | | 7 | 73572309 | 73586353 |
| A7 | ENSESTT00000036071 | | 7 | 73572309 | 73586387 |
| A7 | ENSESTT00000036070 | | 7 | 73572309 | 73586401 |
| A7 | ENSESTT00000036075 | | 7 | 73574439 | 73581959 |
| A7 | ENSESTT00000036076 | | 7 | 73583165 | 73585027 |
| A7 | ENSESTT00000036077 | | 7 | 73584379 | 73586387 |
| A7 | OTTHUMT00007006732 | NCF1.2 | 7 | 73600398 | 73615746 |
| A7 | ENSESTT00000036086 | | 7 | 73600417 | 73610049 |
| A7 | ENST00000289473 | NCF1 | 7 | 73600468 | 73615593 |
| A7 | ENSESTT00000036087 | | 7 | 73611674 | 73615748 |
| A7 | ENSESTT00000036088 | | 7 | 73611704 | 73615748 |
| A7 | ENSESTT00000036089 | | 7 | 73614408 | 73615753 |
| A7 | ENST00000302215 | NM_032203 | 7 | 73622573 | 73679914 |
| A7 | OTTHUMT00007006446 | mbhmh_H_NH0813J07_F171046.fgenesh2.2 | 7 | 73623090 | 73663589 |
| A7 | ENSESTT00000036101 | | 7 | 73659841 | 73679927 |
| A7 | OTTHUMT00007007215 | DKFZP434A0131.1 | 7 | 73706764 | 73718764 |
| A7 | ENST00000308103 | NM_018991 | 7 | 73710352 | 73713295 |
| A7 | OTTHUMT00007008153 | Hs_7_c5086 | 7 | 73711129 | 73713351 |
| A7 | OTTHUMT00007007303 | Hs_7_c1127 | 7 | 73718511 | 73733865 |
| A7 | ENST00000318547 | PMS2L5 | 7 | 73718985 | 73733868 |
| A7 | ENSESTT00000036090 | | 7 | 73718990 | 73726032 |
| A7 | ENSESTT00000036091 | | 7 | 73719013 | 73733949 |
| A7 | ENST00000318568 | Q16673 | 7 | 73722332 | 73729180 |
| A7 | ENSESTT00000036092 | | 7 | 73726925 | 73728321 |
| A7 | ENST00000333385 | Q86WY7 | 7 | 73732679 | 73739325 |
| A7 | OTTHUMT00007006291 | mfhmh_chr7.73.013.a | 7 | 73733861 | 73770943 |
| A7 | ENST00000328350 | | 7 | 73737708 | 73740340 |
| A7 | ENST00000330313 | | 7 | 73791140 | 73844306 |
| A7 | ENST00000332301 | | 7 | 73791172 | 73850892 |
| A7 | ENSESTT00000036097 | | 7 | 73868813 | 73883752 |
| A7 | ENST00000329959 | WBSCR16 | 7 | 73869229 | 73901788 |
| A7 | ENSESTT00000036098 | | 7 | 73882190 | 73901764 |
| A7 | ENSESTT00000036099 | | 7 | 73889107 | 73901764 |
| A7 | ENSESTT00000036100 | | 7 | 73892480 | 73901764 |
| A7 | ENST00000334260 | Q86WX4 | 7 | 73920467 | 73954376 |
| A7 | ENSESTT00000036093 | | 7 | 73920470 | 73940594 |
| A7 | ENST00000312575 | NM_032203 | 7 | 73962893 | 73977192 |
| A7 | ENSESTT00000036094 | | 7 | 73966809 | 73969279 |
| A7 | ENST00000297905 | | 7 | 73984689 | 73999836 |
| A7 | ENSESTT00000036095 | | 7 | 73990260 | 73999885 |
| A7 | ENSESTT00000036096 | | 7 | 73990542 | 73999843 |
| A7 | ENSESTT00000037870 | | 7 | 74013902 | 74027990 |
| A7 | ENSESTT00000037865 | | 7 | 74013902 | 74029114 |
| A7 | ENSESTT00000037876 | | 7 | 74013902 | 74030393 |
| A7 | ENSESTT00000037871 | | 7 | 74013903 | 74027990 |
| A7 | ENSESTT00000037866 | | 7 | 74013903 | 74029114 |
| A7 | ENSESTT00000037877 | | 7 | 74013903 | 74030393 |
| A7 | ENSESTT00000037872 | | 7 | 74013951 | 74027990 |
| A7 | ENSESTT00000037867 | | 7 | 74013951 | 74029114 |
| A7 | ENSESTT00000037878 | | 7 | 74013951 | 74030393 |
| A7 | ENSESTT00000037873 | | 7 | 74014662 | 74027990 |
| A7 | ENSESTT00000037868 | | 7 | 74014662 | 74029114 |
| A7 | ENSESTT00000037879 | | 7 | 74014662 | 74030393 |
| A7 | ENSESTT00000037875 | | 7 | 74018346 | 74025862 |
| A7 | ENSESTT00000037874 | | 7 | 74018346 | 74027990 |
| A7 | ENSESTT00000037869 | | 7 | 74018346 | 74029114 |
| A7 | ENSESTT00000037880 | | 7 | 74018346 | 74030393 |
| A7 | ENSESTT00000037864 | | 7 | 74033799 | 74065179 |
| A7 | ENST00000335657 | Q86WY7 | 7 | 74111817 | 74114021 |
| A7 | ENSESTT00000037828 | | 7 | 74114121 | 74528229 |
| A7 | ENSESTT00000037860 | | 7 | 74114936 | 74600365 |
| A7 | ENST00000329909 | PMS2L5 | 7 | 74115017 | 74124654 |
| A7 | ENST00000311576 | | 7 | 74115017 | 74126741 |
| A7 | ENSESTT00000037831 | | 7 | 74378046 | 74402224 |
| A7 | ENSESTT00000037829 | | 7 | 74386841 | 74402224 |
| A7 | ENSESTT00000037830 | | 7 | 74400341 | 74402224 |
| A7 | ENSESTT00000037832 | | 7 | 74412873 | 74413761 |
| A7 | ENSESTT00000037833 | | 7 | 74415176 | 74417728 |
| A7 | ENST00000317042 | Q9P1E6 | 7 | 74415789 | 74416214 |
| A7 | OTTHUMT00007006787 | mbhmh_h_72800966_73394042_m_132005738_13 | 7 | 74418388 | 74470222 |
| A7 | ENST00000311251 | | 7 | 74419694 | 74479430 |
| A7 | OTTHUMT00007007356 | Hs_7_c1145 | 7 | 74523575 | 74584324 |
| A7 | ENST00000333996 | Q86WY7 | 7 | 74524542 | 74556308 |
| A7 | ENST00000335396 | Q86WY7 | 7 | 74526082 | 74528285 |
| A7 | ENST00000302439 | PMS2L6 | 7 | 74526852 | 74541042 |
| A7 | ENSESTT00000037834 | | 7 | 74528385 | 74556252 |
| A7 | ENSESTT00000037862 | | 7 | 74529200 | 74566962 |
| A7 | ENST00000311139 | PMS2L5 | 7 | 74529281 | 74538934 |
| A7 | ENST00000314850 | | 7 | 74529281 | 74541015 |
| A7 | OTTHUMT00007008169 | Hs_7_c5090 | 7 | 74529284 | 74600754 |
| A7 | ENSESTT00000037861 | | 7 | 74538952 | 74567012 |
| A7 | ENST00000251624 | PMS2L6 | 7 | 74554875 | 74569067 |
| A7 | ENSESTT00000037835 | | 7 | 74556408 | 74584271 |
| A7 | ENST00000305928 | | 7 | 74557304 | 74569040 |
| A7 | ENST00000335010 | Q86WY7 | 7 | 74582125 | 74584327 |
| A7 | ENSESTT00000037836 | | 7 | 74584427 | 74743294 |
| A7 | ENSESTT00000037863 | | 7 | 74585242 | 74594942 |
| A7 | ENST00000310939 | | 7 | 74585323 | 74600345 |
| A7 | ENSESTT00000037837 | | 7 | 74600539 | 74603386 |
| A7 | ENSESTT00000037838 | | 7 | 74600635 | 74607984 |
| A7 | OTTHUMT00007008170 | Hs_7_c5091 | 7 | 74605989 | 74608444 |
| A7 | ENST00000325462 | NM_018991 | 7 | 74606045 | 74609003 |
| A7 | OTTHUMT00007007218 | DKFZP434A0131.2 | 7 | 74606957 | 74631908 |
| A7 | ENSESTT00000037840 | | 7 | 74607712 | 74637304 |
| A7 | ENSESTT00000037839 | | 7 | 74607712 | 74640405 |
| A7 | ENSESTT00000037841 | | 7 | 74611418 | 74612259 |
| A7 | ENST00000275590 | Q8WW08 | 7 | 74633749 | 74636644 |
| A7 | ENSESTT00000037859 | | 7 | 74634716 | 74636774 |
| A7 | OTTHUMT00007006277 | mbhmh_h_73557902_74457901_m_133948896_13 | 7 | 74634728 | 74636644 |
| A7 | ENST00000323819 | Q86UV7 | 7 | 74636992 | 74646977 |
| A7 | OTTHUMT00007006661 | mfhmh_h_73557902_74457901_m_133948896_13 | 7 | 74640307 | 74649026 |
| A7 | ENSESTT00000037842 | | 7 | 74640495 | 74645112 |
| A7 | ENSESTT00000037843 | | 7 | 74651721 | 74653100 |
| A7 | OTTHUMT00007007377 | Hs_7_c1152 | 7 | 74651740 | 74657781 |
| A7 | ENSESTT00000037844 | | 7 | 74651742 | 74656591 |
| A7 | ENSESTT00000037845 | | 7 | 74652026 | 74657514 |
| A7 | ENST00000323788 | NM_145645 | 7 | 74654800 | 74657968 |
| A7 | ENSESTT00000037846 | | 7 | 74655026 | 74657893 |
| A7 | ENSESTT00000037847 | | 7 | 74656958 | 74658035 |
| A7 | ENSESTT00000037848 | | 7 | 74657213 | 74657787 |
| A7 | ENST00000257665 | POM121 | 7 | 74658837 | 74684082 |
| A7 | ENSESTT00000037858 | | 7 | 74659912 | 74663188 |
| A7 | OTTHUMT00007006280 | mbhmh_h_73557902_74457901_m_133948896_13 | 7 | 74660168 | 74684082 |
| A7 | ENSESTT00000037857 | | 7 | 74664350 | 74666568 |
| A7 | ENSESTT00000037856 | | 7 | 74666904 | 74679035 |
| A7 | ENSESTT00000037855 | | 7 | 74682449 | 74716115 |
| A7 | ENSESTT00000037854 | | 7 | 74716469 | 74769374 |
| A7 | ENST00000301990 | | 7 | 74736524 | 74745533 |
| A7 | OTTHUMT00007007168 | mbhmh_ts.74.012.a | 7 | 74738139 | 74744350 |
| A7 | OTTHUMT00007007383 | Hs_7_c1157 | 7 | 74744346 | 74752527 |
| A7 | ENST00000248606 | PMS2L3 | 7 | 74749246 | 74756200 |
| A7 | ENST00000301956 | PMS2L9 | 7 | 74752342 | 74769228 |
| A7 | OTTHUMT00007007380 | Hs_7_c1154 | 7 | 74753806 | 74769228 |
| A7 | ENSESTT00000037853 | | 7 | 74757541 | 74769401 |
| A7 | OTTHUMT00007006447 | HIP1 | 7 | 74775947 | 74840649 |
| A7 | ENSESTT00000037852 | | 7 | 74779537 | 74786565 |
| A7 | ENST00000320938 | HIP1 | 7 | 74779582 | 74980306 |
| A7 | ENSESTT00000037851 | | 7 | 74788329 | 74797481 |
| A7 | ENSESTT00000037850 | | 7 | 74799047 | 74804723 |
| A7 | ENSESTT00000037849 | | 7 | 74895820 | 74980379 |
| A7 | OTTHUMT00007007406 | Hs_7_c1160 | 7 | 74980208 | 74980327 |
| A7 | ENST00000005180 | CCL26 | 7 | 75010931 | 75013663 |
| A7 | ENSESTT00000040258 | | 7 | 75011028 | 75031153 |
| A7 | OTTHUMT00007006539 | SCYA26 | 7 | 75011100 | 75013583 |
| A7 | ENST00000222902 | CCL24 | 7 | 75053203 | 75055122 |
| A7 | OTTHUMT00007006535 | SCYA24 | 7 | 75053203 | 75055122 |
| A7 | OTTHUMT00007007412 | Hs_7_c1163 | 7 | 75077073 | 75077548 |
| A7 | ENST00000006777 | NM_020684 | 7 | 75120355 | 75130328 |
| A7 | OTTHUMT00007007126 | NPD007 | 7 | 75120355 | 75134177 |
| A7 | ENSESTT00000040185 | | 7 | 75120424 | 75130279 |
| A7 | ENSESTT00000040186 | | 7 | 75120432 | 75130279 |
| A7 | ENSESTT00000040257 | | 7 | 75120464 | 75130107 |
| A7 | ENST00000318622 | Q9UDT1 | 7 | 75120490 | 75122833 |
| A7 | ENSESTT00000040187 | | 7 | 75120579 | 75129992 |
| A7 | ENSESTT00000040195 | | 7 | 75156536 | 75222052 |
| A7 | ENSESTT00000040194 | | 7 | 75156536 | 75225046 |
| A7 | ENSESTT00000040192 | | 7 | 75156536 | 75225268 |
| A7 | ENSESTT00000040193 | | 7 | 75156536 | 75225268 |
| A7 | ENSESTT00000040188 | | 7 | 75156536 | 75226369 |
| A7 | ENSESTT00000040189 | | 7 | 75156536 | 75226369 |
| A7 | ENSESTT00000040190 | | 7 | 75156536 | 75226369 |
| A7 | ENSESTT00000040191 | | 7 | 75156536 | 75226369 |
| A7 | ENSESTT00000040202 | | 7 | 75156536 | 75226773 |
| A7 | ENSESTT00000040203 | | 7 | 75156536 | 75226773 |
| A7 | OTTHUMT00007007414 | Hs_7_c1165 | 7 | 75164520 | 75164746 |
| A7 | OTTHUMT00007006487 | mbhmh_h_73557902_74457901_m_133948896_13 | 7 | 75176809 | 75227888 |
| A7 | ENSESTT00000040200 | | 7 | 75195386 | 75225046 |
| A7 | ENSESTT00000040199 | | 7 | 75195386 | 75225268 |
| A7 | ENSESTT00000040197 | | 7 | 75195386 | 75226369 |
| A7 | ENSESTT00000040198 | | 7 | 75195386 | 75226369 |
| A7 | ENSESTT00000040196 | | 7 | 75195386 | 75226773 |
| A7 | ENST00000265302 | POR | 7 | 75195400 | 75227888 |
| A7 | ENSESTT00000040201 | | 7 | 75223628 | 75226773 |
| A7 | OTTHUMT00007007077 | TMPIT | 7 | 75228396 | 75236042 |
| A7 | ENSESTT00000040252 | | 7 | 75229120 | 75236046 |
| A7 | ENSESTT00000040253 | | 7 | 75229129 | 75236046 |
| A7 | ENSESTT00000040254 | | 7 | 75229182 | 75236046 |
| A7 | ENSESTT00000040255 | | 7 | 75229333 | 75236046 |
| A7 | ENSESTT00000040256 | | 7 | 75229371 | 75236046 |
| A7 | ENSESTT00000040248 | | 7 | 75237744 | 75255296 |
| A7 | ENSESTT00000040244 | | 7 | 75237744 | 75289349 |
| A7 | ENSESTT00000040245 | | 7 | 75237744 | 75289349 |
| A7 | ENSESTT00000040238 | | 7 | 75237744 | 75289395 |
| A7 | ENSESTT00000040239 | | 7 | 75237744 | 75289395 |
| A7 | ENSESTT00000040240 | | 7 | 75237744 | 75289395 |
| A7 | ENSESTT00000040236 | | 7 | 75237744 | 75295682 |
| A7 | ENSESTT00000040251 | | 7 | 75237749 | 75242410 |
| A7 | ENSESTT00000040250 | | 7 | 75237749 | 75246817 |
| A7 | ENSESTT00000040249 | | 7 | 75237749 | 75255296 |
| A7 | ENSESTT00000040247 | | 7 | 75237749 | 75263402 |
| A7 | ENSESTT00000040246 | | 7 | 75237749 | 75289349 |
| A7 | ENSESTT00000040241 | | 7 | 75237749 | 75289395 |
| A7 | ENSESTT00000040242 | | 7 | 75237749 | 75289395 |
| A7 | OTTHUMT00007006727 | MK-STYX | 7 | 75237749 | 75289410 |
| A7 | ENSESTT00000040237 | | 7 | 75237749 | 75295682 |
| A7 | ENST00000248600 | MSTY_HUMAN | 7 | 75237875 | 75271930 |
| A7 | ENSESTT00000040243 | | 7 | 75255146 | 75289370 |
| A7 | ENST00000315790 | | 7 | 75288063 | 75288355 |
| A7 | ENSESTT00000040204 | | 7 | 75289457 | 75308009 |
| A7 | ENST00000315758 | MDH2 | 7 | 75289482 | 75308017 |
| A7 | OTTHUMT00007007151 | MDH2 | 7 | 75289482 | 75308020 |
| A7 | ENSESTT00000040205 | | 7 | 75289536 | 75308009 |
| A7 | ENSESTT00000040206 | | 7 | 75305745 | 75308009 |
| A7 | OTTHUMT00007007750 | Hs_7_c3073 | 7 | 75312957 | 75313204 |
| A7 | OTTHUMT00007007439 | Hs_7_c1171 | 7 | 75315857 | 75316216 |
| A7 | OTTHUMT00007008045 | Hs_7_c5123 | 7 | 75340845 | 75342813 |
| A7 | OTTHUMT00007007443 | Hs_7_c1174 | 7 | 75349841 | 75373549 |
| A7 | ENST00000332057 | | 7 | 75354317 | 75354842 |
| A7 | OTTHUMT00007007445 | Hs_7_c1175 | 7 | 75354317 | 75354842 |
| A7 | OTTHUMT00007007446 | Hs_7_c1176 | 7 | 75390863 | 75390964 |
| A7 | OTTHUMT00007007447 | Hs_7_c1177 | 7 | 75415144 | 75418535 |
| A7 | ENSESTT00000040208 | | 7 | 75443300 | 75501454 |
| A7 | ENSESTT00000040207 | | 7 | 75443300 | 75506790 |
| A7 | ENSESTT00000040209 | | 7 | 75443307 | 75506790 |
| A7 | ENSESTT00000040210 | | 7 | 75476389 | 75501454 |
| A7 | ENST00000326382 | | 7 | 75476473 | 75522894 |
| A7 | OTTHUMT00007006504 | mbhmh_h_73557902_74457901_m_133948896_13 | 7 | 75476474 | 75527249 |
| A7 | ENSESTT00000040211 | | 7 | 75514460 | 75522893 |
| A7 | ENSESTT00000040212 | | 7 | 75523140 | 75527181 |
| A7 | ENST00000326284 | NM_153043 | 7 | 75524053 | 75528692 |
| A7 | ENSESTT00000040213 | | 7 | 75543994 | 75545679 |
| A7 | OTTHUMT00007006552 | HSPB1 | 7 | 75544012 | 75545701 |
| A7 | ENST00000248553 | HSPB1 | 7 | 75544012 | 75545702 |
| A7 | OTTHUMT00007006186 | YWHAG | 7 | 75568205 | 75600405 |
| A7 | ENST00000307630 | YWHAG | 7 | 75570983 | 75600214 |
| A7 | ENSESTT00000040235 | | 7 | 75570997 | 75600397 |
| A7 | ENST00000325070 | | 7 | 75602536 | 75603507 |
| A7 | ENST00000275560 | SRCRB4D | 7 | 75630735 | 75651095 |
| A7 | OTTHUMT00007006188 | SRCRB4D | 7 | 75630735 | 75651101 |
| A7 | ENSESTT00000040234 | | 7 | 75631056 | 75638966 |
| A7 | ENST00000297799 | Q96BF5 | 7 | 75631465 | 75635032 |
| A7 | OTTHUMT00007006555 | ZP3A | 7 | 75666341 | 75683471 |
| A7 | ENST00000257652 | ZP3 | 7 | 75666371 | 75683468 |
| A7 | ENSESTT00000040214 | | 7 | 75670977 | 75683476 |
| A7 | ENSESTT00000040215 | | 7 | 75674328 | 75683368 |
| A7 | ENSESTT00000040216 | | 7 | 75703078 | 75724019 |
| A7 | ENSESTT00000040217 | | 7 | 75703114 | 75724170 |
| A7 | ENSESTT00000040218 | | 7 | 75703116 | 75722103 |
| A7 | ENSESTT00000040219 | | 7 | 75703123 | 75724122 |
| A7 | ENSESTT00000040220 | | 7 | 75703136 | 75724190 |
| A7 | OTTHUMT00007006196 | DTX2 | 7 | 75703141 | 75747397 |
| A7 | ENST00000324432 | DTX2 | 7 | 75703143 | 75747007 |
| A7 | ENSESTT00000040221 | | 7 | 75703158 | 75721985 |
| A7 | OTTHUMT00007007472 | Hs_7_c1183 | 7 | 75711502 | 75712557 |
| A7 | ENST00000329896 | | 7 | 75711568 | 75712530 |
| A7 | ENSESTT00000040222 | | 7 | 75743571 | 75747401 |
| A7 | ENSESTT00000040223 | | 7 | 75751969 | 75756864 |
| A7 | ENSESTT00000040224 | | 7 | 75751972 | 75755467 |
| A7 | ENSESTT00000040225 | | 7 | 75752017 | 75756483 |
| A7 | ENST00000334348 | UPK3B | 7 | 75752059 | 75756610 |
| A7 | ENST00000257632 | UPK3B | 7 | 75752059 | 75756657 |
| A7 | ENSESTT00000040226 | | 7 | 75752161 | 75755394 |
| A7 | ENSESTT00000040227 | | 7 | 75752941 | 75756621 |
| A7 | ENST00000333674 | | 7 | 75763339 | 75774103 |
| A7 | OTTHUMT00007007476 | Hs_7_c1187 | 7 | 75765921 | 75774100 |
| A7 | ENST00000332397 | | 7 | 75772915 | 75781708 |
| A7 | OTTHUMT00007007156 | mbhmh_H_DJ1158B01_F218045.fgenesh2.2 | 7 | 75774096 | 75782865 |
| A7 | ENSESTT00000040233 | | 7 | 75775379 | 75781751 |
| A7 | ENST00000328339 | | 7 | 75777575 | 75780226 |
| A7 | OTTHUMT00007007474 | Hs_7_c1186 | 7 | 75791024 | 75792928 |
| A7 | ENSESTT00000040229 | | 7 | 75851392 | 75867091 |
| A7 | ENSESTT00000040228 | | 7 | 75851392 | 75867499 |
| A7 | ENSESTT00000040232 | | 7 | 75851392 | 75867499 |
| A7 | OTTHUMT00007006838 | POMZP3 | 7 | 75851394 | 75868655 |
| A7 | ENST00000310842 | POMZP3 | 7 | 75851570 | 75868652 |
| A7 | ENSESTT00000040230 | | 7 | 75852858 | 75867011 |
| A7 | ENSESTT00000040231 | | 7 | 75859587 | 75868162 |
| A7 | OTTHUMT00007007515 | Hs_7_c1194 | 7 | 75891805 | 75892303 |
| A7 | OTTHUMT00007007518 | Hs_7_c1196 | 7 | 76201256 | 76245716 |
| A7 | OTTHUMT00007007520 | Hs_7_c1197 | 7 | 76209601 | 76210656 |
| A7 | ENST00000331556 | | 7 | 76209667 | 76210629 |
| A7 | ENSESTT00000037296 | | 7 | 76214085 | 76218367 |
| A7 | ENST00000307569 | | 7 | 76220023 | 76231777 |
| A7 | ENST00000162863 | PMS2L11 | 7 | 76243584 | 76257202 |
| A7 | ENSESTT00000037258 | | 7 | 76243673 | 76245780 |
| A7 | ENSESTT00000037261 | | 7 | 76255577 | 76265170 |
| A7 | ENSESTT00000037259 | | 7 | 76257204 | 76440006 |
| A7 | OTTHUMT00007007328 | Hs_7_c1202 | 7 | 76260312 | 76295598 |
| A7 | ENST00000285792 | Q86WY7 | 7 | 76263908 | 76269847 |
| A7 | ENSESTT00000037292 | | 7 | 76264978 | 76266386 |
| A7 | OTTHUMT00007006602 | mfhmh_gw11359887.75188365.75365376.7.9e− | 7 | 76266095 | 76300760 |
| A7 | ENSESTT00000037291 | | 7 | 76266148 | 76294084 |
| A7 | ENSESTT00000037295 | | 7 | 76269559 | 76270744 |
| A7 | ENSESTT00000037294 | | 7 | 76271927 | 76273767 |
| A7 | ENSESTT00000037293 | | 7 | 76279424 | 76281262 |
| A7 | ENSESTT00000037290 | | 7 | 76294463 | 76300803 |
| A7 | ENST00000330572 | | 7 | 76297956 | 76299277 |
| A7 | OTTHUMT00007007330 | Hs_7_c1203 | 7 | 76313155 | 76313732 |
| A7 | ENSESTT00000037289 | | 7 | 76325407 | 76363754 |
| A7 | OTTHUMT00007006666 | mbhmh_h_75248517_76148516_m_19731738_198 | 7 | 76330887 | 76352916 |
| A7 | ENSESTT00000037288 | | 7 | 76351350 | 76363761 |
| A7 | ENST00000327285 | | 7 | 76356805 | 76357619 |
| A7 | OTTHUMT00007007332 | Hs_7_c1204 | 7 | 76356814 | 76357592 |
| A7 | ENSESTT00000037260 | | 7 | 76364104 | 76409308 |
| A7 | OTTHUMT00007006412 | mbax_nh_gi17389564 | 7 | 76437484 | 76441197 |
| A7 | ENST00000248598 | FGL2 | 7 | 76437495 | 76441207 |
| A7 | ENSESTT00000037287 | | 7 | 76437634 | 76441213 |
| A7 | OTTHUMT00007006667 | mbhmh_h_75248517_76148516_m_19731738_198 | 7 | 76466089 | 76536234 |
| A7 | ENST00000285871 | Q96MS1 | 7 | 76478324 | 76520191 |
| A7 | ENST00000257657 | Q9P223 | 7 | 76501336 | 76536578 |
| A7 | ENSESTT00000037262 | | 7 | 76501508 | 76523986 |
| A7 | ENSESTT00000037264 | | 7 | 76523852 | 76536589 |
| A7 | ENSESTT00000037263 | | 7 | 76523852 | 76571386 |
| A7 | ENSESTT00000037284 | | 7 | 76552119 | 76571745 |
| A7 | ENST00000257626 | Q9Y4L9 | 7 | 76552120 | 76568852 |
| A7 | OTTHUMT00007007104 | mbhmh_nh_h_75248517_76148516_m_18931739_{—} | 7 | 76552726 | 76605693 |
| A7 | ENSESTT00000037285 | | 7 | 76552993 | 76571745 |
| A7 | ENSESTT00000037286 | | 7 | 76562091 | 76567945 |
| A7 | ENST00000334003 | Q8ND73 | 7 | 76571601 | 76647477 |
| A7 | ENSESTT00000037283 | | 7 | 76571643 | 76596756 |
| A7 | OTTHUMT00007007105 | mbhmh_nh_h_75248517_76148516_m_18931739_{—} | 7 | 76611431 | 76653274 |
| A7 | ENSESTT00000037282 | | 7 | 76638369 | 76657235 |
| A7 | ENST00000310324 | Q9BXE6 | 7 | 76751393 | 76751767 |
| A7 | ENSESTT00000037265 | | 7 | 76778862 | 76839272 |
| A7 | OTTHUMT00007006825 | PTPN12 | 7 | 76778886 | 76881437 |
| A7 | ENSESTT00000037266 | | 7 | 76778907 | 76842330 |
| A7 | ENST00000248594 | PTPN12 | 7 | 76778915 | 76880648 |
| A7 | ENSESTT00000037267 | | 7 | 76805409 | 76842330 |
| A7 | ENSESTT00000037269 | | 7 | 76848601 | 76881437 |
| A7 | ENSESTT00000037270 | | 7 | 76848601 | 76881437 |
| A7 | ENSESTT00000037271 | | 7 | 76848601 | 76881437 |
| A7 | ENSESTT00000037268 | | 7 | 76848601 | 76895005 |
| A7 | ENSESTT00000037272 | | 7 | 76873632 | 76881437 |
| A7 | ENSESTT00000037281 | | 7 | 76875004 | 76889253 |
| A7 | ENSESTT00000037273 | | 7 | 76878133 | 76881437 |
| A7 | ENSESTT00000037274 | | 7 | 76878340 | 76880309 |
| A7 | ENSESTT00000037278 | | 7 | 76900557 | 76937634 |
| A7 | ENSESTT00000037275 | | 7 | 76910887 | 76912750 |
| A7 | ENSESTT00000037279 | | 7 | 76925217 | 76937634 |
| A7 | ENSESTT00000037280 | | 7 | 76925311 | 76937620 |
| A7 | ENST00000334955 | Q86X48 | 7 | 76937838 | 76977836 |
| A7 | OTTHUMT00007006307 | mbhmh_h_75248517_76148516_m_18931739_198 | 7 | 76937838 | 77020536 |
| A7 | ENSESTT00000037277 | | 7 | 76977776 | 76991120 |
| A7 | ENSESTT00000037276 | | 7 | 76990885 | 77010048 |
| A7 | ENSESTT00000031431 | | 7 | 77010043 | 77021059 |
| A7 | OTTHUMT00007006265 | DC32 | 7 | 77035083 | 77039701 |
| A7 | ENST00000257663 | C7orf35 | 7 | 77035084 | 77039798 |
| A7 | ENSESTT00000031444 | | 7 | 77035096 | 77039946 |
| A7 | ENSESTT00000031445 | | 7 | 77035258 | 77039797 |
| A7 | OTTHUMT00007006310 | mbhmh_h_75248517_76148516_m_18931739_198 | 7 | 77081624 | 77196246 |
| A7 | ENST00000248550 | Q8TBW4 | 7 | 77096101 | 77195265 |
| A8 | ENST00000256653 | MAN1A2 | 1 | 117256452 | 117415542 |
| A8 | ENSESTT00000003501 | | 1 | 117256479 | 117412324 |
| A8 | ENSESTT00000003500 | | 1 | 117256479 | 117414355 |
| A8 | ENSESTT00000003502 | | 1 | 117331232 | 117385841 |
| A8 | ENSESTT00000003523 | | 1 | 117485833 | 117494713 |
| A8 | ENSESTT00000003503 | | 1 | 117494976 | 117512577 |
| A8 | ENST00000328500 | NM_017709 | 1 | 117495052 | 117517367 |
| A8 | ENST00000313132 | | 1 | 117529727 | 117530632 |
| A8 | ENST00000334351 | PNRC2 | 1 | 117667090 | 117667509 |
| A8 | ENSESTT00000003522 | | 1 | 117667118 | 117667900 |
| A8 | ENSESTT00000003520 | | 1 | 117759186 | 117818631 |
| A8 | ENSESTT00000003521 | | 1 | 117759281 | 117818631 |
| A8 | ENST00000263166 | GDAP2 | 1 | 117759572 | 117818591 |
| A8 | ENSESTT00000003504 | | 1 | 117818779 | 117823687 |
| A8 | ENST00000309112 | Q9H141 | 1 | 117822324 | 117848451 |
| A8 | ENST00000183319 | WDR3 | 1 | 117822324 | 117849059 |
| A8 | ENSESTT00000003505 | | 1 | 117831482 | 117849104 |
| A8 | ENSESTT00000003518 | | 1 | 117842855 | 117853814 |
| A8 | ENSESTT00000003515 | | 1 | 117842859 | 117859122 |
| A8 | ENSESTT00000003519 | | 1 | 117842865 | 117852907 |
| A8 | ENSESTT00000003517 | | 1 | 117842865 | 117855703 |
| A8 | ENST00000286203 | Q8NAZ1 | 1 | 117842866 | 118074161 |
| A8 | ENSESTT00000003516 | | 1 | 117842868 | 117855764 |
| A8 | ENSESTT00000003513 | | 1 | 117861020 | 117880531 |
| A8 | ENSESTT00000003514 | | 1 | 117862486 | 117876996 |
| A8 | ENSESTT00000003512 | | 1 | 117885713 | 117905184 |
| A8 | ENSESTT00000003510 | | 1 | 117917336 | 117943048 |
| A8 | ENSESTT00000003511 | | 1 | 117920750 | 117928350 |
| A8 | ENSESTT00000003509 | | 1 | 117943055 | 117962920 |
| A8 | ENSESTT00000003507 | | 1 | 117943058 | 117970490 |
| A8 | ENSESTT00000003508 | | 1 | 117962945 | 117970300 |
| A8 | ENSESTT00000003506 | | 1 | 117974982 | 117976166 |
| A8 | ENSESTT00000003436 | | 1 | 118039391 | 118074185 |
| A8 | ENST00000334368 | Q9UN81 | 1 | 118742261 | 118743277 |
| A8 | ENST00000207157 | TBX15 | 1 | 118772051 | 118820834 |
| A8 | ENSESTT00000003435 | | 1 | 118889344 | 118890396 |
| A8 | ENSESTT00000003434 | | 1 | 118889622 | 118890409 |
| A8 | ENST00000235521 | WARS2 | 1 | 118920227 | 119029659 |
| A8 | ENSESTT00000003433 | | 1 | 118921825 | 118934670 |
| A8 | ENSESTT00000003432 | | 1 | 118922024 | 119029623 |
| A8 | ENST00000333224 | | 1 | 119015816 | 119016295 |
| A8 | ENSESTT00000003526 | | 1 | 119035413 | 119036037 |
| A8 | ENST00000330630 | | 1 | 119108318 | 119108923 |
| A8 | ENSESTT00000003527 | | 1 | 119217196 | 119219818 |
| A8 | ENSESTT00000003528 | | 1 | 119217256 | 119245186 |
| A8 | ENSESTT00000003529 | | 1 | 119257783 | 119275698 |
| A8 | ENSESTT00000003531 | | 1 | 119257783 | 119281249 |
| A8 | ENSESTT00000003530 | | 1 | 119269568 | 119281249 |
| A8 | ENST00000325945 | HAO2 | 1 | 119270090 | 119282844 |
| A8 | ENST00000331050 | Q8TDP9 | 1 | 119304149 | 119403649 |
| A8 | ENSESTT00000003533 | | 1 | 119304154 | 119311277 |
| A8 | ENSESTT00000003532 | | 1 | 119304154 | 119311779 |
| A8 | ENSESTT00000003534 | | 1 | 119304167 | 119308609 |
| A8 | ENST00000303184 | HSD3B2 | 1 | 119304424 | 119311624 |
| A8 | ENST00000333709 | Q8TDP9 | 1 | 119304427 | 119403649 |
| A8 | ENST00000332017 | Q8TDP9 | 1 | 119304427 | 119404056 |
| A8 | ENST00000256586 | Q9H1N0 | 1 | 119328005 | 119334823 |
| A8 | ENST00000286193 | Q9H1M9 | 1 | 119356225 | 119362218 |
| A8 | ENST00000331024 | | 1 | 119385611 | 119385839 |
| A8 | ENSESTT00000003535 | | 1 | 119396243 | 119404058 |
| A8 | ENSESTT00000003536 | | 1 | 119396244 | 119397374 |
| A8 | ENSESTT00000003537 | | 1 | 119396322 | 119404058 |
| A8 | ENST00000235547 | HSD3B1 | 1 | 119396481 | 119403649 |
| A8 | ENST00000335580 | Q9UDK8 | 1 | 119456897 | 119461071 |
| A8 | ENST00000331009 | | 1 | 119485270 | 119485838 |
| A8 | ENST00000335229 | Q9UD07 | 1 | 119494238 | 119494381 |
| A8 | ENST00000271263 | Q96IT2 | 1 | 119511709 | 119512401 |
| A8 | ENSESTT00000003538 | | 1 | 119600896 | 119633222 |
| A8 | ENSESTT00000003539 | | 1 | 119600896 | 119633222 |
| A8 | ENST00000263167 | PHGDH | 1 | 119601027 | 119633206 |
| A8 | ENSESTT00000003540 | | 1 | 119615847 | 119633222 |
| A8 | ENSESTT00000003541 | | 1 | 119615847 | 119633222 |
| A8 | ENSESTT00000003542 | | 1 | 119616052 | 119619075 |
| A8 | ENSESTT00000003543 | | 1 | 119626114 | 119633222 |
| A8 | ENST00000256633 | HMGCS2 | 1 | 119637386 | 119657898 |
| A8 | ENSESTT00000003552 | | 1 | 119637543 | 119657890 |
| A8 | ENSESTT00000003553 | | 1 | 119648894 | 119657887 |
| A8 | ENSESTT00000003544 | | 1 | 119653323 | 119657877 |
| A8 | ENST00000324032 | Q8NER6 | 1 | 119683168 | 119700499 |
| A8 | ENSESTT00000003551 | | 1 | 119683421 | 119700465 |
| A8 | ENSESTT00000003550 | | 1 | 119683593 | 119692170 |
| A8 | ENST00000256585 | REG4 | 1 | 119683622 | 119697773 |
| A8 | ENSESTT00000003549 | | 1 | 119687553 | 119697870 |
| A9 | OTTHUMT00006006260 | SLC26A8-001 | 6 | 35958152 | 36039212 |
| A9 | ENST00000229784 | SLC26A8 | 6 | 35958327 | 36034339 |
| A9 | ENST00000310888 | SLC26A8 | 6 | 35958532 | 36034339 |
| A9 | ENSESTT00000033005 | | 6 | 36012429 | 36039125 |
| A9 | ENSESTT00000032935 | | 6 | 36042425 | 36067456 |
| A9 | OTTHUMT00006006277 | MAPK14-002 | 6 | 36042428 | 36123079 |
| A9 | OTTHUMT00006006276 | MAPK14-001 | 6 | 36042428 | 36125390 |
| A9 | ENST00000229794 | NM_139014 | 6 | 36042790 | 36122248 |
| A9 | ENST00000229795 | MAPK14 | 6 | 36042790 | 36123079 |
| A9 | ENST00000310795 | MAPK14 | 6 | 36042790 | 36123079 |
| A9 | ENSESTT00000032936 | | 6 | 36090530 | 36124509 |
| A9 | OTTHUMT00006006258 | dJ179N16.3-001 | 6 | 36106623 | 36107278 |
| A9 | OTTHUMT00006006270 | MAPK13-005 | 6 | 36142441 | 36150464 |
| A9 | ENSESTT00000032938 | | 6 | 36142441 | 36154698 |
| A9 | ENSESTT00000032937 | | 6 | 36142441 | 36155902 |
| A9 | OTTHUMT00006006268 | MAPK13-003 | 6 | 36144953 | 36154312 |
| A9 | OTTHUMT00006006266 | MAPK13-001 | 6 | 36144953 | 36154697 |
| A9 | ENST00000211287 | MAPK13 | 6 | 36145117 | 36154697 |
| A9 | ENSESTT00000032940 | | 6 | 36145142 | 36154698 |
| A9 | ENSESTT00000032939 | | 6 | 36145142 | 36155902 |
| A9 | ENSESTT00000032941 | | 6 | 36145145 | 36151536 |
| A9 | ENSESTT00000032943 | | 6 | 36145202 | 36154698 |
| A9 | ENSESTT00000032942 | | 6 | 36145202 | 36155902 |
| A9 | ENSESTT00000032944 | | 6 | 36145273 | 36146034 |
| A9 | OTTHUMT00006006269 | MAPK13-004 | 6 | 36145295 | 36153839 |
| A9 | OTTHUMT00006006267 | MAPK13-002 | 6 | 36145501 | 36151147 |
| A9 | OTTHUMT00006006280 | BRPF3-001 | 6 | 36211405 | 36247418 |
| A9 | ENST00000322766 | BRPF3 | 6 | 36211405 | 36247421 |
| A9 | ENSESTT00000032945 | | 6 | 36212028 | 36215240 |
| A9 | ENSESTT00000032946 | | 6 | 36215902 | 36224498 |
| A9 | OTTHUMT00006006281 | BRPF3-002 | 6 | 36225130 | 36247418 |
| A9 | ENST00000211291 | Q9NWM1 | 6 | 36225982 | 36245352 |
| A9 | ENSESTT00000032947 | | 6 | 36228665 | 36232584 |
| A9 | ENSESTT00000032948 | | 6 | 36239919 | 36246148 |
| A9 | OTTHUMT00006006284 | dJ50J22.1-001 | 6 | 36285116 | 36310051 |
| A9 | ENST00000312917 | PNPLA1 | 6 | 36306032 | 36323227 |
| A9 | OTTHUMT00006006294 | dJ50J22.5-001 | 6 | 36317034 | 36327443 |
| A9 | OTTHUMT00006006290 | dJ50J22.3-001 | 6 | 36331805 | 36334066 |
| A9 | OTTHUMT00006006286 | dJ50J22.2-001 | 6 | 36380826 | 36402415 |
| A9 | OTTHUMT00006006287 | dJ50J22.2-002 | 6 | 36380826 | 36402415 |
| A9 | ENST00000229480 | ETV7 | 6 | 36380827 | 36402349 |
| A9 | ENSESTT00000033001 | | 6 | 36390551 | 36402400 |
| A9 | OTTHUMT00006006292 | dJ50J22.4-001 | 6 | 36401463 | 36406626 |
| A9 | ENST00000316266 | NM_152990 | 6 | 36405183 | 36415166 |
| A9 | OTTHUMT00006006296 | dJ347L7.1-001 | 6 | 36415095 | 36457521 |
| A9 | ENSESTT00000032949 | | 6 | 36457399 | 36458352 |
| A9 | ENST00000265344 | C6orf69 | 6 | 36457399 | 36505168 |
| A9 | OTTHUMT00006006298 | dJ108K11.3-001 | 6 | 36457619 | 36505775 |
| A9 | ENSESTT00000032950 | | 6 | 36484759 | 36499460 |
| A9 | ENSESTT00000032952 | | 6 | 36499410 | 36503191 |
| A9 | ENSESTT00000032951 | | 6 | 36499410 | 36505772 |
| A9 | ENSESTT00000032953 | | 6 | 36501514 | 36505772 |
| A9 | OTTHUMT00006006300 | STK38-001 | 6 | 36508524 | 36562102 |
| A9 | ENST00000229812 | STK38 | 6 | 36508531 | 36562102 |
| A9 | ENSESTT00000032999 | | 6 | 36510468 | 36522221 |
| A9 | ENSESTT00000032996 | | 6 | 36530077 | 36562102 |
| A9 | ENSESTT00000032955 | | 6 | 36608994 | 36616435 |
| A9 | ENSESTT00000032954 | | 6 | 36608994 | 36617545 |
| A9 | OTTHUMT00006006302 | SFRS3-001 | 6 | 36609000 | 36618064 |
| A9 | OTTHUMT00006006303 | SFRS3-002 | 6 | 36609023 | 36616672 |
| A9 | ENST00000244437 | SFRS3 | 6 | 36611395 | 36617408 |
| A9 | ENST00000317631 | | 6 | 36688401 | 36689903 |
| A9 | OTTHUMT00006006306 | dJ193M11.1-001 | 6 | 36688449 | 36689902 |
| A9 | OTTHUMT00006012688 | CDKN1A-005 | 6 | 36691160 | 36699143 |
| A9 | ENSESTT00000032956 | | 6 | 36692420 | 36701950 |
| A9 | OTTHUMT00006012684 | CDKN1A-001 | 6 | 36693290 | 36701971 |
| A9 | ENST00000244741 | CDKN1A | 6 | 36693342 | 36701963 |
| A9 | OTTHUMT00006012685 | CDKN1A-002 | 6 | 36693353 | 36700556 |
| A9 | ENSESTT00000032957 | | 6 | 36693354 | 36701950 |
| A9 | OTTHUMT00006012687 | CDKN1A-004 | 6 | 36693358 | 36699130 |
| A9 | OTTHUMT00006012686 | CDKN1A-003 | 6 | 36693403 | 36695312 |
| A9 | ENSESTT00000032958 | | 6 | 36693403 | 36695312 |
| A9 | OTTHUMT00006011804 | dJ431A14.3-001 | 6 | 36737227 | 36743433 |
| A9 | ENST00000229824 | | 6 | 36737257 | 36745385 |
| A9 | ENST00000310390 | Q8TDV1 | 6 | 36751637 | 36752338 |
| A9 | OTTHUMT00006006308 | dJ431A14.4-001 | 6 | 36751682 | 36752618 |
| A9 | OTTHUMT00006006311 | dJ431A14.5-002 | 6 | 36755407 | 36772013 |
| A9 | ENST00000244751 | CPNE5 | 6 | 36755410 | 36854008 |
| A9 | OTTHUMT00006006310 | dJ431A14.5-001 | 6 | 36755410 | 36854633 |
| A9 | ENSESTT00000032989 | | 6 | 36758313 | 36772015 |
| A9 | OTTHUMT00006006312 | dJ431A14.5-003 | 6 | 36760019 | 36772013 |
| A9 | ENSESTT00000032990 | | 6 | 36760019 | 36772013 |
| A9 | ENSESTT00000032987 | | 6 | 36760019 | 36780337 |
| A9 | ENSESTT00000032988 | | 6 | 36760024 | 36772025 |
| A9 | OTTHUMT00006006351 | PPIL1-002 | 6 | 36869458 | 36873266 |
| A9 | OTTHUMT00006006350 | PPIL1-001 | 6 | 36869458 | 36889655 |
| A9 | ENST00000244367 | PPIL1 | 6 | 36869463 | 36889629 |
| A9 | ENSESTT00000032983 | | 6 | 36870099 | 36889655 |
| A9 | ENSESTT00000032984 | | 6 | 36870254 | 36886506 |
| A9 | OTTHUMT00006006316 | dJ90K10.2-001 | 6 | 36886501 | 36939186 |
| A9 | OTTHUMT00006006319 | dJ90K10.2-004 | 6 | 36900485 | 36931139 |
| A9 | OTTHUMT00006006317 | dJ90K10.2-002 | 6 | 36900495 | 36938153 |
| A9 | OTTHUMT00006006318 | dJ90K10.2-003 | 6 | 36900495 | 36938153 |
| A9 | ENST00000314503 | C6orf89 | 6 | 36900495 | 36941175 |
| A9 | ENSESTT00000032959 | | 6 | 36900540 | 36938610 |
| A9 | OTTHUMT00006006320 | dJ90K10.2-005 | 6 | 36900544 | 36938203 |
| A9 | ENSESTT00000032960 | | 6 | 36900558 | 36929229 |
| A9 | ENSESTT00000032961 | | 6 | 36909132 | 36938610 |
| A9 | OTTHUMT00006006326 | dJ90K10.3-001 | 6 | 36943210 | 36943499 |
| A9 | OTTHUMT00006006328 | dJ90K10.4-001 | 6 | 36954718 | 36959306 |
| A9 | ENST00000297048 | PI16 | 6 | 36969064 | 36979463 |
| A9 | OTTHUMT00006006330 | dJ90K10.5-001 | 6 | 36969064 | 36979468 |
| A9 | ENSESTT00000032962 | | 6 | 36969360 | 36974006 |
| A9 | ENSESTT00000032963 | | 6 | 36969368 | 36978833 |
| A9 | ENSESTT00000032964 | | 6 | 36969383 | 36978833 |
| A9 | OTTHUMT00006006331 | dJ90K10.5-002 | 6 | 36977621 | 36979298 |
| A9 | OTTHUMT00006006335 | dJ90K10.6-002 | 6 | 36982772 | 36991863 |
| A9 | OTTHUMT00006006341 | dJ90K10.6-008 | 6 | 36982772 | 37000929 |
| A9 | OTTHUMT00006006334 | dJ90K10.6-001 | 6 | 36982772 | 37000929 |
| A9 | ENSESTT00000032969 | | 6 | 36983213 | 36992788 |
| A9 | ENSESTT00000032965 | | 6 | 36983213 | 37000795 |
| A9 | ENSESTT00000032966 | | 6 | 36983310 | 37000795 |
| A9 | ENSESTT00000032967 | | 6 | 36983310 | 37000795 |
| A9 | OTTHUMT00006006340 | dJ90K10.6-007 | 6 | 36983368 | 37000612 |
| A9 | ENST00000259958 | MTCH1 | 6 | 36983368 | 37000747 |
| A9 | OTTHUMT00006006337 | dJ90K10.6-004 | 6 | 36983391 | 36985547 |
| A9 | OTTHUMT00006006336 | dJ90K10.6-003 | 6 | 36983543 | 36992304 |
| A9 | OTTHUMT00006006338 | dJ90K10.6-005 | 6 | 36984097 | 36987385 |
| A9 | ENSESTT00000032968 | | 6 | 36984669 | 37000795 |
| A9 | OTTHUMT00006006339 | dJ90K10.6-006 | 6 | 36984687 | 36987307 |
| A9 | OTTHUMT00006006356 | FGD2-001 | 6 | 37020277 | 37042929 |
| A9 | ENSESTT00000035539 | | 6 | 37020289 | 37026654 |
| A9 | ENSESTT00000035540 | | 6 | 37020301 | 37028317 |
| A9 | ENST00000274963 | FGD2 | 6 | 37020330 | 37043169 |
| A9 | ENSESTT00000035541 | | 6 | 37040439 | 37042882 |
| A9 | OTTHUMT00006006354 | dJ405J24.2-001 | 6 | 37059462 | 37060032 |
| A9 | ENST00000297147 | O95101 | 6 | 37059560 | 37059820 |
| A9 | OTTHUMT00006006358 | dJ441G21.1-001 | 6 | 37105853 | 37106493 |
| A9 | ENST00000310055 | | 6 | 37105943 | 37106437 |
| A9 | ENSESTT00000035542 | | 6 | 37184786 | 37185779 |
| A9 | ENSESTT00000035543 | | 6 | 37184786 | 37185779 |
| A9 | OTTHUMT00006012708 | PIM1-003 | 6 | 37184834 | 37190057 |
| A9 | ENST00000259722 | PIM1 | 6 | 37184841 | 37190057 |
| A9 | ENSESTT00000035544 | | 6 | 37185789 | 37190059 |
| A9 | OTTHUMT00006012709 | PIM1-004 | 6 | 37187099 | 37188878 |
| A9 | ENSESTT00000035545 | | 6 | 37187103 | 37190059 |
| A9 | OTTHUMT00006012706 | PIM1-001 | 6 | 37187125 | 37188943 |
| A9 | ENSESTT00000035546 | | 6 | 37187125 | 37190059 |
| A9 | OTTHUMT00006012707 | PIM1-002 | 6 | 37187627 | 37190057 |
| A9 | OTTHUMT00006006360 | dJ355M6.2-001 | 6 | 37226811 | 37272786 |
| A9 | ENST00000316899 | Q8TC54 | 6 | 37226812 | 37233661 |
| A9 | OTTHUMT00006006361 | dJ355M6.2-002 | 6 | 37227067 | 37272267 |
| A9 | ENST00000316909 | NM_145316 | 6 | 37229829 | 37233661 |
| A9 | ENSESTT00000035577 | | 6 | 37233261 | 37272786 |
| A9 | OTTHUMT00006006364 | dJ744I24.2-001 | 6 | 37272403 | 37347601 |
| A9 | ENSESTT00000035547 | | 6 | 37272498 | 37294209 |
| A9 | ENST00000229492 | C6orf197 | 6 | 37298967 | 37347600 |
| A9 | ENSESTT00000035548 | | 6 | 37331324 | 37347601 |
| A9 | ENSESTT00000035549 | | 6 | 37331816 | 37339289 |
| A9 | OTTHUMT00006006366 | RNF8-001 | 6 | 37368684 | 37409364 |
| A9 | ENSESTT00000035550 | | 6 | 37368716 | 37386205 |
| A9 | ENST00000229866 | RNF8 | 6 | 37368796 | 37395990 |
| A9 | ENSESTT00000035551 | | 6 | 37383509 | 37405872 |
| A9 | OTTHUMT00006006367 | RNF8-002 | 6 | 37383520 | 37391647 |
| A9 | ENSESTT00000035554 | | 6 | 37383557 | 37405862 |
| A9 | ENSESTT00000035552 | | 6 | 37383557 | 37405872 |
| A9 | ENSESTT00000035553 | | 6 | 37383557 | 37405872 |
| A9 | ENSESTT00000035555 | | 6 | 37391555 | 37405872 |
| A9 | ENSESTT00000035556 | | 6 | 37447848 | 37474233 |
| A9 | OTTHUMT00006006384 | dJ153P14.1-009 | 6 | 37447850 | 37458714 |
| A9 | ENSESTT00000035557 | | 6 | 37447850 | 37474233 |
| A9 | OTTHUMT00006006377 | dJ153P14.1-002 | 6 | 37447851 | 37476692 |
| A9 | OTTHUMT00006006376 | dJ153P14.1-001 | 6 | 37447851 | 37496137 |
| A9 | ENST00000259729 | NM_015050 | 6 | 37450261 | 37496137 |
| A9 | OTTHUMT00006006383 | dJ153P14.1-008 | 6 | 37467109 | 37473320 |
| A9 | ENSESTT00000035558 | | 6 | 37474283 | 37488203 |
| A9 | OTTHUMT00006006378 | dJ153P14.1-003 | 6 | 37474290 | 37476703 |
| A9 | ENSESTT00000035559 | | 6 | 37474290 | 37476703 |
| A9 | OTTHUMT00006006380 | dJ153P14.1-005 | 6 | 37477502 | 37495903 |
| A9 | OTTHUMT00006006379 | dJ153P14.1-004 | 6 | 37488199 | 37493826 |
| A9 | ENSESTT00000035560 | | 6 | 37489154 | 37496138 |
| A9 | ENSESTT00000035561 | | 6 | 37489159 | 37493872 |
| A9 | OTTHUMT00006006382 | dJ153P14.1-007 | 6 | 37489950 | 37497458 |
| A9 | ENSESTT00000035562 | | 6 | 37489950 | 37497460 |
| A9 | OTTHUMT00006006381 | dJ153P14.1-006 | 6 | 37489976 | 37495777 |
| A9 | OTTHUMT00006006394 | dJ153P14.2-001 | 6 | 37497551 | 37514553 |
| A9 | ENSESTT00000035575 | | 6 | 37497563 | 37514525 |
| A9 | ENST00000259975 | Q9P0B6 | 6 | 37497563 | 37514553 |
| A9 | ENSESTT00000035576 | | 6 | 37497569 | 37499495 |
| A9 | ENSESTT00000035574 | | 6 | 37497716 | 37514544 |
| A9 | OTTHUMT00006006395 | dJ153P14.2-002 | 6 | 37497716 | 37514553 |
| A9 | OTTHUMT00006006370 | dJ153P14.3-001 | 6 | 37558184 | 37561386 |
| A9 | ENSESTT00000035573 | | 6 | 37558184 | 37561393 |
| A9 | OTTHUMT00006006372 | dJ153P14.4-001 | 6 | 37561183 | 37562345 |
| A9 | ENSESTT00000035563 | | 6 | 37564634 | 37565497 |
| A9 | OTTHUMT00006006374 | dJ153P14.5-001 | 6 | 37564635 | 37565491 |
| A9 | ENSESTT00000035571 | | 6 | 37651734 | 37660878 |
| A9 | OTTHUMT00006006400 | dJ402N21.2-001 | 6 | 37651734 | 37669174 |
| A9 | ENST00000297153 | MDGA1 | 6 | 37651999 | 37711442 |
| A9 | ENST00000229875 | Q8NBE3 | 6 | 37652690 | 37660859 |
| A9 | ENSESTT00000035572 | | 6 | 37653259 | 37656528 |
| A9 | ENSESTT00000035568 | | 6 | 37663817 | 37669088 |
| A9 | ENSESTT00000035570 | | 6 | 37664502 | 37666973 |
| A9 | ENSESTT00000035569 | | 6 | 37664502 | 37669088 |
| A9 | OTTHUMT00006006398 | dJ402N21.1-001 | 6 | 37670396 | 37711576 |
| A9 | ENSESTT00000035567 | | 6 | 37672943 | 37711576 |
| A9 | OTTHUMT00006006402 | dJ441A12.1-001 | 6 | 37830408 | 37833849 |
| A9 | ENSESTT00000035566 | | 6 | 37833212 | 37833973 |
| A9 | ENSESTT00000035564 | | 6 | 37834162 | 37944634 |
| A9 | OTTHUMT00006006406 | TEX27-001 | 6 | 37834162 | 38169252 |
| A9 | ENSESTT00000035565 | | 6 | 37834188 | 38166902 |
| A9 | ENST00000287218 | TEX27 | 6 | 37834577 | 38167020 |
| A9 | OTTHUMT00006006407 | TEX27-002 | 6 | 37944570 | 38097088 |
| A9 | ENSESTT00000028267 | | 6 | 38016900 | 38018551 |
| A9 | OTTHUMT00006006404 | bA420A21.1-001 | 6 | 38017463 | 38017944 |
| A9 | OTTHUMT00006006408 | TEX27-003 | 6 | 38076242 | 38077291 |
| A9 | ENSESTT00000028247 | | 6 | 38076350 | 38168417 |
| A9 | OTTHUMT00006006409 | TEX27-004 | 6 | 38131203 | 38156962 |
| A9 | OTTHUMT00006006414 | dJ295F6.2-001 | 6 | 38177432 | 38178435 |
| A9 | OTTHUMT00006006425 | dJ322I12.1-004 | 6 | 38185948 | 38654445 |
| A9 | OTTHUMT00006006416 | dJ322I12.2-001 | 6 | 38188304 | 38191924 |
| A9 | OTTHUMT00006006426 | dJ322I12.1-005 | 6 | 38189588 | 38359724 |
| A9 | ENSESTT00000028266 | | 6 | 38189682 | 38271074 |
| A9 | ENST00000320902 | Q8NAH5 | 6 | 38193995 | 38194495 |
| A9 | OTTHUMT00006006423 | dJ322I12.1-002 | 6 | 38302912 | 38594901 |
| A9 | ENSESTT00000023248 | | 6 | 38496227 | 38497741 |
| A9 | OTTHUMT00006006418 | bA430C17.1-001 | 6 | 38496323 | 38497162 |
| A9 | OTTHUMT00006006422 | dJ322I12.1-001 | 6 | 38592231 | 38610698 |
| A9 | OTTHUMT00006006420 | dJ319M7.2-001 | 6 | 38601954 | 38603160 |
| A9 | ENST00000328403 | BTBD9 | 6 | 38607118 | 38612725 |
| A9 | OTTHUMT00006006424 | dJ322I12.1-003 | 6 | 38608899 | 38654542 |
| A9 | OTTHUMT00006006435 | GLO1-002 | 6 | 38690574 | 38697846 |
| A9 | OTTHUMT00006006434 | GLO1-001 | 6 | 38690574 | 38717772 |
| A9 | ENST00000244746 | GLO1 | 6 | 38690575 | 38717772 |
| A9 | ENSESTT00000028264 | | 6 | 38690577 | 38717784 |
| A9 | ENSESTT00000028262 | | 6 | 38691212 | 38699147 |
| A9 | ENSESTT00000028258 | | 6 | 38691212 | 38717784 |
| A9 | ENSESTT00000028265 | | 6 | 38691212 | 38717784 |
| A9 | ENSESTT00000028263 | | 6 | 38691365 | 38696733 |
| A9 | ENSESTT00000028259 | | 6 | 38696639 | 38717784 |
| A9 | ENSESTT00000028260 | | 6 | 38696641 | 38717784 |
| A9 | ENSESTT00000028261 | | 6 | 38697434 | 38717784 |
| A9 | OTTHUMT00006006432 | dJ503A6.2-001 | 6 | 38728682 | 38729848 |
| A9 | ENSESTT00000028257 | | 6 | 38728682 | 38729848 |
| A9 | ENST00000327475 | Q8IU65 | 6 | 38729972 | 38749145 |
| A9 | OTTHUMT00006012038 | DNAH8-003 | 6 | 38737477 | 38986711 |
| A9 | OTTHUMT00006012037 | DNAH8-002 | 6 | 38737477 | 39045150 |
| A9 | OTTHUMT00006012036 | DNAH8-001 | 6 | 38737753 | 39045156 |
| A9 | ENST00000244699 | DNAH8 | 6 | 38749146 | 39045422 |
| A9 | OTTHUMT00006011850 | dJ217P22.2-001 | 6 | 38777536 | 38779325 |
| A9 | ENSESTT00000028249 | | 6 | 38872332 | 38878623 |
| A9 | OTTHUMT00006012039 | DNAH8-004 | 6 | 38878600 | 38885121 |
| A9 | ENSESTT00000028250 | | 6 | 38887623 | 38888176 |
| A9 | OTTHUMT00006006438 | dJ207H1.3-001 | 6 | 38937660 | 38946002 |
| A9 | OTTHUMT00006006439 | dJ207H1.3-002 | 6 | 38937660 | 38967730 |
| A9 | ENSESTT00000028255 | | 6 | 38939657 | 38948048 |
| A9 | ENSESTT00000028251 | | 6 | 38940722 | 38944241 |
| A9 | OTTHUMT00006011974 | dJ207H1.2-001 | 6 | 38942662 | 38942784 |
| A9 | OTTHUMT00006006440 | dJ207H1.3-003 | 6 | 38943981 | 38947548 |
| A9 | ENSESTT00000028256 | | 6 | 38943981 | 38947548 |
| A9 | ENSESTT00000028254 | | 6 | 38958559 | 38967720 |
| A9 | ENSESTT00000028252 | | 6 | 38966101 | 38988421 |
| A9 | ENSESTT00000028253 | | 6 | 38998885 | 39027120 |
| A9 | ENSESTT00000028268 | | 6 | 39027083 | 39045160 |
| A9 | OTTHUMT00006006452 | dJ202I21.3-001 | 6 | 39054234 | 39054616 |
| A9 | OTTHUMT00006006444 | GLP1R-001 | 6 | 39063429 | 39102374 |
| A9 | ENST00000229900 | GLP1R | 6 | 39063472 | 39100704 |
| A9 | ENSESTT00000028298 | | 6 | 39118691 | 39124040 |
| A9 | ENSESTT00000028296 | | 6 | 39118691 | 39129820 |
| A9 | OTTHUMT00006006446 | dJ202I21.1-001 | 6 | 39118695 | 39124203 |
| A9 | OTTHUMT00006006448 | dJ202I21.1-003 | 6 | 39118695 | 39129820 |
| A9 | ENST00000229903 | C6orf64 | 6 | 39119524 | 39129720 |
| A9 | OTTHUMT00006006454 | dJ202I21.5-001 | 6 | 39124952 | 39127583 |
| A9 | OTTHUMT00006006447 | dJ202I21.1-002 | 6 | 39127706 | 39129702 |
| A9 | ENSESTT00000028297 | | 6 | 39127885 | 39129702 |
| A9 | OTTHUMT00006006456 | KCNK5-001 | 6 | 39203604 | 39244081 |
| A9 | ENST00000297169 | KCNK5 | 6 | 39205521 | 39243742 |
| A9 | ENSESTT00000028294 | | 6 | 39205943 | 39243759 |
| A9 | ENSESTT00000028295 | | 6 | 39208772 | 39243759 |
| A9 | OTTHUMT00006006464 | KCNK17-001 | 6 | 39313632 | 39329053 |
| A9 | ENSESTT00000028293 | | 6 | 39314030 | 39315137 |
| A9 | ENSESTT00000028292 | | 6 | 39314058 | 39319209 |
| A9 | ENST00000244759 | KCNK17 | 6 | 39314058 | 39328951 |
| A9 | OTTHUMT00006006462 | KCNK16-001 | 6 | 39329336 | 39337171 |
| A9 | ENSESTT00000028290 | | 6 | 39329579 | 39337599 |
| A9 | ENST00000211196 | KCNK16 | 6 | 39329633 | 39337171 |
| A9 | ENSESTT00000028291 | | 6 | 39332507 | 39337185 |
| A9 | ENSESTT00000028289 | | 6 | 39350785 | 39358362 |
| A9 | OTTHUMT00006006460 | dJ137F1.4-001 | 6 | 39350785 | 39400304 |
| A9 | ENST00000297170 | C6orf102 | 6 | 39358338 | 39445790 |
| A9 | ENST00000229913 | Q86T87 | 6 | 39358338 | 39554716 |
| A9 | OTTHUMT00006006458 | dJ137F1.3-001 | 6 | 39368378 | 39368803 |
| A9 | OTTHUMT00006006466 | dJ188D3.1-001 | 6 | 39434570 | 39445946 |
| A9 | OTTHUMT00006006470 | dJ1043E3.1-001 | 6 | 39554634 | 39740037 |
| A9 | ENSESTT00000028286 | | 6 | 39554831 | 39599605 |
| A9 | ENSESTT00000028284 | | 6 | 39554831 | 39601027 |
| A9 | OTTHUMT00006006472 | dJ1043E3.1-003 | 6 | 39559134 | 39592907 |
| A9 | ENST00000287152 | | 6 | 39559160 | 39739941 |
| A9 | ENSESTT00000028288 | | 6 | 39559179 | 39592907 |
| A9 | ENSESTT00000028285 | | 6 | 39559179 | 39600971 |
| A9 | OTTHUMT00006006468 | dJ1043E3.2-001 | 6 | 39568448 | 39569574 |
| A9 | ENSESTT00000028287 | | 6 | 39597804 | 39599586 |
| A9 | OTTHUMT00006006471 | dJ1043E3.1-002 | 6 | 39597806 | 39610891 |
| A9 | ENSESTT00000028283 | | 6 | 39610746 | 39689117 |
| A9 | OTTHUMT00006006473 | dJ1043E3.1-004 | 6 | 39705830 | 39729590 |
| A9 | ENSESTT00000028282 | | 6 | 39706266 | 39739928 |
| A9 | ENSESTT00000028269 | | 6 | 39807005 | 39879702 |
| A9 | ENST00000274867 | DAAM2 | 6 | 39807649 | 39919503 |
| A9 | ENSESTT00000028270 | | 6 | 39836736 | 39879669 |
| A9 | ENSESTT00000028271 | | 6 | 39863403 | 39875800 |
| A9 | OTTHUMT00006006489 | dJ278E11.1-006 | 6 | 39870878 | 39876106 |
| A9 | OTTHUMT00006006485 | dJ278E11.1-002 | 6 | 39870878 | 39882152 |
| A9 | OTTHUMT00006006484 | dJ278E11.1-001 | 6 | 39870878 | 39919499 |
| A9 | OTTHUMT00006006486 | dJ278E11.1-003 | 6 | 39882351 | 39919496 |
| A9 | ENSESTT00000028272 | | 6 | 39884968 | 39893094 |
| A9 | OTTHUMT00006006487 | dJ278E11.1-004 | 6 | 39887977 | 39893404 |
| A9 | OTTHUMT00006006488 | dJ278E11.1-005 | 6 | 39902036 | 39905992 |
| A9 | OTTHUMT00006006480 | bA61I13.3-001 | 6 | 39903421 | 39911984 |
| A9 | OTTHUMT00006006481 | bA61I13.3-002 | 6 | 39903505 | 39912011 |
| A9 | ENSESTT00000028281 | | 6 | 39903505 | 39912011 |
| A9 | ENSESTT00000028273 | | 6 | 39911507 | 39913571 |
| A9 | ENSESTT00000028274 | | 6 | 39911802 | 39919761 |
| A9 | OTTHUMT00006006478 | bA61I13.2-001 | 6 | 39911971 | 39914702 |
| A9 | ENSESTT00000028279 | | 6 | 39917315 | 39919496 |
| A9 | ENSESTT00000028280 | | 6 | 39917519 | 39918030 |
| A9 | OTTHUMT00006006504 | MOCS1-005 | 6 | 39919690 | 39926973 |
| A9 | OTTHUMT00006006505 | MOCS1-006 | 6 | 39919690 | 39927603 |
| A9 | OTTHUMT00006006506 | MOCS1-007 | 6 | 39919690 | 39927603 |
| A9 | OTTHUMT00006006502 | MOCS1-003 | 6 | 39919690 | 39941994 |
| A9 | OTTHUMT00006006500 | MOCS1-001 | 6 | 39919690 | 39942310 |
| A9 | OTTHUMT00006006501 | MOCS1-002 | 6 | 39919690 | 39949091 |
| A9 | OTTHUMT00006006507 | MOCS1-008 | 6 | 39919690 | 39949106 |
| A9 | ENSESTT00000028275 | | 6 | 39920144 | 39926919 |
| A9 | ENST00000274884 | MOCS1 | 6 | 39920154 | 39949011 |
| A9 | ENST00000308559 | MOCS1 | 6 | 39920988 | 39942172 |
| A9 | ENSESTT00000028278 | | 6 | 39921368 | 39928029 |
| A9 | ENSESTT00000028276 | | 6 | 39924433 | 39949106 |
| A9 | OTTHUMT00006006503 | MOCS1-004 | 6 | 39926863 | 39947026 |
| A9 | ENSESTT00000028277 | | 6 | 39941918 | 39949056 |
| A9 | ENST00000320371 | | 6 | 39973005 | 39973427 |
| A9 | OTTHUMT00006006496 | dJ278E11.3-001 | 6 | 39973008 | 39973427 |
| A9 | OTTHUMT00006006498 | dJ278E11.5-001 | 6 | 40007409 | 40014827 |
| A9 | ENST00000333628 | | 6 | 40014193 | 40014863 |
| A9 | OTTHUMT00006006516 | bA552E20.1-001 | 6 | 40286160 | 40290831 |
| A9 | OTTHUMT00006006524 | bA535K1.1-003 | 6 | 40349548 | 40394479 |
| A9 | OTTHUMT00006006525 | bA535K1.1-004 | 6 | 40355059 | 40394479 |
| A9 | ENSESTT00000026515 | | 6 | 40358936 | 40360627 |
| A9 | OTTHUMT00006006518 | bA552E20.3-001 | 6 | 40358938 | 40360745 |
| A9 | OTTHUMT00006006520 | bA552E20.4-001 | 6 | 40373338 | 40384068 |
| A9 | OTTHUMT00006006522 | bA535K1.1-001 | 6 | 40392931 | 40394479 |
| A9 | OTTHUMT00006006523 | bA535K1.1-002 | 6 | 40392931 | 40394479 |
| A9 | ENSESTT00000026510 | | 6 | 40392944 | 40394492 |
| A9 | ENSESTT00000026509 | | 6 | 40393305 | 40394492 |
| A9 | OTTHUMT00006006532 | bA535K1.2-001 | 6 | 40406180 | 40602059 |
| A9 | ENST00000287126 | Q9ULH4 | 6 | 40406228 | 40601981 |
| A9 | ENSESTT00000026512 | | 6 | 40447652 | 40601916 |
| A9 | OTTHUMT00006006530 | bA121P10.1-001 | 6 | 40516225 | 40516987 |
| A9 | ENSESTT00000026514 | | 6 | 40516225 | 40516987 |
| A9 | OTTHUMT00006006534 | dJ462C17.1-001 | 6 | 40530930 | 40538527 |
| A9 | ENSESTT00000026513 | | 6 | 40530930 | 40538527 |
| A9 | OTTHUMT00006006536 | bA570K4.1-001 | 6 | 40728005 | 40729957 |
| A9 | ENSESTT00000026511 | | 6 | 40728005 | 40729957 |
| A10 | ENSESTT00000052482 | | 18 | 71312402 | 71313353 |
| A10 | ENSESTT00000052481 | | 18 | 71536395 | 71551301 |
| A10 | ENSESTT00000052480 | | 18 | 71962357 | 71963077 |
| A10 | ENSESTT00000056790 | | 18 | 72048442 | 72054756 |
| A10 | ENSESTT00000056791 | | 18 | 72098098 | 72099051 |
| A10 | ENST00000217537 | Y222_HUMAN | 18 | 72199207 | 72334123 |
| A10 | ENSESTT00000056807 | | 18 | 72201199 | 72217929 |
| A10 | ENSESTT00000056806 | | 18 | 72201199 | 72218100 |
| A10 | ENSESTT00000056792 | | 18 | 72276438 | 72279133 |
| A10 | ENSESTT00000056793 | | 18 | 72326067 | 72326639 |
| A10 | ENSESTT00000056794 | | 18 | 72367851 | 72401147 |
| A10 | ENSESTT00000056795 | | 18 | 72688458 | 72707773 |
| A10 | ENST00000253159 | ZNF236 | 18 | 72688458 | 72774998 |
| A10 | ENST00000320610 | ZNF236 | 18 | 72688458 | 72807272 |
| A10 | ENSESTT00000056797 | | 18 | 72717002 | 72734201 |
| A10 | ENSESTT00000056796 | | 18 | 72717002 | 72734313 |
| A10 | ENSESTT00000056798 | | 18 | 72737983 | 72747435 |
| A10 | ENSESTT00000056799 | | 18 | 72815875 | 72819321 |
| A10 | ENST00000318747 | MBP | 18 | 72817767 | 72855988 |
| A10 | ENSESTT00000056804 | | 18 | 72819098 | 72855989 |
| A10 | ENSESTT00000056803 | | 18 | 72819230 | 72856028 |
| A10 | ENSESTT00000056805 | | 18 | 72819273 | 72855988 |
| A10 | ENST00000281193 | | 18 | 72819360 | 72828993 |
| A10 | ENSESTT00000056800 | | 18 | 72820171 | 72823085 |
| A10 | ENSESTT00000056801 | | 18 | 72820171 | 72823085 |
| A10 | ENSESTT00000056802 | | 18 | 72855561 | 72905291 |
| A10 | ENST00000309607 | | 18 | 72867119 | 72867493 |
| A10 | ENST00000299727 | GALR1 | 18 | 73088710 | 73109070 |
| A10 | ENSESTT00000052501 | | 18 | 73089280 | 73095131 |
| A10 | ENSESTT00000052502 | | 18 | 73092673 | 73095150 |
| A10 | ENSESTT00000052503 | | 18 | 73461485 | 73463448 |
| A10 | ENSESTT00000052478 | | 18 | 74655974 | 74657295 |
| A10 | ENSESTT00000052477 | | 18 | 74836231 | 74837820 |
| A10 | ENST00000299466 | SALL3 | 18 | 74839252 | 74857165 |
| A10 | ENSESTT00000052474 | | 18 | 74853768 | 74856489 |
| A10 | ENSESTT00000052475 | | 18 | 74854402 | 74861654 |
| A10 | ENST00000307671 | ATP9B | 18 | 74928369 | 75236360 |
| A10 | ENSESTT00000052476 | | 18 | 74928431 | 74972951 |
| A10 | ENSESTT00000065967 | | 18 | 75162658 | 75191977 |
| A10 | ENSESTT00000065966 | | 18 | 75162658 | 75191981 |
| A10 | ENSESTT00000065968 | | 18 | 75195603 | 75237133 |
| A10 | ENSESTT00000065969 | | 18 | 75203116 | 75204817 |
| A10 | ENSESTT00000065970 | | 18 | 75203163 | 75237133 |
| A10 | ENST00000253506 | NFATC1 | 18 | 75254749 | 75345913 |
| A10 | ENSESTT00000065971 | | 18 | 75255250 | 75270171 |
| A10 | ENST00000329101 | NM_172387 | 18 | 75259369 | 75345913 |
| A10 | ENSESTT00000065975 | | 18 | 75270434 | 75311108 |
| A10 | ENSESTT00000065973 | | 18 | 75270434 | 75327154 |
| A10 | ENSESTT00000065974 | | 18 | 75270434 | 75327154 |
| A10 | ENSESTT00000065972 | | 18 | 75270434 | 75345394 |
| A10 | ENSESTT00000065976 | | 18 | 75306857 | 75310062 |
| A10 | ENSESTT00000065977 | | 18 | 75307549 | 75310062 |
| A10 | ENSESTT00000065978 | | 18 | 75310647 | 75327154 |
| A10 | ENST00000334423 | | 18 | 75314345 | 75314808 |
| A10 | ENST00000334083 | | 18 | 75363755 | 75810501 |
| A10 | ENST00000333925 | | 18 | 75363758 | 75550986 |
| A10 | ENSESTT00000065979 | | 18 | 75386139 | 75387291 |
| A10 | ENSESTT00000066012 | | 18 | 75386888 | 75388300 |
| A10 | ENSESTT00000066011 | | 18 | 75403966 | 75404501 |
| A10 | ENST00000327986 | | 18 | 75415199 | 75415865 |
| A10 | ENST00000317008 | NM_182570 | 18 | 75500963 | 75538593 |
| A10 | ENST00000299543 | CTDP1 | 18 | 75538778 | 75613484 |
| A10 | ENST00000075430 | NM_048368 | 18 | 75538925 | 75595497 |
| A10 | ENSESTT00000066010 | | 18 | 75561698 | 75562628 |
| A10 | ENSESTT00000065980 | | 18 | 75563836 | 75573932 |
| A10 | ENSESTT00000065981 | | 18 | 75573941 | 75613484 |
| A10 | ENSESTT00000065982 | | 18 | 75588015 | 75613484 |
| A10 | ENSESTT00000065983 | | 18 | 75594535 | 75613484 |
| A10 | ENST00000316249 | KCNG2 | 18 | 75722645 | 75758793 |
| A10 | ENSESTT00000066009 | | 18 | 75761442 | 75762272 |
| A10 | ENSESTT00000066008 | | 18 | 75761446 | 75762510 |
| A10 | ENST00000316111 | NM_025078 | 18 | 75761465 | 75810007 |
| A10 | ENSESTT00000066004 | | 18 | 75762569 | 75809829 |
| A10 | ENSESTT00000066005 | | 18 | 75762569 | 75809829 |
| A10 | ENSESTT00000066002 | | 18 | 75762569 | 75809842 |
| A10 | ENSESTT00000065999 | | 18 | 75762569 | 75809843 |
| A10 | ENSESTT00000066000 | | 18 | 75762569 | 75809843 |
| A10 | ENSESTT00000066006 | | 18 | 75762650 | 75809829 |
| A10 | ENSESTT00000066001 | | 18 | 75762650 | 75809843 |
| A10 | ENST00000262199 | Q9H6D0 | 18 | 75762953 | 75802393 |
| A10 | ENSESTT00000066003 | | 18 | 75763093 | 75809836 |
| A10 | ENSESTT00000066007 | | 18 | 75774508 | 75775293 |
| A10 | ENSESTT00000065984 | | 18 | 75823637 | 75826139 |
| A10 | ENSESTT00000065998 | | 18 | 75825557 | 75831889 |
| A10 | ENST00000269601 | DIM1_HUMAN | 18 | 75831846 | 75847511 |
| A10 | ENSESTT00000065996 | | 18 | 75832383 | 75836894 |
| A10 | ENSESTT00000065991 | | 18 | 75832383 | 75847550 |
| A10 | ENSESTT00000065993 | | 18 | 75832384 | 75847452 |
| A10 | ENSESTT00000065995 | | 18 | 75832396 | 75847330 |
| A10 | ENSESTT00000065994 | | 18 | 75832490 | 75847391 |
| A10 | ENSESTT00000065997 | | 18 | 75832541 | 75835766 |
| A10 | ENSESTT00000065992 | | 18 | 75832715 | 75847514 |
| A10 | ENST00000306735 | NM_024805 | 18 | 75893335 | 75905364 |
| A10 | ENSESTT00000065986 | | 18 | 75893389 | 75901198 |
| A10 | ENSESTT00000065985 | | 18 | 75893389 | 75905374 |
| A10 | ENSESTT00000065990 | | 18 | 75893434 | 75905357 |
| A10 | ENST00000262197 | Q8WZ65 | 18 | 75893473 | 75904914 |
| A10 | ENSESTT00000065987 | | 18 | 75896424 | 75905374 |
| A10 | ENSESTT00000065988 | | 18 | 75926233 | 75938117 |
| A10 | ENST00000262198 | NM_014913 | 18 | 75966162 | 75997201 |
| A10 | ENSESTT00000065989 | | 18 | 75989961 | 75992868 |
| A10 | ENSESTT00000052575 | | 18 | 76004909 | 76006133 |
| A10 | ENST00000314741 | PARD6G | 18 | 76016631 | 76104208 |
| A10 | ENSESTT00000052576 | | 18 | 76016997 | 76104406 |
| A10 | ENSESTT00000052577 | | 18 | 76039710 | 76059638 |
| A11 | ENST00000319217.1 | MPDZ | 9 | 13096497 | 13240357 |
| A11 | ENST00000319198.3 | MPDZ | 9 | 13096497 | 13240357 |
| A11 | ENST00000276902.1 | NFIB | 9 | 14077605 | 14297550 |
| A11 | ENST00000331870.1 | | 9 | 14194247 | 14194570 |
| A11 | ENST00000324876.2 | NM_178566 | 9 | 14607036 | 14683474 |
| A11 | ENST00000276911.1 | CER1 | 9 | 14710088 | 14712670 |
| A11 | ENST00000297595.3 | NM_144966 | 9 | 14727207 | 14900993 |
| A11 | ENST00000324457.3 | NM_144966 | 9 | 14727207 | 14900993 |
| A11 | ENST00000297593.1 | NM_144966 | 9 | 14727207 | 14900993 |
| A11 | ENST00000297615.2 | NM_152574 | 9 | 15161561 | 15297250 |
| A11 | ENST00000336042.1 | NM_152574 | 9 | 15161561 | 15297250 |
| A11 | ENST00000297627.1 | SNAPC3 | 9 | 15412732 | 15455830 |
| A11 | ENST00000336277.1 | SNAPC3 | 9 | 15412732 | 15455830 |
| A11 | ENST00000297635.1 | PSIP2 | 9 | 15454067 | 15500982 |
| A11 | ENST00000285482.4 | | 9 | 15542895 | 15613411 |
| A11 | ENST00000297641.1 | NM_173550 | 9 | 15734586 | 15961895 |
| A11 | ENST00000318677.2 | NM_173550 | 9 | 15734586 | 15961895 |
| A11 | ENST00000309604.2 | NM_017637 | 9 | 16408579 | 16427061 |
| A11 | ENST00000317612.2 | NM_017637 | 9 | 16408579 | 16427061 |
| A11 | ENST00000316584.1 | NM_152576 | 9 | 16517183 | 16517287 |
| A11 | ENST00000297642.1 | NM_017738 | 9 | 17125064 | 17485003 |
| A11 | ENST00000262360.2 | NM_017738 | 9 | 17125064 | 17485003 |

What is claimed is:

1. A method for identifying an antineoplastic agent, comprising:
(a) contacting a test compound with a cell that expresses one or more amplicons of Table 2 having an amplification ratio of at least 2.0; and
(b) determining a change in said amplification ratio due to said contacting;
wherein a change in said amplification ratio due to said contacting indicates that said test compound has gene modulating activity
thereby identifying said test compound as a gene modulating agent.

2. The method of claim 1 wherein said change in expression is a decrease in expression.

3. The method of claim 2 wherein said decrease in expression is a decrease in copy number of the gene.

4. The method of claim 1 wherein said cell was genetically engineered to express said amplicon.

5. A method for identifying an antineoplastic agent, comprising:
(a) contacting a test compound with a cell that expresses at least one gene corresponding to a polynucleotide comprising a nucleotide sequence of SEQ ID NO: 1-3049 and under conditions promoting expression of said gene; and
(b) determining a change in expression of said gene as a result of said contacting
wherein a change in expression indicates gene modulation thereby identifying said test compound as a gene modulating agent.

6. The method of claim 5 wherein said change in expression is a decrease in expression.

7. The method of claim 5 wherein said decrease in expression is a decrease in copy number of the gene.

8. The method of claim 5 wherein said gene comprises a nucleotide sequence of one of SEQ ID NO: 1-3049.

9. The method of claim 5 wherein said cell was genetically engineered to express said gene.

10. A method for detecting the cancerous status of a cell, comprising detecting elevated expression in said cell of at least one gene corresponding to a polynucleotide comprising a nucleotide sequence of SEQ ID NO: 1-3049 whereby such elevated expression is indicative of cancerous status of the cell.

11. The method of claim 10 wherein said elevated expression is an elevated copy number of the gene.

12. A method for identifying a compound as an anti-neoplastic agent, comprising:
(a) contacting a test compound with a polypeptide encoded by a gene selected from SEQ ID NO: 1-3049,
(b) determining a change in a biological activity of said polypeptide due to said contacting,
wherein a change in activity indicates anti-neoplastic activity and thereby identifies such test compound as an agent having antineoplastic activity.

13. The method of claim 12 wherein said change in biological activity is a decrease in biological activity.

14. The method of claim 12 wherein said biological activity is an enzyme activity.

15. The method of claim 14 wherein said enzyme is selected from kinase, protease, peptidase, phosphodiesterase, phosphatase, dehydrogenase, reductase, carboxylase, transferase, deacetylase and polymerase.

16. The method of claim 15 wherein said kinase is a protein kinase.

17. The method of claim 15 wherein said kinase is a serine or threonine kinase.

18. The method of claim 15 wherein said kinase is a receptor tyrosine protein kinase.

19. The method of claim 15 wherein said protease is a serine protease, cysteine protease or aspartic acid protease.

20. The method of claim 15 wherein said transferase is a methyltransferase.

21. The method of claim 20 wherein said methyl transferase is a cytidine methyltransferase or an adenine methyltransferase.

22. The method of claim 15 wherein said deacetylase is a histone deacetylase.

23. The method of claim 11 wherein said carboxylase is a γ-carboxylase.

24. The method of claim 15 wherein said peptidase is a zinc peptidase.

25. The method of claim 15 wherein said polymerase is a DNA polymerase.

26. The method of claim 15 wherein said polymerase is a RNA polymerase.

27. The method of claim 12 wherein said biological activity is a membrane transport activity.

28. The method of claim 12 wherein said polypeptide is a cation channel protein, an anion channel protein, a gated-ion channel protein or an ABC transporter protein.

29. The method of claim 12 wherein said polypeptide is an integrin.

30. The method of claim 12 wherein said polypeptide is a Cytochrome P450 enzyme.

31. The method of claim 12 wherein said polypeptide is a nuclear hormone receptor.

32. The method of claim 12 wherein said biological activity is a receptor activity.

33. The method of claim 12 wherein said receptor is a G-protein-coupled receptor.

34. The method of claim 12 wherein said polypeptide is contained in a cell.

35. A method for identifying an anti-neoplastic agent comprising contacting a cancerous cell with a compound found to have anti-neoplastic activity in the method of claim 12 under conditions promoting the growth of said cell and detecting a change in the activity of said cancerous cell.

36. The method of claim 35 wherein said change in activity is a decrease in the rate of replication of said cancerous cell.

37. The method of claim 35 wherein said change in activity is a decrease in the total number of progeny cells that can be produced by said cancerous cell.

38. The method of claim 35 wherein said change in activity is a decrease in the number of times said cancerous cell can replicate.

39. The method of claim 35 wherein said change in activity is the death of said cancerous cell.

40. A method for treating cancer comprising contacting a cancerous cell with an agent first identified as having gene modulating activity using the method of claim 1, 5, or 12 and in an amount effective to cause a reduction in cancerous activity of said cell.

41. The method of claim 40 wherein said cancerous cell is contacted in vivo.

42. The method of claim 40 wherein said reduction in cancerous activity is a decrease in the rate of proliferation of said cancerous cell.

43. The method of claim 40 wherein said reduction in cancerous activity is the death of said cancerous cell.

44. The method of claim 40 wherein said cancer is a cancer of breast, colon, lung or prostate tissues.

45. A method for treating cancer comprising contacting a cancerous cell with an agent having affinity for an expression product of a gene corresponding to a polynucleotide comprising a nucleotide sequence of SEQ ID NO: 1-3049 and in an amount effective to cause a reduction in cancerous activity of said cell.

46. The method of claim 45 wherein said expression product is a polypeptide.

47. The method of claim 45 wherein said agent is an antibody.

48. A method for monitoring the progress of cancer therapy in a patient comprising monitoring in a patient undergoing cancer therapy the expression of a gene corresponding to a polypeptide having a sequence selected from SEQ ID NO: 1-3049.

49. The method of claim 48 wherein said gene comprises a sequence of SEQ ID NO: 1-3049.

50. The method of claim 48 wherein said cancer therapy is chemotherapy.

51. The method of claim 48 wherein said cancer is a cancer of breast, colon, lung or prostate tissues.

52. A method for determining the likelihood of success of cancer therapy in a patient, comprising monitoring in a patient undergoing cancer therapy the expression of a gene corresponding to a polynucleotide having a sequence of one of SEQ ID NO: 1-3049 wherein a decrease in said expression prior to completion of said cancer therapy is indicative of a likelihood of success of said cancer therapy.

53. The method of claim 52 wherein said gene comprises a sequence of SEQ ID NO: 1-3049.

54. The method of claim 52 wherein said cancer therapy is chemotherapy.

55. The method of claim 52 wherein said cancer is a cancer of breast, colon, lung or prostate tissues.

56. A method for producing test data with respect to the anti-neoplastic activity of a compound comprising:
(a) identifying a test compound as having anti-neoplastic activity using a method of claim 12;
(b) producing test data with respect to the anti-neoplastic activity of said test compound sufficient to identify the chemical structure of said test compound.

57. A method for determining the progress of a treatment for cancer in a patient afflicted therewith, following commencement of a cancer treatment on said patient, comprising:
(a) determining in said patient a change in expression of one or more genes corresponding to a polynucleotide comprising a nucleotide sequence of SEQ ID NO: 1-3049; and
(b) determining a change in expression of said gene compared to expression of said one or more determined genes prior to commencement of said cancer treatment;
wherein said change in expression indicates progress of said treatment
thereby determining the progress of said treatment.

58. The method of claim 57 wherein said change in expression is a decrease in expression and said decrease indicates success of said treatment.

59. A method for determining the progress of a treatment for cancer in a patient afflicted therewith, following commencement of a cancer treatment on said patient, comprising:
(a) determining in said patient a change in expression of one or more genes corresponding to a polynucleotide comprising a nucleotide sequence of SEQ ID NO: 1-3049; and
(b) determining a change in expression of said gene compared to expression of said one or more determined genes prior to commencement of said cancer treatment;
wherein said change in expression indicates progress of said treatment
thereby determining the progress of said treatment.

60. The method of claim 59 wherein said change in expression is a decrease in expression and said decrease indicates success of said treatment.
